SEQUENCE LISTING (191 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, synthetic construct
RNAMF

SEQ ID NO: 67 - protein, 16 aa, synthetic construct
GIGTGGATSYADSVKG

SEQ ID NO: 68 - protein, 7 aa, synthetic construct
GRYYFDY

SEQ ID NO: 69 - protein, 12 aa, synthetic construct
RASQSVSSSYLA

SEQ ID NO: 70 - protein, 7 aa, synthetic construct
GASSRAT

SEQ ID NO: 71 - protein, 10 aa, synthetic construct
QQYDHSAGWT

SEQ ID NO: 72 - protein, 7 aa, synthetic construct
GASSRAP

SEQ ID NO: 73 - protein, 10 aa, synthetic construct
QQYGSSPPWT

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 77 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 78 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 80 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 81 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 82 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 83 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 84 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 90 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 91 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 92 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 95 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 96 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 97 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 98 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 99 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 100 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 101 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 103 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 104 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 105 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 106 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 108 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 109 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 110 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 111 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 112 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 113 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 114 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 115 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 116 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 117 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 118 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 119 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 120 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 121 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 122 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 123 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 124 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 125 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 126 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 127 - protein, 5 aa, synthetic construct
SYYMH

SEQ ID NO: 128 - protein, 17 aa, synthetic construct
IINPRGGSTSYAQKFQG

SEQ ID NO: 129 - protein, 7 aa, synthetic construct
GKYWMYD

SEQ ID NO: 130 - protein, 13 aa, synthetic construct
SGGGSSIGNSYVS

SEQ ID NO: 131 - protein, 7 aa, synthetic construct
DNNKRPS

SEQ ID NO: 132 - protein, 12 aa, synthetic construct
GTWDTSPVWEWP

SEQ ID NO: 133 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 134 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 135 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 136 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 137 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 138 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 139 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 140 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 141 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 142 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 143 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 144 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 145 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 146 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 147 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 148 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 149 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 150 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 151 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 152 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 153 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 154 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 155 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 156 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 157 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 158 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 159 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 160 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 164 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 165 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 166 - protein, 5 aa, synthetic construct
RHAMA

SEQ ID NO: 167 - protein, 17 aa, synthetic construct
AITSSGRSIYYADSVKG

SEQ ID NO: 168 - protein, 7 aa, synthetic construct
VHRAFDY

SEQ ID NO: 169 - protein, 13 aa, synthetic construct
SGSPLFPDSGSFN

SEQ ID NO: 170 - protein, 7 aa, synthetic construct
ADSHRPS

SEQ ID NO: 171 - protein, 11 aa, synthetic construct
GTWDYSLSGYV

SEQ ID NO: 172 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 173 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 174 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 175 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 176 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 177 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 178 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 179 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 180 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 181 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 182 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 183 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 184 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 185 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 186 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 187 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 188 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 189 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 190 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 191 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

(19) United States
(12) Patent Application Publication
(10) Pub. No.: US 2023/0146317 A1
(43) Pub. Date: May 11, 2023

(54) METHODS FOR TREATING CHRONIC SPONTANEOUS URTICARIA BY ADMINISTERING AN IL-4R ANTAGONIST

(71) Applicants: SANOFI BIOTECHNOLOGY, Paris (FR); REGENERON PHARMACEUTICALS, INC., Tarrytown, NY (US)

(72) Inventors: Nikhil Amin, Tarrytown, NY (US); Elizabeth Laws, Paris (FR); Leda Mannent, Paris (FR); Allen Radin, Tarrytown, NY (US); Aleksandra Stjepanovic, Paris (FR); Naimish Patel, Bridgewater, NJ (US); Heribert Staudinger, Bridgewater, NJ (US)

(21) Appl. No.: 17/872,225

(22) Filed: Jul. 25, 2022

Related U.S. Application Data
(60) Provisional application No. 63/353,654, filed on Jun. 20, 2022.
(60) Provisional application No. 63/313,041, filed on Feb. 23, 2022.
(60) Provisional application No. 63/240,734, filed on Sep. 3, 2021.
(60) Provisional application No. 63/225,716, filed on Jul. 26, 2021.

(30) Foreign Application Priority Data
Mar. 4, 2022 (EP) 22315049.1

(51) Int. Cl. C07K 16/24; A61K 31/4174; A61K 9/00; A61P 17/00

(52) U.S. Cl. CPC C07K 16/247; A61K 9/0019; A61K 9/0053; A61K 31/4174; A61P 17/00; A61K 2039/545

(57) ABSTRACT

Methods for treating or preventing chronic spontaneous urticaria in a subject are provided. Methods comprising administering to a subject in need thereof a therapeutic composition comprising an interleukin-4 receptor (IL-4R) antagonist, such as an anti-IL-4R antibody or antigen-binding fragment thereof, are provided.

### RELATED APPLICATIONS

This application claims the benefit of U.S. Provisional Application No. 63/225,716, filed Jul. 26, 2021, U.S. Provisional Application No. 63/240,734, filed Sep. 3, 2021, U.S. Provisional Application No. 63/313,041, filed Feb. 23, 2022, U.S. Provisional Application No. 63/353,654, filed Jun. 20, 2022, and EP Priority Application No. 22315049.1, filed Mar. 4, 2022, the contents of which are incorporated by reference in their entireties for all purposes.

### SEQUENCE LISTING

[0001.1] The instant application contains a Sequence Listing which has been submitted electronically in XML file format and is hereby incorporated by reference in its entirety. Said XML copy, created on Oct. 6, 2022, is named 731156_SA9-281_SL.xml and is 183,747 bytes in size.

### FIELD OF THE INVENTION

The disclosure relates to the treatment and/or prevention of chronic spontaneous urticaria (CSU) in a subject in need thereof. The disclosure relates to the administration of an interleukin-4 receptor (IL-4R) antagonist to treat or prevent CSU in a subject in need thereof.

### BACKGROUND

Chronic spontaneous urticaria, formerly also known as chronic idiopathic urticaria and chronic urticaria, is one of the most frequent skin diseases. At any time, 0.5% to 1% of the population suffers from the disease. (See Maurer M, et al. Unmet clinical needs in chronic spontaneous urticaria. A GA(2)LEN task force report. Allergy. 2011;66(3):317-30.) It is characterized by the spontaneous appearance of pruritic wheals (hives) and flare-type skin reactions persisting for more than 6 weeks without a specific known cause, which may be accompanied by angioedema. Although all age groups can be affected, the peak incidence is seen between 20 and 40 years of age. The duration of the disease is generally several years but is likely to be longer in more severe cases, cases with concurrent angioedema, in combination with physical urticaria, or with a positive autologous serum skin test (auto-reactivity). Chronic spontaneous urticaria has major detrimental effects on quality-of-life, with sleep deprivation and psychiatric comorbidity being frequent. It also has a large impact on society in terms of direct and indirect health care costs as well as reduced performance at work and in private life. (See Id.)

Chronic spontaneous urticaria patients with and without angioedema experience debilitating hives and pruritus secondary to mast cell and basophil dysregulation. Degranulation of these cell types by Fc gamma receptor (FcεRI) activation, through agonistic autoantibodies or cell surface-bound immunoglobulin E (IgE) cross-linked by antigen, release histamine and other pro-inflammatory mediators leading to local tissue edema and pruritus. Many symptoms of urticaria are mediated primarily by the actions of histamine (a mast cell mediator) on the H1-receptors, and treatment with H1-antihistamines (H1-AH) is a mainstay of therapy. (See Zuberbier T, et al. The EAACI/GA2LEN/EDF/WAO guideline for the definition, classification, diagnosis and management of urticaria. Allergy. 2018;73(7):1393-414.) Approximately 50% of patients achieve symptomatic control with conventional H1-AH therapy. (See Kaplan AP. Chronic spontaneous urticaria: pathogenesis and treatment considerations. Allergy Asthma Immunol Res. 2017;9(6):477-82.) Even with up-titration of antihistamines, approximately 40% to 50% of patients remain symptomatic. The mechanism by which omalizumab exerts its therapeutic effects is likely constrained to reduction in serum IgE and consequent down-regulation of IgE receptors. Targeting IgE by omalizumab has been successful in treating CSU patients but not all patients are equally responsive to this therapy. (See Maurer M, et al. Omalizumab for the treatment of chronic idiopathic or spontaneous urticaria. N Engl J Med. 2013;368(10):924-35.) Therefore, there remains an unmet need. Accordingly, a need exists for novel therapies to treat CSU.

### BRIEF SUMMARY OF THE INVENTION

In one aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject was previously ineffectively treated with H1 antihistamine therapy and anti-IgE antibody therapy, is provided.

In certain exemplary embodiments, the subject remains symptomatic despite the use of H1 antihistamine.

In certain exemplary embodiments, an H1 antihistamine is administered in combination with the antibody or an antigen-binding fragment thereof. In certain exemplary embodiments, the H1 antihistamine is selected from the group consisting of cetirizine, levocetirizine, fexofenadine, loratadine, desloratadine, bilastine, and rupatadine.

In certain exemplary embodiments, the subject is intolerant to omalizumab or remains symptomatic despite the use of omalizumab.

In certain exemplary embodiments, the subject is an adult. In certain exemplary embodiments, the antibody or antigen-binding fragment thereof is administered to the subject as an initial dose followed by one or more secondary doses. In certain exemplary embodiments, the initial dose is about 600 mg and each secondary dose is about 300 mg. In certain exemplary embodiments, each secondary dose is administered every 2 weeks.

In certain exemplary embodiments, the subject is 12 years old to less than 18 years old. In certain exemplary embodiments, the antibody or antigen-binding fragment thereof is administered to the subject as an initial dose followed by one or more secondary doses. In certain exemplary embodiments, each secondary dose is administered every 2 weeks.

In certain exemplary embodiments, the subject has a body weight of at least 60 kg and the initial dose is about 600 mg and each secondary dose is about 300 mg. In certain exemplary embodiments, each secondary dose is administered every 2 weeks.

In certain exemplary embodiments, the subject is 6 years old to less than 12 years old. In certain exemplary embodiments, the antibody or antigen-binding fragment thereof is administered to the subject as an initial dose followed by one or more secondary doses.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject has a body weight of less than 30 kg and at least 15 kg and the initial dose is about 300 mg and each secondary dose is about 300 mg. In certain exemplary embodiments, each secondary dose is administered every 4 weeks. In certain exemplary embodiments, the subject is at least 2 years old and less than 6 years old.

In certain exemplary embodiments, the subject has a body weight of less than 15 kg and at least 5 kg and the initial dose is about 200 mg and each secondary dose is about 200 mg. In certain exemplary embodiments, each secondary dose is administered every 4 weeks. In certain exemplary embodiments, the subject is at least 2 years old and less than 6 years old. In certain exemplary embodiments, the subject is at least 2 years old and less than 12 years old. In certain exemplary embodiments, the subject is at least 6 years old and less than 12 years old.

In certain exemplary embodiments, the body weight of the subject is less than 60 kg. in certain exemplary embodiments, the subject has a body weight of between at least 30 kg and less than 60 kg.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is 12 years old to less than 18 years old, wherein the antibody or antigen-binding fragment thereof is administered to the subject as an initial dose followed by one or more secondary doses, and wherein the antibody or antigen-binding fragment thereof is administered to the subject in a weight-dependent dosage, is provided.

In certain exemplary embodiments, the subject has a body weight of less than 60 kg and wherein the initial dose is about 400 mg and each secondary dose is about 200 mg.

In certain exemplary embodiments, the subject has a body weight of at least 60 kg and wherein the initial dose is about 600 mg and each secondary dose is about 300 mg.

In certain exemplary embodiments, the subject has a body weight of less than 60 kg.

In certain exemplary embodiments, each secondary dose is administered every 2 weeks.

In certain exemplary embodiments, the subject has a body weight of between at least 5 kg and less than 15 kg, wherein the initial dose is about 200 mg and each secondary dose is about 200 mg, and wherein each secondary dose is administered every four weeks.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is 6 years old to less than 12 years old, wherein the antibody or antigen-binding fragment thereof is administered to the subject as an initial dose followed by one or more secondary doses, and wherein the antibody or antigen-binding fragment thereof is administered to the subject in a weight-dependent dosage, is provided.

In certain exemplary embodiments, the subject has a body weight of at least 30 kg and wherein the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and wherein the initial dose is about 600 mg and each secondary dose is about 300 mg. In certain exemplary embodiments, each secondary dose is administered every 4 weeks.

In certain exemplary embodiments, the subject has a body weight of less than 60 kg.

In certain exemplary embodiments, the subject has a body weight of between at least 5 kg and less than 15 kg, wherein the initial dose is about 200 mg and each secondary dose is about 200 mg, and wherein each secondary dose is administered every four weeks.

In certain exemplary embodiments, an H1 antihistamine is administered in combination with the antibody or an antigen-binding fragment thereof.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 12 years old to less than 18 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with antihistamine therapy, is provided.

In certain exemplary embodiments, the subject has a body weight of less than 60 kg and wherein the initial dose is about 400 mg and each secondary dose is about 200 mg.

In certain exemplary embodiments, the subject has a body weight of at least 60 kg and wherein the initial dose is about 600 mg and each secondary dose is about 300 mg.

In certain exemplary embodiments, the subject has a body weight of less than 60 kg.

In certain exemplary embodiments, each secondary dose is administered every 2 weeks.

In certain exemplary embodiments, the subject has a body weight of between at least 5 kg and less than 15 kg, wherein the initial dose is about 200 mg and each secondary dose is about 200 mg, and wherein each secondary dose is administered every four weeks.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 6 years old to less than 12 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with antihistamine therapy, is provided.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject has a body weight of less than 60 kg.

In certain exemplary embodiments, the subject has a body weight of between at least 5 kg and less than 15 kg, wherein the initial dose is about 200 mg and each secondary dose is about 200 mg, and wherein each secondary dose is administered every four weeks.

In certain exemplary embodiments, the subject remains symptomatic despite the use of H1 antihistamine.

In certain exemplary embodiments, an H1 antihistamine is administered in combination with the antibody or an antigen-binding fragment thereof. In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, wherein the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 12 years old to less than 18 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with anti-IgE antibody therapy, is provided.

In certain exemplary embodiments, the subject has a body weight of less than 60 kg and the initial dose is about 400 mg and each secondary dose is about 200 mg.

In certain exemplary embodiments, the subject has a body weight of at least 60 kg and the initial dose is about 600 mg and each secondary dose is about 300 mg.

In certain exemplary embodiments, each secondary dose is administered every 2 weeks.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 6 years old to less than 12 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with anti-IgE antibody therapy, is provided.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject is intolerant to omalizumab or remains symptomatic despite the use of omalizumab.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 12 years old to less than 18 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with H1 antihistamine therapy and anti-IgE antibody therapy, is provided.

In certain exemplary embodiments, the subject has a body weight of less than 60 kg and the initial dose is about 400 mg and each secondary dose is about 200 mg.

In certain exemplary embodiments, the subject has a body weight of at least 60 kg and the initial dose is about 600 mg and each secondary dose is about 300 mg.

In certain exemplary embodiments, each secondary dose is administered every 2 weeks.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is the subject is 6 years old to less than 12 years old, wherein the antibody or antigen-binding fragment thereof is administered to the subject as an initial dose followed by one or more secondary doses, wherein the antibody or antigen-binding fragment thereof is administered to the subject in a weight dependent dosage, and wherein the subject was previously ineffectively treated with H1 antihistamine therapy and anti-IgE antibody therapy, is provided.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject is intolerant to omalizumab or remains symptomatic despite the use of omalizumab.

In certain exemplary embodiments, the subject remains symptomatic despite the use of H1 antihistamine.

In certain exemplary embodiments, an H1 antihistamine is administered in combination with the antibody or an antigen-binding fragment thereof. In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

In another aspect, a method for treating a subject having chronic spontaneous urticaria (CSU) is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the subject is 2 years old to less than 6 years old, wherein the antibody or antigen-binding fragment thereof is administered to the subject as an initial dose followed by one or more secondary doses, and wherein the antibody or antigen-binding fragment thereof is administered to the subject in a weight-dependent dosage.

In certain exemplary embodiments, the subject has a body weight of between at least 15 kg and less than 30 kg, and the initial dose is about 300 mg and each secondary dose is about 300 mg. In certain exemplary embodiments, each secondary dose is administered every 4 weeks.

In certain exemplary embodiments, an H1 antihistamine is administered in combination with the antibody or an antigen-binding fragment thereof.

In certain exemplary embodiments, the subject has a body weight of between at least 5 kg and less than 15 kg, and the initial dose is about 200 mg and each secondary dose is about 200 mg. In certain exemplary embodiments, each secondary dose is administered every 4 weeks.

In certain exemplary embodiments, the subject has a body weight of between at least 30 kg and less than 60 kg, wherein the initial dose is about 400 mg and each secondary dose is about 200 mg, and wherein each secondary dose is administered every two weeks.

In certain exemplary embodiments, the subject remains symptomatic despite the use of H1 antihistamine.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

In certain exemplary embodiments, the PRO is itch severity score (ISS) and the subject has a decrease in itch severity score over 7 days (ISS7). In certain exemplary embodiments, the decrease in ISS7 is at least 5.

In certain exemplary embodiments, the PRO is hive severity score (HSS) and the subject has a decrease in hive severity score over 7 days (HSS7).

In certain exemplary embodiments, the PRO is urticaria activity score (UAS) and the subject has a decrease in urticaria activity score over 7 days (UAS7). In certain exemplary embodiments, the decrease in UAS7 is at least 10. In certain exemplary embodiments, the UAS7 of the subject is 0.

In certain exemplary embodiments, the PRO is urticaria activity score (UAS) and the UAS of the subject is 6 or less.

In certain exemplary embodiments, the PRO is angioedema activity score over 7 days (AAS7) and the subject has a decrease in AAS score.

In certain exemplary embodiments, the PRO is the urticaria control test (UCT) and the subject has an increase in UCT score. In certain exemplary embodiments, the UCT of the subject is 12 or greater.

In certain exemplary embodiments, the PRO is dermatology life quality index (DLQI) and the subject has a decrease in DLQI score.

In certain exemplary embodiments, the PRO is children’s dermatology life quality index (CDLQI) and the subject has a decrease in CDLQI score.

In certain exemplary embodiments, the PRO is chronic urticaria quality of life questionnaire (CU-Q2oL) and the subject has a decrease in CU-Q2oL score.

In certain exemplary embodiments, the PRO is patient global impression of change (PGIC) and the subject has a decrease in PGIC score.

In certain exemplary embodiments, the PRO is patient global impression of severity (PGIS) and the subject has a decrease in PGIS score.

In certain exemplary embodiments, the PRO is Euroqol-5 dimensions (EQ-5D) or Euroqol-5 dimensions Youth version (EQ-5D Y) and the subject has an increase in EQ visual analogue scale (EQ VAS) score.

In certain exemplary embodiments, the improvement in the PRO occurs with 12 weeks of treatment with the antibody or antigen-binding fragment thereof. In certain exemplary embodiments, the improvement in the PRO occurs with 24 weeks of treatment with the antibody or antigen-binding fragment thereof.

In certain exemplary embodiments, prior to treatment with the antibody or antigen-binding fragment thereof, the subject has an UAS7 score of 16 or more.

In certain exemplary embodiments, prior to treatment with the antibody or antigen-binding fragment thereof, the subject has an ISS7 score of 8 or more.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, wherein the treatment with the antibody or antigen-binding fragment thereof results in a decrease in the need for treatment of the subject with oral corticosteroids. In certain exemplary embodiments, the dosage of oral corticosteroids required is decreased. In certain exemplary embodiments, the number of days wherein oral corticosteroid treatment is required are decreased.

In certain exemplary embodiments, the treatment with the antibody or antigen-binding fragment thereof results in a decrease in the need for treatment of the subject with antihistamine rescue medication. In certain exemplary embodiments, the dosage of antihistamine rescue medication required is decreased. In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject does not have active atopic dermatitis or chronic inducible cold urticaria (CICU).

### BRIEF DESCRIPTION OF THE FIGURES

The foregoing and other features and advantages of the disclosure will be more fully understood from the following detailed description of illustrative embodiments taken in conjunction with the accompanying drawings.

FIG. 1 graphically depicts an overview of the study design of Example 1. Study A and C have participants who are omalizumab naive. Study B has participants who are intolerant or incomplete responders to omalizumab. Dupilumab 300 mg Q2W/Q4W, administered as 1 SC injection of dupilumab 300 mg (2 mL). Dupilumab 200 mg Q2W, administered as 1 SC injection of dupilumab 200 mg (1.14 mL). Matched placebo is prepared in the same formulation without the addition of protein (i.e., the active substance). Adults: 300 mg Q2W; Adolescents: 200 mg Q2W <60 kg or 300 mg ≥60 kg; (for Study A and Study C only) Children 6 to <12 years of age: 200 mg Q2W ≥60 kg or 300 mg Q4W <30 kg and ≥15 kg. EOS = end of study; EOT = end of treatment; R = randomization; SC = subcutaneous; Q2W = every 2 weeks; Q4W = every 4 weeks.

FIGS. 2A-C depict a table of the schedule of activities for the two randomized, placebo-controlled studies of dupilumab in patients with CSU who remain symptomatic despite the use of H1 antihistamine treatment (Example 1).

FIG. 3 depicts the questionnaire used for determining Urticaria Activity Score, a CSU-associated patient-recorded outcome measure.

FIG. 4 depicts the questionnaire used for the Urticaria Control Test, a CSU-associated patient-recorded outcome measure.

FIGS. 5A-5C depict the Chronic Urticaria Quality of Life Questionnaire, a CSU-associated patient-recorded outcome measure.

FIG. 6 depicts the overview of Study A. Study A includes participants who are omalizumab naive who are treated with dupilumab for 24 weeks.

FIG. 7 depicts the statistical testing hierarchy of Study A. The p values for the primary endpoints at 12 and 24 weeks are presented in this figure.

FIG. 8 graphically depicts a decrease in ISS7 in least square mean (LS mean) change from baseline at both weeks 12 and 24 for the dupilumab treatment group versus placebo.

FIG. 9 depicts a plot of mean change in ISS7 over time in both the placebo and dupilumab treatment groups from baseline to week 36.

FIG. 10 graphically depicts a decrease in UAS7 in least square mean (LS mean) change from baseline at both weeks 12 and 24 for the dupilumab treatment group versus placebo.

FIG. 11 depicts a plot of mean change in UAS7 over time in both the placebo and dupilumab treatment groups from baseline to week 36.

FIG. 12 graphically depicts the percentage of UAS7 partial responders (patients with UAS7 equal to or less than 6) in both the placebo and dupilumab treatment groups at 12 and 24 weeks. The dupilumab treatment group has a higher percentage of UAS7 partial responders at both time points.

FIG. 13 graphically depicts the percentage of UAS7 complete responders (patients with UAS7 equal to zero) in both the placebo and dupilumab treatment groups at 12 and 24 weeks. The dupilumab treatment group has a higher percentage of UAS7 complete responders at both time points.

FIG. 14 graphically depicts the percentage of patients that reached the ISS7 minimum important difference (MID) (patients with a decrease in ISS7 of 5 or greater) in both the placebo and dupilumab treatment groups at 12 and 24 weeks. The dupilumab treatment group has a higher percentage of patients that reached ISS7 MID at both time points.

FIG. 15 depicts a plot of the proportion of patients with an ISS7 reduction from baseline of 5 points or more over time in both the placebo and dupilumab treatment groups up to week 36.

### DETAILED DESCRIPTION

Before the disclosure is described, it is to be understood that disclosure is not limited to particular methods and experimental conditions described, as such methods and conditions may vary. It is also to be understood that the terminology used herein is for the purpose of describing particular embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the disclosures herein, the typical methods and materials are now described. All publications mentioned herein are incorporated herein by reference in their entirety.

The present disclosure provides methods and compositions for treating chronic spontaneous urticaria (CSU).

As used herein, “urticaria” refers to a skin condition characterized by the formation of wheal(s) (i.e., hive(s)) and/or the onset of angioedema that may last for a few minutes or many hours. As used herein “chronic urticaria” or “CU” refers to urticaria defined by recurrent episodes occurring at least twice a week for 6 weeks.

As used herein, “chronic spontaneous urticaria” or “CSU” refers to a subset of CU in which wheal(s) and/or angioedema are induced or provoked in a subject, over a period of at least six weeks, wherein the CSU has no specific cause or trigger.

As used herein, a “wheal” refers to a raised, itchy (i.e., pruritic) area of the skin. Wheal(s) may be used interchangeably with “hive(s).” Wheal intensity may be characterized using a variety of assessment tools known in the art, including those discussed below.

As used herein, “angioedema” refers to an area of swelling of the lower layer of skin and tissue just under the skin or mucous membranes. Swelling may occur, e.g., in the face, tongue, larynx, abdomen, arms and/or legs. Onset is typically over minutes to hours and typically resolves in hours to a few days.

### Methods for Improving CSU-Associated Patient-Recorded Outcome (PRO) Measures

Methods for improving one or more CSU-associated patient-recorded outcome (PRO) measures in a subject in need thereof, wherein the methods comprise administering a pharmaceutical composition comprising an IL-4R antagonist to the subject, are also provided.

Examples of CSU-associated PRO measures include: (1) urticaria activity score (UAS), (2) angioedema activity score (AAS), (3) urticaria control test (UCT) score, (4) dermatology life quality index (DLQI), (5) children’s dermatology quality life quality Index (CDLQI), (6) chronic urticaria quality of life questionnaire (CU-Q2oL) score, (7) patient global impression of change (PGIC), (8) patient global impression of severity (PGIS), (9) Euroqol-5 dimensions (EQ-5D) score, and (10) Euroqol-5 dimensions Youth version (EQ-5D Y) score.

An “improvement in a CSU-associated PRO measure” means an increase from baseline of one or more of UCT score and EQ visual analogue scale (EQ VAS) score, and/or a decrease from baseline of one or more of urticaria activity score (UAS), angioedema activity score (AAS), dermatology life quality index (DLQI), children’s dermatology life quality index (CDLQI), chronic urticaria quality of life questionnaire (CU-Q2oL), patient global impression of change (PGIC), and patient global impression of severity (PGIS). As used herein, the term “baseline,” with regard to a CSU-associated PRO measure, means the numerical value of the PRO measure for a patient prior to or at the time of administration of a pharmaceutical composition comprising an IL-4R antagonist.

To determine whether an CSU-associated parameter has “improved,” the parameter is quantified at baseline and at a time point after administration of the pharmaceutical composition described herein. For example, an CSU-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the CSU-associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as a CSU-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Itch-Free Days: According to certain embodiments, administration of an IL-4R antagonist to a patient results in an increase from baseline in itch-free days experienced by a subject. For example, administration of an IL-4R antagonist to a subject in need thereof causes an increase from baseline in itch-free days experienced by a subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Hive-Free Days: According to certain embodiments, administration of an IL-4R antagonist to a patient results in an increase from baseline in hive-free days experienced by a subject. For example, administration of an IL-4R antagonist to a subject in need thereof causes an increase from baseline in hive-free days experienced by a subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Itch Severity Score: According to certain embodiments, administration of an IL-4R antagonist to a patient results in a decrease from baseline of itch severity score (ISS). ISS7 is defined as the sum of the daily ISS scores (ranging from 0=none to 3=intense) recorded at the same time of the day for a 7-day period. The ISS7 ranges from 0-21, with higher scores indicating worse disease. The minimal important difference (MID) for ISS7 is 4.5-5.

Therapeutic methods are provided that result in a decrease in ISS7 score from baseline. For example, administration of an IL-4R antagonist to a subject in need thereof causes a decrease in ISS7 score from baseline of about 1, 2, 3, 4, 5, 6, 7, 8, 9, 10, 11, 12, 13, 14, 15, 16, 17, 18, 19, 20, or 21 points.

Hive Severity Score: According to certain embodiments, administration of an IL-4R antagonist to a patient results in a decrease from baseline of hive severity score (HSS). HSS7 is defined as the sum of the daily HSS scores (ranging from 0=none to 3= more than 50 hives) recorded at the same time of the day for a 7-day period. The HSS7 ranges from 0-21, with higher scores indicating worse disease. The minimal important difference (MID) for HSS7 is 5-5.5.

Therapeutic methods are provided that result in a decrease in HSS7 score from baseline. For example, administration of an IL-4R antagonist to a subject in need thereof causes a decrease in HSS7 score from baseline of about 1, 2, 3, 4, 5, 6, 7, 8, 9, 10, 11, 12, 13, 14, 15, 16, 17, 18, 19, 20, or 21 points.

Urticaria activity score: According to certain embodiments, administration of an IL-4R antagonist to a patient results in a decrease from baseline of urticaria activity score (UAS). UAS The Urticaria Activity Score (UAS) is a validated patient-recorded outcome (PRO) measure. The daily UAS is the sum of the daily Hive Severity Score (HSS, ranging from 0 = None to 3 = more than 50 hives) and the daily Itch Severity Score (ISS, ranging from 0 = None to 3 = intense), the 2 key urticaria signs and symptoms which are wheals and itch. The daily UAS scores range from 0 to 6 point/day. Daily UAS scores are summed over 7-day period to create the UAS7, ranging from 0 to 42, and is composed of the HSS7 and ISS7 components. The UAS7 is an established and widely accepted PRO tool to prospectively measure CSU activity. (See Mlynek A, et al. How to assess disease activity in patients with chronic urticaria? Allergy. 2008;63(6):777-80.) It has been used in most clinical trials in CSU in the recent years as a main outcome parameter and medical practice. (See Maurer M, et al. Omalizumab for the treatment of chronic idiopathic or spontaneous urticaria. N Engl J Med. 2013;368(10):924-35; Casale TB, et al. Similar efficacy with omalizumab in chronic idiopathic/spontaneous urticaria despite different background therapy. J Allergy Clin Immunol Pract. 2015;3(5):743-50). A minimal important difference (MID) value ranging from 9.5 to 10.5 has been defined to help interpretation of the change in score in CSU participants. (See Hollis K, et al. Comparison of urticaria activity score over 7 days (UAS7) values obtained from once-daily and twice-daily versions: Results from the ASSURE-CSU study. Am J Clin Dermatol. 2018;19(2):267-74; Hawro T, et al. The urticaria activity score-validity, reliability, and responsiveness. J Allergy Clin Immunol Pract. 2018;6(4):1185-90; Mathias SD, et al. Evaluating the minimally important difference of the urticaria activity score and other measures of disease activity in patients with chronic idiopathic urticaria. Ann Allergy Asthma Immunol. 2012;108(1):20-4.) The UAS7 ranges from 0-42, with higher scores indicating greater disease activity. Scores of 1-6 indicate well controlled urticaria. Scores of 7-15 indicate mild urticaria. Scores of 16-27 indicate moderate urticaria activity. Scores of 28-42 indicate severe urticaria activity. A UAS7 score of 6 or less is considered to indicate well controlled urticaria. A complete responder (no itch and no hives) has a UAS7 of 0.

Therapeutic methods are provided that result in a decrease in UAS or UAS7 score from baseline. For example, administration of an IL-4R antagonist to a subject in need thereof causes a decrease in UAS7 score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 points.

Angioedema Activity Score: According to certain embodiments, administration of an IL-4R antagonist to a patient results in a decrease from baseline of angioedema activity score (AAS). The angioedema activity score (AAS) is a validated PRO measure that assesses angioedema activity (See Weller K, et al. Development, validation, and initial results of the Angioedema Activity Score. Allergy. 2013;68(9):1185-92.) The AAS includes patients documenting the presence or absence of angioedema during the past 24 hours. If angioedema is present, patients answer 5 additional questions about the time of the day the swelling episode occurred, and the severity and impact on daily functioning and appearance this swelling episode has had. Each AAS item is scored between 0 and 3 points, that is, the minimum and maximum daily AASs are 0 and 15 points. The daily AASs are summed up to 7-day scores (AAS7), with 7-day scores ranging from 0 to 105 (Id). A MID of the AAS7 of around 8 points has been established (Id).

Therapeutic methods are provided that result in a decrease in AAS or AAS7 score from baseline. For example, administration of an IL-4R antagonist to a subject in need thereof causes a decrease in AAS or AAS7 score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, or 105 points.

Urticaria control test: According to certain embodiments, administration of an IL-4R antagonist to a patient results in an increase from baseline of the urticaria control test score. The urticaria control test (UCT) is a validated PRO measure for assessing urticaria control (Weller K, et al. Development, validation, and initial results of the Angioedema Activity Score. Allergy. 2013;68(9):1185-92.) based on 4 items (severity of pruritus and wheals urticaria symptoms; frequency of treatment being not sufficient; QoL impairment; overall urticarial control). Each item is rated on a 5-point Likert-type scale (scored with 0 to 4 points). Low scores indicate high disease activity and low disease control. The UCT total score is calculated by adding all 4 individual item scores. Accordingly, the minimum and maximum UCT scores are 0 and 16, with a score of 16 points indicating complete disease control (See Weller K, et al. Development, validation, and initial results of the Angioedema Activity Score. Allergy. 2013;68(9):1185-92.) A UCT score of 12 or greater indicates well controlled disease. The MID for UCT is 3.

Therapeutic methods are provided that result in an increase in UCT score from baseline. For example, administration of an IL-4R antagonist to a subject in need thereof causes an increase in UCT score from baseline of about 1, 2, 3, 4, 5, 6, 7, 8, 9, 10, 11, 12, 13, 14, 15, or 16 points.

Dermatology life quality index (DLQI): According to certain embodiments, administration of an IL-4R antagonist to a patient results in a decrease from baseline of the DLQI score. The Dermatology life quality index (DLQI) is a PRO developed to measure dermatology-specific HRQoL in adult participants (See Finlay AY, Khan GK. Dermatology life quality index (DLQI): a simple practical measure for routine clinical use. Clin Exp Dermatol.1994;19:210-6.) The instrument comprises 10 items assessing the impact of skin disease on participants’ health-related quality of life (HRQoL) over the previous week. The items cover symptoms, leisure activities, work/school or holiday time, personal relationships including intimate, the side effects of treatment, and emotional reactions to having a skin disease. It is a validated questionnaire used in clinical practice and clinical trials (See Chemyshov PV. The evolution of quality of life assessment and use in dermatology. Dermatology. 2019;235(3):167-74.) Response scale is a 4-point Likert scale (0 = “not at all” and 3 = “very much”) for 9 items. The remaining 1 item about work/studying asks whether work/study has been prevented and then (if “No”) to what degree the skin condition has been a problem at work/study; the item is rated on a 3-point Likert scale (‘Not at all’ to ‘A lot’). Overall scoring ranges from 0 to 30, with a high score indicative of a poor HRQoL. Using an integrated analysis of distribution and anchor-based approaches using the change in DLQI total score and participant-assessed itch severity scores, the MID for the DLQI in participants with chronic idiopathic urticaria was reported to be in the range of 2.24 to 3.10 points (Shikiar R, et al. Minimal important difference (MID) of the dermatology life quality index (DLQI): results from patients with chronic idiopathic urticaria. Health Qual. Life Outcomes. 2005; 3:36.)

Therapeutic methods are provided that result in a decrease in DLQI score from baseline. For example, administration of an IL-4R antagonist to a subject in need thereof causes a decrease in DLQI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Children’s Dermatology Quality Life Quality Index (CDLQI): According to certain embodiments, administration of an IL-4R antagonist to a patient results in a decrease from baseline of the CDLQI score. The Children’s Dermatology Quality Life Quality Index (CDLQI) is a validated questionnaire designed to measure the impact of skin disease on children’s HRQoL (See Lewis-Jones MS, Finlay AY. The children’s dermatology life quality index(CDLQI): initial validation and practical use. Br J Dermatol.1995;132(6):942-9.) Patients provide responses to 10 questions (symptoms feelings associated with disease, the impact of the disease on leisure, school or holidays, personal relationships, sleep, and side effects of treatment for the skin disease). The instrument has a recall period of 7 days. Nine of the 10 questions are scored on a 4-point Likert scale ranging from 0 = Not at all/question unanswered to 3 = Very much. Question 7 has an additional possible response (prevented school), which is assigned a score of 3. The CDLQI total score is the sum of the score of each question with a maximum of 30 and a minimum of 0. The higher the score, the greater the impact is on the child’s HRQoL. Patients complete the DLQI (≥16 years old) or CDLQI (≥12 - <16).

Therapeutic methods are provided that result in a decrease in CDLQI score from baseline. For example, administration of an IL-4R antagonist to a subject in need thereof causes a decrease in CDLQI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Chronic urticaria quality of life questionnaire (CU-Q2oL): According to certain embodiments, administration of an IL-4R antagonist to a patient results in a decrease from baseline of the CU-Q2oL score. The CU-Q2oL is a disease-specific instrument used to assess the QoL in adult participants with CSU. (See Baiardini I, et al. A new tool to evaluate the impact of chronic urticaria on quality of life: chronic urticaria quality of life questionnaire (CU-QoL). Allergy. 2005;60(8):1073-8.) The CU-Q2oL is a 23-item, self-administered questionnaire that includes 6 QoL dimensions: pruritus, swelling, impact on life activities, sleep problems, limits, and looks. Each item is scored on a 5-point Likert scale (1 = not at all, 5 = extremely) where participants indicate how troubled they are within each dimension. The individual items are summed to generate the overall CU-Q2oL score, which is then converted to a 0 to 100 scale; higher scores indicate greater QoL impairment.

Therapeutic methods are provided that result in a decrease in CU-Q2oL score from baseline. For example, administration of an IL-4R antagonist to a subject in need thereof causes a decrease in CU-Q2oL score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

Patient Global Impression of Change (PGIC): According to certain embodiments, administration of an IL-4R antagonist to a patient results in a decrease from baseline of PGIC score. The Patient Global Impression of Change (PGIC) is a 1-item questionnaire that asks the participant to provide the overall self-assessment of change in their CSU on a 7-point scale, compared to just before participant started taking the study treatment. Response choices are: 0 = “very much better,” 1 = “moderately better,” 2 = “a little better,” 3 = “no change,” 4 = “a little worse,” 5 = “moderately worse,” 6 = “very much worse.” (See Guy W et al. ECDEU Assessment Manual for Psychopharmacology. Rockville, MD: US Department of Health, Education, and Welfare Public Health Service Alcohol, Drug Abuse, and Mental Health Administration, 1976.)

Therapeutic methods are provided that result in a decrease in PGIC score from baseline. For example, administration of an IL-4R antagonist to a subject in need thereof causes a decrease in PGIC score from baseline of about 1, 2, 3, 4, 5, or 6.

Patient Global Impression of Severity (PGIS): According to certain embodiments, administration of an IL-4R antagonist to a patient results in a decrease from baseline of PGIS score. The Patient Global Impression of Severity (PGIS) is a 1-item questionnaire that asks participants to provide the overall self-assessment of their participant’s disease severity on a 4-point scale for the past week. Response choices are: 1 = “none,” 2 = “mild,” 3 = “moderate,” 4 = “severe.” (See Guy W et al. ECDEU Assessment Manual for Psychopharmacology. Rockville, MD: US Department of Health, Education, and Welfare Public Health Service Alcohol, Drug Abuse, and Mental Health Administration, 1976.)

Therapeutic methods are provided that result in a decrease in PGIS score from baseline. For example, administration of an IL-4R antagonist to a subject in need thereof causes a decrease in PGIS score from baseline of about 1, 2, or 3.

Euroqol-5 dimensions (EQ-5D) and EQ-5D Youth version (EQ-5D Y): According to certain embodiments, administration of an IL-4R antagonist to a patient results in an increase from baseline of EQ-5D or EQ-5D Y score. The Euroqol-5 dimensions (EQ-5D) is a standardized PRO measure of health status developed by the EuroQol Group in order to provide a simple, generic measure of health for clinical and economic appraisal. The adult version of the questionnaire is adapted to patients aged 16 and older. The EQ-5D consists of 2 parts: the descriptive system and the EQ visual analogue scale (EQ VAS). The EQ-5D 5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “inability to do the activity.” (See Herdman M, et al. Development and preliminary testing of the new five-level version of EQ-5D (EQ-5D-5L). Qual. Life Res. 2011;20(10):1727-36.) The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent’s health state. The EQ VAS records the respondent’s self-rated health on a vertical, VAS where the endpoints are labeled “best imaginable health state (100)” and “worst imaginable health state (0).” This information can be used as a quantitative measure of health outcome as judged by the individual respondents. The EQ-5D Youth version (EQ-5D Y) is administered to children ≥6 to <12 years old and adolescents 12 to 15 years old. (Wille N, et al. Qual. Life Res. 2010;19(6):875-86.) The EQ-5D-Y is based on the EQ-5D-3L and essentially consists of 2 pages: the EQ-5D descriptive system and the EQ VAS. The EQ-5D-Y descriptive system comprises the following 5 dimensions: mobility, looking after myself, doing usual activities, having pain or discomfort and feeling worried, sad or unhappy. Each dimension has 3 levels: no problems, some problems and a lot of problems. The EQ VAS records the younger patient’s self-rated health on a vertical VAS where the endpoints are labelled “The best health you can imagine” and “The worst health you can imagine.” Patients complete the EQ-5D Y or EQ-5D questionnaire.

Therapeutic methods are provided that result in an increase in EQ VAS score from baseline. For example, administration of an IL-4R antagonist to a subject in need thereof causes an increase in EQ VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

### Children’s Dermatology Quality Life Quality Index (C-DLQI)

The C-DLQI is a validated questionnaire designed to measure the impact of skin disease on children’s Health-Related Quality of Life (HRQoL) HRQoL. The questionnaire is validated for children aged ≥4 to <16 years. The C-DLQI is recommended for children aged 4 to 12, participants should complete the questionnaire themselves, however, younger children may complete the questionnaire with assistance from their parent/caregiver. Participants provide responses to 10 questions (symptoms feelings associated with disease, the impact of the disease on leisure, school or holidays, personal relationships, sleep, and side effects of treatment for the skin disease). The instrument has a recall period of 1 week (7 days). Nine of the 10 questions are scored on a 4-point Likert Scale ranging from 0 = Not at all/question unanswered to 3 = Very much. Question 7 has an additional possible response (prevented school), which is assigned a score of 3. The C-DLQI total score is the sum of the score of each question with a maximum of 30 and a minimum of 0. The higher the score, the greater the impact is on the child’s HRQoL.

### Infants' Dermatitis Quality of Life Index (IDQOL)

The IDQOL is a validated questionnaire designed for use in children aged <4 years. The questionnaire is completed by the child’s caregiver/guardian. The instrument has a recall period of 1 week (7 days). There are 11 questions in total, 10 focusing on the topic of Life Quality Index scored on a 4-point Likert Scale plus an additional question scored on a 5-point Likert Scale focusing on Dermatitis Severity. For the Life Quality Index, questions 1, 5 - 10, the scoring is: all the time = 3 to none = 0. For question 2, the scoring is: always crying, etc. = 3, very fretful = 2, slightly fretful = 1, happy = 0. For question 3, the scoring is: more than 2 hours = 3, 1 to 2 hours = 2, 15 minutes to 1 hour = 1, 0 to 15 minutes = 0. For question 4, the scoring is: 5 hours or more = 3, 3 to 4 hours = 2, 1 to 2 hours = 1, Less than 1 hour = 0. For the dermatitis severity, the 5-point Likert Scale scoring is: extremely severe = 4 to none = 0. The IDQOL total score is the sum of the score of each question with a maximum of 30 and a minimum of 0. The higher the score, the greater the impact is on the child’s HRQoL.

### Modified Urticaria Activity Score (UAS)

The Urticaria Activity Score (UAS) is a validated patient reported outcome (PRO) measure. A modified version of the UAS (mUAS) will be used in this study to account for the smaller body surface area of child and adolescent patients. The mUAS is derived from the sum of the daily Hive Severity Score (HSS, ranging from 0 to 3 (0 = absent, 1 = mild: (1 to <10 wheals/24 hours); 2 = moderate: (10 to 30 wheals/24 hours); and 3 = intense: (>30 wheals/24 hours or large confluent areas of wheals) and the daily Itch Severity Score (ISS, ranging from 0 = None to 3 = intense). Wheals and itching are the 2 key symptoms in urticaria. The daily mUAS total scores range from 0 to 6 (0 to 3 for the Itch Severity Score and 0 to 3 for the Hive Severity Score). Daily mUAS scores are summed over 7 day period to create the UAS7, ranging from 0 to 42, and is composed of the hive severity score over 7 days (HSS7) and itch severity score over 7 days (ISS7) components. Completion of mUAS7 should be done by the child or parent(s)/caregiver(s)/legal guardian(s) for participants aged 4 years or older; and by parent(s)/caregiver(s) for participants aged less than 4 years.

The UAS7 is an established and widely accepted PRO tool to prospectively measure CSU activity. A minimal important difference (MID) value ranging from 9.5 to 10.5 has been defined to help interpretation of the change in score in CSU participants.

The itch severity score (ISS) is a single item scale scored on a 0 to 3 Likert Scale ranging from 0 = None to 3 = intense. The ISS will be collected daily and used to derive the mUAS as described above.

The hive severity score (HSS) is a single-item scale scored on a 0 to 3 Likert Scale ranging from 0 = Absent to 3 = intense. The HSS will be collected daily and used to derive the mUAS score as described above.

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R, and comprises the heavy chain and light chain (Complementarity Determining Region) CDR sequences from the Heavy Chain Variable Region (HCVR) and Light Chain Variable Region (LCVR) of SEQ ID NOs: 1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab')2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3—CDR3—FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}—C_{H}1; (ii) V_{H}—C_{H}2; (iii) V_{H}—C_{H}3; (iv) V_{H}—C_{H}1—C_{H}2; (v) V_{H}—C_{H}1—C_{H}2—C_{H}3; (vi) V_{H}—C_{H}2—C_{H}3; (vii) V_{H}—C_{L}; (viii) V_{L}—C_{H}1; (ix) V_{L}—C_{H}2; (x) V_{L}—C_{H}3; (xi) V_{L}—C_{H}1—C_{H}2; (xii) V_{L}—C_{H}1—C_{H}2—C_{H}3; (xiii) V_{L}—C_{H}2—C_{H}3; and (xiv) V_{L}—C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the disclosure.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24: 307-331, herein incorporated by reference.) Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild-type protein and a mutein thereof. (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, US 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully-human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is dupilumab, which comprises the HCVR/LCVR amino acid sequence pair of SEQ ID NOs: 1 and 2.

In certain embodiments, the antibody sequence is dupilumab, which comprises the heavy chain/light chain amino acid sequence pair of SEQ ID NOs: 9 and 10.

### Dupilumab HCVR Amino Acid Sequence

EVQLVESGGGLEQPGGSLRLSCAGSGFTFRDYAMTWVRQAPGKGLEWVSSISG SGGNTYYADSVKGRFTISRDNSKNTLYLQMNSLRAEDTAVYYCAKDRLSITIRPRYYGL DVWGQGTTVTVS (SEQ ID NO: 1).

### Dupilumab LCVR Amino Acid Sequence

DIVMTQSPLSLPVTPGEPASISCRSSQSLLYSIGYNYLDWYLQKSGQSPQLLIYLG SNRASGVPDRFSGSGSGTDFTLKISRVEAEDVGFYYCMQALQTPYTFGQGTKLEIK (SEQ ID NO: 2).

### Dupilumab HCDR1 Amino Acid Sequence

GFTFRDYA (SEQ ID NO: 3).

### Dupilumab HCDR2 Amino Acid Sequence

ISGSGGNT (SEQ ID NO: 4).

### Dupilumab HCDR3 Amino Acid Sequence

AKDRLSITIRPRYYGL (SEQ ID NO: 5).

### Dupilumab LCDR1 Amino Acid Sequence

QSLLYSIGYNY (SEQ ID NO: 6).

### Dupilumab LCDR2 Amino Acid Sequence

LGS.

### Dupilumab LCDR3 Amino Acid Sequence

MQALQTPYT (SEQ ID NO: 8).

### Dupilumab HC Amino Acid Sequence

EVQLVESGGGLEQPGGSLRLSCAGSGFTFRDYAMTWVRQAPGKGLEWVSSISG SGGNTYYADSVKGRFTISRDNSKNTLYLQMNSLRAEDTAVYYCAKDRLSITIRPRYYGL DVWGQGTTVTVSSASTKGPSVFPLAPCSRSTSESTAALGCLVKDYFPEPVTVSWNSGAL TSGVHTFPAVLQSSGLYSLSSVVTVPSSSLGTKTYTCNVDHKPSNTKVDKRVESKYGPP CPPCPAPEFLGGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSDG SFFLYSRLTVDKSRWQEGNVFSCSVMHEALHNHYTQKSLSLSLG (SEQ ID NO: 9) (amino acids 1-124 = HCVR; amino acids 125-451 = HC constant).

### Dupilumab LC Amino Acid Sequence

DIVMTQSPLSLPVTPGEPASISCRSSQSLLYSIGYNYLDWYLQKSGQSPQLLIYLG SNRASGVPDRFSGSGSGTDFTLKISRVEAEDVGFYYCMQALQTPYTFGQGTKLEIKRTV AAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO: 10) (amino acids 1-112 = LCVR; amino acids 112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39 / SCB-VH-92; SCB-VL-40 / SCB-VH-92; SCB-VL-41 / SCB-VH-92; SCB-VL-42 / SCB-VH-92; SCB-VL-43 / SCB-VH-92; SCB-VL-44 / SCB-VH-92; SCB-VL-44 / SCB-VH-62; SCB-VL-44 / SCB-VH-68; SCB-VL-44 / SCB-VH-72; SCB-VL-44 / SCB-VH-82; SCB-VL-44 / SCB-VH-85; SCB-VL-44 / SCB-VH-91; SCB-VL-44 / SCB-VH-93; SCB-VL-45 / SCB-VH-92; SCB-VL-46 / SCB-VH-92; SCB-VL-47 / SCB-VH-92; SCB-VL-48 /SCB-VH-92; SCB-VL-49 / SCB-VH-92; SCB-VL-50 / SCB-VH-92; SCB-VL-51 / SCB-VH-92; SCB-VL-51 / SCB-VH-93; SCB-VL-52 / SCB-VH-92; SCB-VL-52 / SCB-VH-62; SCB-VL-52 / SCB-VH-91; SCB-VL-53 / SCB-VH-92; SCB-VL-54 / SCB-VH-92; SCB-VL-54 / SCB-VH-62; SCB-VL-54 / SCB-VH-68; SCB-VL-54 / SCB-VH-72; SCB-VL-54 / SCB-VH-82; SCB-VL-54 / SCB-VH-85; SCB-VL-54 / SCB-VH-91; SCB-VL-55 / SCB-VH-92; SCB-VL-55 / SCB-VH-62; SCB-VL-55 / SCB-VH-68; SCB-VL-55 / SCB-VH-72; SCB-VL-55 / SCB-VH-82; SCB-VL-55 / SCB-VH-85; SCB-VL-55 / SCB-VH-91; SCB-VL-56 / SCB-VH-92; SCB-VL-57 / SCB-VH-92; SCB-VL-57 / SCB-VH-93; SCB-VL-57 / SCB-VH-59; SCB-VL-57 / SCB-VH-60; SCB-VL-57 / SCB-VH-61; SCB-VL-57 / SCB-VH-62; SCB-VL-57 / SCB-VH-63; SCB-VL-57 / SCB-VH-64; SCB-VL-57 / SCB-VH-65; SCB-VL-57 / SCB-VH-66; SCB-VL-57 / SCB-VH-67; SCB-VL-57 / SCB-VH-68; SCB-VL-57 / SCB-VH-69; SCB-VL-57 / SCB-VH-70; SCB-VL-57 / SCB-VH-71; SCB-VL-57 / SCB-VH-72; SCB-VL-57 / SCB-VH-73; SCB-VL-57 / SCB-VH-74; SCB-VL-57 / SCB-VH-75; SCB-VL-57 / SCB-VH-76; SCB-VL-57 / SCB-VH-77; SCB-VL-57 / SCB-VH-78; SCB-VL-57 / SCB-VH-79; SCB-VL-57 / SCB-VH-80; SCB-VL-57 / SCB-VH-81; SCB-VL-57 / SCB-VH-82; SCB-VL-57 / SCB-VH-83; SCB-VL-57 / SCB-VH-84; SCB-VL-57 / SCB-VH-85; SCB-VL-57 / SCB-VH-86; SCB-VL-57 / SCB-VH-87; SCB-VL-57 / SCB-VH-88; SCB-VL-57 / SCB-VH-89; SCB-VL-57 / SCB-VH-90; SCB-VL-57 / SCB-VH-91; SCB-VL-58 / SCB-VH-91; SCB-VL-58 / SCB-VH-92; and SCB-VL-58 / SCB-VH-93.

In certain embodiments, an antibody or antigen-binding fragment thereof of the disclosure comprises a LCVR / HCVR sequence pair of SCB-VL-44 / SCB-VH-92.

In certain embodiments, an antibody or antigen-binding fragment thereof of the disclosure comprises a LCVR / HCVR sequence pair of SCB-VL-54 / SCB-VH-92.

In certain embodiments, an antibody or antigen-binding fragment thereof of the disclosure comprises a LCVR / HCVR sequence pair of SCB-VL-55 / SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. 10,774,141, incorporated herein by reference in its entirety for all purposes.

TABLE 1

| | |
|---|---|
| Sequence ID | Sequence |
| SCB-VL-39 | EIVLTQSPGTLSLSPGERATLSCRASQSVSNSYLAWYQQKPGQAPRLL IFGASSRATGIPDRFSGSGSGTDFTLTISRLEPEDFAVYYCQQYGSSPP WTFGQGTKVEIK (SEQ ID NO: 11) |
| SCB-VL-40 | EIVLTQSPGTLSLSPGERATLSCRASQSVSSSYLAWYQQKPGQAPRLL IYGASSRATGIPDRFSGSGSGTDFTLTISRLEPEDFAVYYCQQYGSSPP WTFGQGTKVEIK (SEQ ID NO: 12) |
| SCB-VL-41 | EIVLTQSPGTLSLSPGERATLSCRASQSVSSSYLAWYQQKPGQAPRLL IFGASSRAPGIPDRFSGSGSGTDFTLTISRLEPEDFAVYYCQQYGSSPP WTFGQGTKVEIK (SEQ ID NO: 13) |
| SCB-VL-42 | EIVLTQSPGTLSLSPGERATLSCRASQSVSNSYLAWYQQKPGQAPRLL IYGASSRATGIPDRFSGSGSGTDFTLTISRLEPEDFAVYYCQQYGSSPP WTFGQGTKVEIK (SEQ ID NO: 14) |
| SCB-VL-43 | EIVLTQSPGTLSLSPGERATLSCRASQSVSNSYLAWYQQKPGQAPRLL IFGASSRAPGIPDRFSGSGSGTDFTLTISRLEPEDFAVYYCQQYGSSPP WTFGQGTKVEIK (SEQ ID NO: 15) |
| SCB-VL-44 | EIVLTQSPGTLSLSPGERATLSCRASQSVSSSYLAWYQQKPGQAPRLL IYGASSRAPGIPDRFSGSGSGTDFTLTISRLEPEDFAVYYCQQYGSSPP WTFGQGTKVEIK (SEQ ID NO: 16) |
| SCB-VL-45 | EIVLTQSPGTLSLSPGERATLSCRASQSVSSSYLAWYQQKPGQAPRLL IFGASSRATGIPDRFSGSGSGTDFTLTISRLEPEDFAVYYCQQYDHSPP WTFGQGTKVEIK (SEQ ID NO: 17) |
| SCB-VL-46 | EIVLTQSPGTLSLSPGERATLSCRASQSVSSSYLAWYQQKPGQAPRLL IFGAS SRATGIPDRF SGSGSGTDFTLTISRLEPEDFAVYYCQQYGS SAG WTFGQGTKVEIK (SEQ ID NO: 18) |
| SCB-VL-47 | EIVLTQSPGTLSLSPGERATLSCRASQSVSSSYLAWYQQKPGQAPRLL IFGASSRATGIPDRFSGSGSGTDFTLTISRLEPEDFAVYYCQQYDHSAG WTFGQGTKVEIK (SEQ ID NO: 19) |
| SCB-VL-48 | EIVLTQSPGTLSLSPGERATLSCRASQSVSNSYLAWYQQKPGQAPRLL IFGASSRATGIPDRFSGSGSGTDFTLTISRLEPEDFAVYYCQQYDHSPP WTFGQGTKVEIK (SEQ ID NO: 20) |
| SCB-VL-49 | EIVLTQSPGTLSLSPGERATLSCRASQSVSSSYLAWYQQKPGQAPRLL IYGASSRATGIPDRFSGSGSGTDFTLTISRLEPEDFAVYYCQQYDHSPP WTFGQGTKVEIK (SEQ ID NO: 21) |
| SCB-VL-50 | EIVLTQSPGTLSLSPGERATLSCRASQSVSSSYLAWYQQKPGQAPRLL IFGASSRAPGIPDRFSGSGSGTDFTLTISRLEPEDFAVYYCQQYDHSPP WTFGQGTKVEIK (SEQ ID NO: 22) |
| SCB-VL-51 | EIVLTQSPGTLSLSPGERATLSCRASQSVSSSYLAWYQQKPGQAPRLL IYGASSRAPGIPDRFSGSGSGTDFTLTISRLEPEDFAVYYCQQYDHSA GWTFGQGTKVEIK (SEQ ID NO: 23) |
| SCB-VL-52 | EIVLTQSPGTLSLSPGERATLSCRASQSVSNSYLAWYQQKPGQAPRLL IFGASSRAPGIPDRFSGSGSGTDFTLTISRLEPEDFAVYYCQQYDHSAG WTFGQGTKVEIK (SEQ ID NO: 24) |
| SCB-VL-53 | EIVLTQSPGTLSLSPGERATLSCRASQSVSNSYLAWYQQKPGQAPRLL IYGASSRATGIPDRFSGSGSGTDFTLTISRLEPEDFAVYYCQQYDHSA GWTFGQGTKVEIK (SEQ ID NO: 25) |
| SCB-VL-54 | EIVLTQSPGTLSLSPGERATLSCRASQSVSSSYLAWYQQKPGQAPRLL IFGASSRAPGIPDRFSGSGSGTDFTLTISRLEPEDFAVYYCQQYDHSAG WTFGQGTKVEIK (SEQ ID NO: 26) |
| SCB-VL-55 | EIVLTQSPGTLSLSPGERATLSCRASQSVSSSYLAWYQQKPGQAPRLL IYGASSRATGIPDRFSGSGSGTDFTLTISRLEPEDFAVYYCQQYDHSA GWTFGQGTKVEIK (SEQ ID NO: 27) |
| SCB-VL-56 | EIVLTQSPGTLSLSPGERATLSCRASQSVSNSYLAWYQQKPGQAPRLL IFGASSRATGIPDRFSGSGSGTDFTLTISRLEPEDFAVYYCQQYDHSAG WTFGQGTKVEIK (SEQ ID NO: 28) |
| SCB-VL-57 | EIVLTQSPGTLSLSPGERATLSCRASQSVSSSYLAWYQQKPGQAPRLL IFGASSRATGIPDRFSGSGSGTDFTLTISRLEPEDFAVYYCQQYGSSPP WTFGQGTKVEIK (SEQ ID NO: 29) |
| SCB-VL-58 | EIVLTQSPGTLSLSPGERATLSCRASQSVSNSYLAWYQQKPGQAPRLL IYGASSRAPGIPDRFSGSGSGTDFTLTISRLEPEDFAVYYCQQYDHSA GWTFGQGTKVEIK (SEQ ID NO: 30) |
| SCB-VH-59 | EVQLVESGGGLVHPGGSLRLSCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 31) |
| SCB-VH-60 | EVQLVQSGGGLVQPGGSLRLSCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 32) |
| SCB-VH-61 | EVQLVQSGGGLVHPGGSLRLSCAASGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 33) |
| SCB-VH-62 | EVQLVQSGGGLVHPGGSLRLSCAGSGFTFSRNAMFWVRQAPGKGLE WVSGIGTGGATSYADSVKGRFTISRDNAKNSLYLQMNSLRAEDMAV YYCARGRYYFDYWGQGTLVTVSS (SEQ ID NO: 34) |
| SCB-VH-63 | EVQLVQSGGGLVHPGGSLRLSCAGSGFTFSRNAMFWVRQAPGKGLE WVSGIGTGGATNYADSVKGRFTISRDNAKNSLYLQMNSLRAEDTAV YYCARGRYYFDYWGQGTLVTVSS (SEQ ID NO: 35) |
| SCB-VH-64 | EVQLVESGGGLVQPGGSLRLSCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 36) |
| SCB-VH-65 | EVQLVESGGGLVHPGGSLRLSCAASGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 37) |
| SCB-VH-66 | EVQLVQSGGGLVQPGGSLRLSCAASGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 38) |
| SCB-VH-67 | EVQLVQSGGGLVHPGGSLRLSCAGSGFTFSRNAMFWVRQAPGKGLE WVSGIGTGGATSYADSVKGRFTISRDNAKNSLYLQMNSLRAEDTAV YYCARGRYYFDYWGQGTLVTVSS (SEQ ID NO: 39) |
| SCB-VH-68 | EVQLVQSGGGLVHPGGSLRLSCAGSGFTFSRNAMFWVRQAPGKGLE WVSGIGTGGATNYADSVKGRFTISRDNAKNSLYLQMNSLRAEDMA VYYCARGRYYFPWWGQGTLVTVSS (SEQ ID NO: 40) |
| SCB-VH-69 | EVQLVESGGGLVHPGGSLRLSCAGSGFTFSRNAMFWVRQAPGKGLE WVSGIGTGGATNYADSVKGRFTISRDNAKNSLYLQMNSLRAEDMA VYYCARGRYYFPWWGQGTLVTVSS (SEQ ID NO: 41) |
| SCB-VH-70 | EVQLVQSGGGLVQPGGSLRLSCAGSGFTFSRNAMFWVRQAPGKGLE WVSGIGTGGATNYADSVKGRFTISRDNAKNSLYLQMNSLRAEDMA VYYCARGRYYFPWWGQGTLVTVSS (SEQ ID NO: 42) |
| SCB-VH-71 | EVQLVQSGGGLVHPGGSLRLSCAASGFTFSRNAMFWVRQAPGKGLE WVSGIGTGGATNYADSVKGRFTISRDNAKNSLYLQMNSLRAEDMA VYYCARGRYYFPWWGQGTLVTVSS (SEQ ID NO: 43) |
| SCB-VH-72 | EVQLVQSGGGLVHPGGSLRLSCAGSGFTFSRNAMFWVRQAPGKGLE WVSGIGTGGATSYADSVKGRFTISRDNAKNSLYLQMNSLRAEDMAV YYCARGRYYFPWWGQGTLVTVSS (SEQ ID NO: 44) |
| SCB-VH-73 | EVQLVQSGGGLVHPGGSLRLSCAGSGFTFSRNAMFWVRQAPGKGLE WVSGIGTGGATNYADSVKGRFTISRDNAKNSLYLQMNSLRAEDTAV YYCARGRYYFPWWGQGTLVTVSS (SEQ ID NO: 45) |
| SCB-VH-74 | EVQLVQSGGGLVHPGRSLRLSCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 46) |
| SCB-VH-75 | EVQLVQSGGGLVHPGGSLRLTCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 47) |
| SCB-VH-76 | EVQLVQSGGGLVHPGGSLRLSCAGSGFTFSRNAMHWVRQAPGKGL EWVSGIGTGGATNYADSVKGRFTISRDNAKNSLYLQMNSLRAEDMA VYYCARGRYYFDYWGQGTLVTVSS (SEQ ID NO: 48) |
| SCB-VH-77 | EVQLVQSGGGLVHPGGSLRLSCAGSGFTFSRNAMFWVRQAPGEGLE WVSGIGTGGATNYADSVKGRFTISRDNAKNSLYLQMNSLRAEDMA VYYCARGRYYFDYWGQGTLVTVSS (SEQ ID NO: 49) |
| SCB-VH-78 | EVQLVQSGGGLVHPGGSLRLSCAGSGFTFSRNAMFWVRQAPGKGLE WVSGIGTGGATNYADSVKGRFTISRDEAKNSLYLQMNSLRAEDMAV YYCARGRYYFDYWGQGTLVTVSS (SEQ ID NO: 50) |
| SCB-VH-79 | EVQLVQSGGGLVHPGGSLRLSCAGSGFTFSRNAMFWVRQAPGKGLE WVSGIGTGGATNYADSVKGRFTISRDNAKNSLYLQMNSLRAGDMA VYYCARGRYYFDYWGQGTLVTVSS (SEQ ID NO: 51) |
| SCB-VH-80 | EVQLVQSGGGLVHPGGSLRLSCAGSGFTFDDYAMFWVRQAPGKGL EWVSGIGTGGATNYADSVKGRFTISRDNAKNSLYLQMNSLRAEDMA VYYCARGRYYFDYWGQGTLVTVSS (SEQ ID NO: 52) |
| SCB-VH-81 | EVQLVQSGGGLVQPGGSLRLSCAASGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 53) |
| SCB-VH-82 | EVQLVESGGGLVHPGGSLRLSCAASGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 54) |
| SCB-VH-83 | EVQLVESGGGLVQPGGSLRLSCAGSGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 55) |
| SCB-VH-84 | EVQLVESGGGLVQPGGSLRLSCAASGFTFSRNAMFWVRQAPGKGLE WVSGIGTGGATNYADSVKGRFTISRDNAKNSLYLQMNSLRAEDTAV YYCARGRYYFPWWGQGTLVTVSS (SEQ ID NO: 56) |
| SCB-VH-85 | EVQLVESGGGLVQPGGSLRLSCAASGFTFSRNAMFWVRQAPGKGLE WVSGIGTGGATSYADSVKGRFTISRDNAKNSLYLQMNSLRAEDMAV YYCARGRYYFPWWGQGTLVTVSS (SEQ ID NO: 57) |
| SCB-VH-86 | EVQLVQSGGGLVHPGGSLRLSCAASGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 58) |
| SCB-VH-87 | EVQLVQSGGGLVQPGGSLRLSCAGSGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 59) |
| SCB-VH-88 | EVQLVESGGGLVHPGGSLRLSCAGSGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 60) |
| SCB-VH-89 | EVQLVQSGGGLVHPGGSLRLSCAGSGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 61) |
| SCB-VH-90 | EVQLVESGGGLVQPGGSLRLSCAASGFTFSRNAMFWVRQAPGKGLE WVSGIGTGGATNYADSVKGRFTISRDNAKNSLYLQMNSLRAEDMA VYYCARGRYYFPWWGQGTLVTVSS (SEQ ID NO: 62) |
| SCB-VH-91 | EVQLVESGGGLVQPGGSLRLSCAASGFTFSRNAMFWVRQAPGKGLE WVSGIGTGGATSYADSVKGRFTISRDNAKNSLYLQMNSLRAEDTAV YYCARGRYYFDYWGQGTLVTVSS (SEQ ID NO: 63) |
| SCB-VH-92 | EVQLVQSGGGLVHPGGSLRLSCAGSGFTFSRNAMFWVRQAPGKGLE WVSGIGTGGATNYADSVKGRFTISRDNAKNSLYLQMNSLRAEDMA VYYCARGRYYFDYWGQGTLVTVSS (SEQ ID NO: 64) |
| SCB-VH-93 | EVQLVESGGGLVQPGGSLRLSCAASGFTFSRNAMFWVRQAPGKGLE WVSGIGTGGATSYADSVKGRFTISRDNAKNSLYLQMNSLRAEDTAV YYCARGRYYFPWWGQGTLVTVSS (SEQ ID NO: 65) |
| SCB-92-HCDR1 | RNAMF (SEQ ID NO: 66) |
| SCB-92-HCDR3 | GIGTGGATSYADSVKG (SEQ ID NO: 67) |
| SCB-92-HCDR3 | GRYYFDY (SEQ ID NO: 68) |
| SCB-55-LCDR1 | RASQSVSSSYLA (SEQ ID NO: 69) |
| SCB-55-LCDR2 | GASSRAT (SEQ ID NO: 70) |
| SCB-55-LCDR3 | QQYDHSAGWT (SEQ ID NO: 71) |
| SCB-54-LCDR2 | GASSRAP (SEQ ID NO: 72) |
| SCB-44-LCDR3 | QQYGSSPPWT (SEQ ID NO: 73) |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL / MEDI-1-VH through MEDI-42-VL / MEDI-42-VH.

In certain embodiments, an antibody or antigen-binding fragment thereof of the disclosure comprises a LCVR / HCVR sequence pair of MEDI-37GL-VL / MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. 8,877,189, incorporated herein by reference in its entirety for all purposes.

TABLE 2

| | |
|---|---|
| Sequence ID | Sequence |
| MEDI-1-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLDYWGKGTLVTVSS (SEQ ID NO: 74) |
| MEDI-1-VL, | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SLSANYVFGTGTKLTVL (SEQ ID NO: 75) |
| MEDI-2-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-2-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SQPPNPLFGTGTKLTVL (SEQ ID NO: 77) |
| MEDI-3-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKLLKNPWGKGTLVTVSS (SEQ ID NO: 78) |
| MEDI-3-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWFG TPASNYVFGTGTKLTVL (SEQ ID NO: 79) |
| MEDI-4-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-4-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SSPPQPIFGTGTKLTVL (SEQ ID NO: 80) |
| MEDI-5-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYDWGKGTLVTVSS (SEQ ID NO: 81) |
| MEDI-5-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SSPPQPIFGTGTKLTVL (SEQ ID NO: 80) |
| MEDI-6-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-6-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTYHPIFGTGTKLTVL (SEQ ID NO: 83) |
| MEDI-7-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWWQYWGKGTLVTVSS (SEQ ID NO: 84) |
| MEDI-7-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SSPPQPIFGTGTKLTVL (SEQ ID NO: 80) |
| MEDI-8-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWWQYWGKGTLVTVSS (SEQ ID NO: 84) |
| MEDI-8-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTYHPIFGTGTKLTVL (SEQ ID NO: 83) |
| MEDI-9-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-9-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTMYPLFGTGTKLTVL (SEQ ID NO: 85) |
| MEDI-10-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYDWGKGTLVTVSS (SEQ ID NO: 81) |
| MEDI-10-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STVLTPIFGTGTKLTVL (SEQ ID NO: 86) |
| MEDI-11-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWFYDWGKGTLVTVSS (SEQ ID NO: 87) |
| MEDI-11-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SPSMIPLFGTGTKLTVL (SEQ ID NO: 88) |
| MEDI-12-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWFYDWGKGTLVTVSS (SEQ ID NO: 87) |
| MEDI-12-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTMYPLFGTGTKLTVL (SEQ ID NO: 85) |
| MEDI-13-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYDWGKGTLVTVSS (SEQ ID NO: 81) |
| MEDI-13-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTLQPLFGTGTKLTVL (SEQ ID NO: 89) |
| MEDI-14-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-14-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SPPTKPLFGTGTKLTVL (SEQ ID NO: 90) |
| MEDI-15-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-15-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STHRHPLFGTGTKLTVL (SEQ ID NO: 91) |
| MEDI-16-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWLYNWGKGTLVTVSS (SEQ ID NO: 76) |
| MEDI-16-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTYHPIFGTGTKLTVL (SEQ ID NO: 83) |
| MEDI-17-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWWQHWGKGTLVTVSS (SEQ ID NO: 92) |
| MEDI-17-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SPVDRPIFGTGTKLTVL (SEQ ID NO: 93) |
| MEDI-18-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWWQHWGKGTLVTVSS (SEQ ID NO: 92) |
| MEDI-18-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTPMPVFGTGTKLTVL (SEQ ID NO: 94) |
| MEDI-19-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKWWWQHWGKGTLVTVSS (SEQ ID NO: 92) |
| MEDI-19-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STTYHPIFGTGTKLTVL (SEQ ID NO: 83) |
| MEDI-20-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-20-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STVWEWPFGTGTKLTVL (SEQ ID NO: 95) |
| MEDI-21-VH | QVQLVQSGAEVKKPGASVKVSCKASGYAFTSYYMHWARQAPGQG LEWMGIINPSGGSASYAQKFQGRVTMTRDTSTSTVYMELSSLRSED TAVYYCARGKYWMYDWGKGTLVTVSS (SEQ ID NO: 96) |
| MEDI-21-VL | QSVLTQPPSVSAAPGQKVTISCSGGSSNIGNSYVSWYQQLPGTAPKL LIYDNNKRPSGIPDRFSGSKSGTSATLAITGLQTGDEAVYFCGTWDT STVWEWPFGTGTKLTVL (SEQ ID NO: 97) |
| MEDI-22-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-22-VL | QPVLTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 98) |
| MEDI-23-VH | QVQLVQSGAEVRKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 99) |
| MEDI-23-VL | QSVLTQPPSVSAAPGQKVTISCSGGSSNIGNNYVSWYQQLPGTAPKL LIYDNNKRPPGIPDRFSGSKSGTSATLAITGLQTGDEADYYCGTWDT STVWEWPFGTGTKLTVL (SEQ ID NO: 100) |
| MEDI-24-VH | QVQLVQSGAEVKKPGASVKVSCKASGYAFTSYYMHWARQAPGQG LEWMGIINPRGGSTSYAQKFQGRVTMTRDTSTSTVYMELSSLRSED TAVYYCARGKYWMYDWGKGTLVTVSS (SEQ ID NO: 101) |
| MEDI-24-VL | QSVLTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 102) |
| MEDI-25-VH | QVQLVQSGAEVKKPGASVKVSCKASGYAFTSYYMHWARQAPGQG LEWMGIINPRGGSASYAQKFQGRV SMTRDT STSTVYMELS SLRSED TAVYYCARGKYWMYDWGKGTLVTVSS (SEQ ID NO: 103) |
| MEDI-25-VL | QSVLTQPPSVSAAPGQKVTISCSGGSSNIGNSYVSWYQQLPGTAPKL LIYDNNKRPSGIPDRFSGSKSGTTATLAITGLQTGDEADYYCGTWVT STVWEWPFGTGTKLTVL (SEQ ID NO: 104) |
| MEDI-26-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-26-VL | QSVLTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 102) |
| MEDI-27-VH | QVQLVQSGAEVRKPGASVKVSCKASGYAFTSYYMHWARQAPGQG LEWMGIINPSGGSTSYAQKFQGRVTMTRDTSTSTVYMELSSLRPED TAVYYCARGKYWMYDWGKGTQVTVSS (SEQ ID NO: 105) |
| MEDI-27-VL | QSVLTQPPLVSAAPGQKVTISCSGGSSNIGNSYVSWYQRLPGTAPKL LIYDNNKRPSGIPDRFSGSKSGTSATLAITGLQTGDEADYYCGTWDT STVWEWPFGTGTKLTVL (SEQ ID NO: 106) |
| MEDI-28-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGNGTLVTVSS (SEQ ID NO: 107) |
| MEDI-28-VL | LPVLTQPPSVSAAPGQKVTISCSGGSSSIGNSYVSWYQQLPGAAPKL LIYDNNKRPSGIPDRFSGFRSGTSATLAITGLQTGDEADYYCGTWDT SPVWEWPFGTGTKLTVL (SEQ ID NO: 108) |
| MEDI-29-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTRVTVSS (SEQ ID NO: 109) |
| MEDI-29-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SPVWEWPFGTGTKLTVL (SEQ ID NO: 110) |
| MEDI-30-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-30-VL | QSVLTQPPSVSAAPGQKVTISCSGGSSNIGNSYVSWYQRLPGAAPKL LIYDNNKRPSGIPDRFSGSKSGTSATLAITGLQTGDEADYYCGTWDT STVWEWPFGTGTKLTVL (SEQ ID NO: 111) |
| MEDI-31-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-31-VL | QSVLTQPPSVSAAPGQKVTISCSGGSSSIGNSYVSWYQQLPGTAPKL LIYDNNKRPSGIPDRFSGSKSGTSATLAITGLQTGDEADYYCGTWAT SPVWEWPFGTGTKLTVL (SEQ ID NO: 112) |
| MEDI-32-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-32-VL | QSVLTQPPSVSAAPGQKVTISCSGGSSNIGNSYVSWYQQLPGTAPKL LIYDNNKRPSGIPDRFSGSKSGTSATLAITGLQTGDEADYFCGTWDT STAWEWPFGTGTKLTVL (SEQ ID NO: 113) |
| MEDI-33-VH | QVQLVQSGAEEKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 114) |
| MEDI-33-VL | QSALTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 115) |
| MEDI-34-VH | QVQLVQSGAEVKKPGASVKVSCKASGYAFTSYYMHWARQAPGQG LEWMGIINPSGGSTSYAQKFQGRVSMTRDTSTSTVYMELSSLRSEDT AVYYCARGKYWMYDWGKGTLVTVSS (SEQ ID NO: 116) |
| MEDI-34-VL | QSVLTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 102) |
| MEDI-35-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-35-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT SPVWEWPFGTGTKLTVL (SEQ ID NO: 110) |
| MEDI-36-VH | QVQLVQSGAEVKKPGASVKVSCKASGYAFTSYYMHWARQAPGQG LEWMGIINPSGGSASYAQKFQGRVTMTRDTSTSTVYMELSSLRSED TAVYYCARGKYWMYDWGKGTLVTVSS (SEQ ID NO: 96) |
| MEDI-36-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDS STVWEWPFGTGTKLTVL (SEQ ID NO: 117) |
| MEDI-37-VH | QVQLVQSGAEVKKPGASVKVSCKASGYAFTSYYMHWARQAPGQG LEWMGIINPRGGSTSYAQKFQGRVAMTRDTSTSTVYMELSSLRPED TAVYYCARGKYWMYDWGKGTLVTVSS (SEQ ID NO: 118) |
| MEDI-37-VL | QSVLTQPPSVSAAPGQKVTISCSGGGSSIGNSYVSWYQQLPGTAPKL LIYDNNKRPSGVPDRFSGSKSGTSATLAITGLQTGDEADYYCGTWD TSPVWEWPFGTGTKLTVL (SEQ ID NO: 119) |
| MEDI-38-VH | QVQLVQSGAEVKKPGASVKVSCKASGYAFTSYYMHWARQAPGQG LEWMGIINPSGGSASYAQKFQGRVTMTRDTSTSTVYMELSSLRSED TAVYYCARGKYWMYDWGKGTLVTVSS (SEQ ID NO: 96) |
| MEDI-38-VL | QSVLTQPPSVSAAPGQKVTISCSGGSSNIGNSYVSWYQQLPGTAPKL LIYDNNKRPSGIPDRFSGSKSGTSATLAITGLQTGDEADYFCGTWDT STVWEWPFGTGTKLTVL (SEQ ID NO: 102) |
| MEDI-39-VH | QVQLVQSGAEVKKPGASVKVSCKASGYAFTSYYMHWARQAPGQG LEWMGIINPRGGSTSYAQKFQGRVTMTRDTSTSTVYMELSSLRSED TAVYYCARGKYWMYDWGKGTLVTVSS (SEQ ID NO: 101) |
| MEDI-39-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDT STAWEWPFGTGTKLTVL (SEQ ID NO: 120) |
| MEDI-40-VH | QVQLVQSGAEVKKPGASVKVSCKASGYAFTSYYMHWARQAPGQG LEWMGIINPSGGSTSYAQKFQGRVTMTRDTSTSTVYMELSSLRSED TAVYYCARGKYWMYDWGKGTLVTVSS (SEQ ID NO: 82) |
| MEDI-40-VL | QSVLTQPPSVSAAPGQKVTISCSGGSSNIGNSYVSWYQQLPGTAPKL LIYDNNKRPSGIPDRFSGSKSGTSATLAITGLQTGDEADYYCGTWDS STVWEWPFGTGTKLTVL (SEQ ID NO: 117) |
| MEDI-41-VH | QVQLVQSGAEVRKPGASVKVSCKASGYAFTSYYMHWARQAPGQG LEWMGIINPSGGSTSYAQKFQGRVTMTRDTSTSTVYMELSSLRPED TAVYYCARGKYWMYDWGKGTLVTVSG (SEQ ID NO: 121) |
| MEDI-41-VL | QSVLTQPPSVSAAPGQKVTISCSGGSTNIGNSYVSWYQRLPGTAPKL LIYDNNKRPPGIPDRFSGSKSGTSATLAITGLQTGDEADYYCGTWDT STVWEWPFGTGTKLTVL (SEQ ID NO: 122) |
| MEDI-42-VH | QVQLVQSGAEVKKPGASVKVSCKASGYAFTSYYMHWARQAPGQG LEWVGIINPSGGSTSYAQKFQGRVTMTRDTSTSTVYMELSSLRSGDT AVYYCARGKYWMYDWGKGTLVTVSS (SEQ ID NO: 123) |
| MEDI-42-VL | QAVLTQPPSVSAAPGQKVTISCSGGSSNIGNSYVSWYQRLPGAAPKL LIYDNNKRPSGIPDRFSGSKSGTSATLAITGLQTGDEADYYCGTWDT STGWEWPFGTGTKLTVL (SEQ ID NO: 124) |
| MEDI-37GL-VH | QVQLVQSGAEVKKPGASVKVSCKASGYAFTSYYMHWVRQAPGQG LEWMGIINPRGGSTSYAQKFQGRVTMTRDTSTSTVYMELSSLRSED TAVYYCARGKYWMYDWGKGTLVTVSS (SEQ ID NO: 125) |
| MEDI-37GL-VL | QSVLTQPPSVSAAPGQKVTISCSGGGSSIGNSYVSWYQQLPGTAPKL LIYDNNKRPSGIPDRFSGSKSGTSATLGITGLQTGDEADYYCGTWDT SPVWEWPFGTGTKLTVL (SEQ ID NO: 126) |
| MEDI-37GL-HCDR1 | SYYMH (SEQ ID NO: 127) |
| MEDI-37GL-HCDR2 | IINPRGGSTSYAQKFQG (SEQ ID NO: 128) |
| MEDI-37GL-HCDR3 | GKYWMYD (SEQ ID NO: 129) |
| MEDI-37GL-LCDR1 | SGGGSSIGNSYVS (SEQ ID NO: 130) |
| MEDI-37GL-LCDR2 | DNNKRPS (SEQ ID NO: 131) |
| MEDI-37GL-LCDR3 | GTWDTSPVWEWP (SEQ ID NO: 132) |

In certain embodiments, an antibody or antigen-binding fragment thereof of the disclosure comprises a LCVR / HCVR sequence pair of AJOU-90-VL / AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| | |
|---|---|
| Sequence ID | Sequence |
| AJOU-1-VH | EVQLLESGGGLVQPGGSLRLSCAVSGFTFSNYAMSWVRQAPGKGL EWVSAISSGGGNIYYADSVKGRFTISRDNSKNTLYLQMNSLRAEDT AVYYCAKLRRYFDYWGQGTLVTVSS (SEQ ID NO: 133) |
| AJOU-2-VH | EVQLLESGGGLVQPGGSLRLSCAASGFTFSDYAMSWVRQAPGKGL EWVSAISSGGSSIYYADSVKGRFTISRDNSKNTLHLQMNSLRAEDT AVYYCARGPQRSATAVFDYWGQGTLVTVSS (SEQ ID NO: 134) |
| AJOU-3-VH | EVQLLESGGGLVQPGGSLRLSCAASGFTFSNYAMSWVRQAPGKGL EWVSWISPNSGNIYYADSVKGRFTISRDNSKNTLYLQMNSLRAEDT AVYYCARRPLSAAWSHSSYYNAMDVWGQGTLVTVSS (SEQ ID NO: 135) |
| AJOU-4-VH | EVQLLESGGGLVQPGGSLRLSCAASGFTFSGYAMSWVRQAPGKGL EWVSLISHSGSNTYYADSVKGRFTISRDNSKNTLYLQMNSLRAEDT AVYYCARPHRAFDYWGQGTLVTVSS (SEQ ID NO: 136) |
| AJOU-5-VH | EVQLLESGGGLVQPGGSLRLSCAASGFTFSNYAMSWVRQAPGKGL EWVSGISHGSGSIYYADSVKGRFTISRDNSKNTLYLQMNSLRAEDT AVYYCARPHRAFDYWGQGTLVTVSS (SEQ ID NO: 137) |
| AJOU-6-VH | EVQLLESGGGLVQPGGSLRLSCAASGFTFSNYAMSWVRQAPGKGL EWVSGISHGNGSIYYADSVKGRFTISRDNSKNTLYLQMNSLRAEDT AVYYCAKTGRHFDYWGQGTLVTVSS (SEQ ID NO: 138) |
| AJOU-7-VH | EVQLLESGGGLVQPGGSLRLSCAASGFTFSNYAMSWVRQAPGKGL EW VS SISP SGS SIYYAD S VKGRFTISRDNSKNTL YLQMNSLRAEDT A VYYCARSYRAFDYWGQGTLVTVSS (SEQ ID NO: 139) |
| AJOU-8-VH | EVQLLESGGGLVQPGGSLRLSCAASGFTFSNYAMSWVRQAPGKGL EWVSAISPSGGSIYYADSVKGRFTISRDNSKNTLYLQMNSLRAEDT AVYYCARAKRAFDYWGQGTLVTVSS (SEQ ID NO: 140) |
| AJOU-9-VH | EVQLLESGGGLVQPGGSLRLSCAASGFTFSNYAMSWVRQAPGKGL EWVSAISPGSGSTYYADSVKGRFTISRDNSKNTLYLQMNSLRAEDT AVYYCAKFRRHFDYWGQGTLVTVSS (SEQ ID NO: 141) |
| AJOU-10-VH | EVQLLESGGGLVQPGGSLRLSCAASGFTFSNYAMSWVRQAPGKGL EWVSAISSGGGNIYYADSVKGRFTISRDNSKNTLYLQMNSLRAEDT AVYYCARVHRAFDYWGQGTLVTVSS (SEQ ID NO: 142) |
| AJOU-69-VH | EVQLLESGGGLVQPGGSLRLSCAASGFTFSNYAMSWVRQAPGKGL EWVSAITSSGRSIYYADSVKGRFTISRDNSKNTLYLQMNSLRAEDT AVYYCARVHRAFDYWGQGTLVTVSS (SEQ ID NO: 143) |
| AJOU-70-VH | EVQLLESGGGLVQPGGSLRLSCAASGFTFSNYAMSWVRQAPGKGL EWVSAITSSGANIYYADSVKGRFTISRDNSKNTLYLQMNSLRAEDT AVYYCARVHRAFDYWGQGTLVTVSS (SEQ ID NO: 144) |
| AJOU-71-VH | EVQLLESGGGLVQPGGSLRLSCAASGFTFSNYAMSWVRQAPGKGL EWVSAITSSGGNIYYADSVKGRFTISRDNSKNTLYLQMNSLRAEDT AVYYCARVHRAFDYWGQGTLVTVSS (SEQ ID NO: 145) |
| AJOU-72-VH | EVQLLESGGGLVQPGGSLRLSCAASGFTFSNYAMSWVRQAPGKGL EWVSAITAGGGSIYYADSVKGRFTISRDNSKNTLYLQMNSLRAEDT AVYYCARVHRAFDYWGQGTLVTVSS (SEQ ID NO: 146) |
| AJOU-83-VH | EVQLLESGGGLVQPGGSLRLSCAASGFTFSRHAMAWVRQAPGKGL EWVSAITSSGRSIYYADSVKGRFTISRDNSKNTLYLQMNSLRAEDT AVYYCARVHRAFDYWGQGTLVTVSS (SEQ ID NO: 147) |
| AJOU-33-VL | QSVLTQPPSASGTPGQRVTISCSGSSSNIGNNYVNWYQQLPGTAPK LLIYDNSHRPSGVPDRFSGSKSGTSASLAISGLRSEDEADYYCGTW DASLSAYVFGGGTKLTVL (SEQ ID NO: 148) |
| AJOU-34-VL | QSVLTQPPSASGTPGQRVTISCSGSSSNIGNNNVSWYQQLPGTAPKL LIYANSKRPSGVPDRFSGSKSGTSASLAISGLRSEDEADYYCGSWD DSLSAYVFGGGTKLTVL (SEQ ID NO: 149) |
| AJOU-35-VL | QSVLTQPPSAPGTPGQRVTISCTGSSSNIGSNSVNWYQQLPGTAPKL LIYDDSHRPSGVPDRFSGSKSGTSASLAISGLRSEDEADYYCDAWD SSLSAYVFGGGTKLTVL (SEQ ID NO: 150) |
| AJOU-36-VL | QSVLTQPPSASGTPGQRVTLSCTGSSSNIGSNYVSWYQQLPGTAPK LLIYADSQRPSGVPDRFSGSKSGTSASLAISGLRSEDEADYYCGTW DDSLSGYVFGGGTKLTVL (SEQ ID NO: 151) |
| AJOU-37-VL | QSVLTQPPSASGTPGQRVTISCSSSSSNIGSNYVSWYQQLPGTAPKL LIYSDSHRPSGVPDRFSGSKSGTSASLAISGLRSEDEADYYCGSWDY SLSAYVFGGGTKLTVL (SEQ ID NO: 152) |
| AJOU-38-VL | QSVL TQPPSASGTPGQR VTISCTGSSSNIGNNTVSWYQQLPGT APKL LIYDNSHRPSGVPDRFSGSKSGTSASLAISGLQSEDEADYYCGSWD YSLSAYVFGGGTKLTVL (SEQ ID NO: 153) |
| AJOU-39-VL | QSVLTQPPSASGTPGQRVTISCTGSSSNIGNNDVNWYQQLPGTAPK LLIYYDSQRPSGVPDRFSGSKSGTSASLAISGLRSEDEADYYCATW DASLSAYVFGGGTKLTVL (SEQ ID NO: 154) |
| AJOU-40-VL | QSVLTQPPSASGTPGQRVTISCSGSSSNIGSNAVNWYQQLPGTAPKL LIYYDNQRPSGVPDRFSGSKSGTSASLAISGLRSEDEADYYCGTWD DSLNGYVFGGGTKLTVL (SEQ ID NO: 155) |
| AJOU-41-VL | QSVLTQPPSASGTPGQRVTISCSGSSSNIGNNAVTWYQQLPGTAPK LLIYDDSHRPSGVPDRFSGSKSGTSASLAISGLRSEDEADYYCGSW DYSLSAYVFGGGTKLTVL (SEQ ID NO: 156) |
| AJOU-42-VL | QSVLTQPPSASGTPGQRVTISCSGSSSNIGSNTFNWYQQLPGTAPKL LIYADSHRPSGVPDRFSGSKSGTSASLAISGLRSEDEADYYCGTWD YSLSGYVLGGGTKLTVL (SEQ ID NO: 157) |
| AJOU-77-VL | QSVLTQPPSASGTPGQRVTISCSGSSSNIGSNTFNWYQQLPGTAPKL LIYADSHRPSGVPDRFSGSKSGTSASLAISGLRSEDEADYYCGTWD YSLSGYVLGGGTKLTVL (SEQ ID NO: 157) |
| AJOU-78-VL | QSVLTQPPSASGTPGQRVTISCSGSSSNIGSNTFNWYQQLPGTAPKL LIYADSHRPSGVPDRFSGSKSGTSASLAISGLRSEDEADYYCGTWD YSLRGYVLGGGTKLTVL (SEQ ID NO: 158) |
| AJOU-79-VL | QSVLTQPPSASGTPGQRVTISCSGSSSNIGSNTFNWYQQLPGTAPKL LIYADSHRPSGVPDRFSGSKSGTSASLAISGLRSEDEADYYCGYWD YSLSGYVLGGGTKLTVL (SEQ ID NO: 159) |
| AJOU-80-VL | QSVLTQPPSASGTPGQRVTISCSGSSSNIGSNTFNWYQQLPGTAPKL LIYADSHRPSGVPDRFSGSKSGTSASLAISGLRSEDEADYYCGTWD YSLSGYVLGGGTKLTVL (SEQ ID NO: 157) |
| AJOU-86-VL | QSVLTQPPSASGTPGQRVTISCSGSSANSRTDGFNWYQQLPGTAPK LLIYADSHRPSGVPDRFSGSKSGTSASLAISGLRSEDEADYYCGTW DYSLSGYVLGGGTKLTVLG (SEQ ID NO: 160) |
| AJOU-87-VL | QSVLTQPPSASGTPGQRVTISCSGSAQFGSRDNFNWYQQLPGTAPK LLIYADSHRPSGVPDRFSGSKSGTSASLAISGLRSEDEADYYCGTW DYSLSGYVLGGGTKLTVLG (SEQ ID NO: 161) |
| AJOU-88-VL | QSVLTQPPSASGTPGQRVTISCSGSTKQMHNYQFNWYQQLPGTAP KLLIYADSHRPSGVPDRFSGSKSGTSASLAISGLRSEDEADYYCGT WDYSLSGYVLGGGTKLTVLG (SEQ ID NO: 162) |
| AJOU-89-VL | QSVLTQPPSASGTPGQRVTISCSGSLLRGENLQFNWYQQLPGTAPK LLIYADSHRPSGVPDRFSGSKSGTSASLAISGLRSEDEADYYCGTW DYSLSGYVLGGGTKLTVLG (SEQ ID NO: 163) |
| AJOU-90-VL | QSVLTQPPSASGTPGQRVTISCSGSPLFPDSGSFNWYQQLPGTAPKL LIYADSHRPSGVPDRFSGSKSGTSASLAISGLRSEDEADYYCGTWD YSLSGYVLGGGTKLTVLG (SEQ ID NO: 164) |
| AJOU-91-VL | QSVLTQPPSASGTPGQRVTISCSGSAALDLSPSFNWYQQLPGTAPKL LIYADSHRPSGVPDRFSGSKSGTSASLAISGLRSEDEADYYCGTWD YSLSGYVLGGGTKLTVLG (SEQ ID NO: 165) |
| AJOU-84-HCDR1 | RHAMA (SEQ ID NO: 166) |
| AJOU-85-HCDR2 | AITSSGRSIYYADSVKG (SEQ ID NO: 167) |
| AJOU-32-HCDR3 | VHRAFDY (SEQ ID NO: 168) |
| AJOU-96-LCDR1 | SGSPLFPDSGSFN (SEQ ID NO: 169) |
| AJOU-60-LCDR2 | ADSHRPS (SEQ ID NO: 170) |
| AJOU-68-LCDR3 | GTWDYSLSGYV (SEQ ID NO: 171) |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. 7,605,237 and U.S. 7,608,693, incorporated herein by reference in their entireties for all purposes.

TABLE 4

| | |
|---|---|
| Sequence ID | Sequence |
| REGN-VH-3 | QVQLVQSGAEVKKPGASVKVSCKASGYTFTNYGISWVRQAPGQG LEWMGWISVYNGKTNYAQKLQGRVTMTTDTSTTTAYMEMRSLR SDDTAVYYCARGSGYDLDYWGQGTLVSVSS (SEQ ID NO: 172) |
| REGN-VH-19 | EVQLVESGGGLVQPGGSLRLSCAASGFTFSSFWMTWVRQAPGKG LEWVANIKQDGSEKYYVDSVKGRFTISRDNAKNSLYLQMNSLRA EDTAVYYCARDPGRTMVRGGIRYYYGMDVWGQGTTVTVSS (SEQ ID NO: 173) |
| REGN-VH-35 | EVKLAESGGGLVQPGGSLRLSCAASGFTFSSHWMNWVRQAPGKG LEWVANIKQDGSDKYYVDSVKGRFTISRDNAKNSLYLQLNSLIAE DTAVYYCARDRGVRPPRGAFDIWGQGTMVTVSS (SEQ ID NO: 174) |
| REGN-VH-51 | QVQLVQSGAEVKKPGASVKVSCKASGYTFNSYGISWVRQAPGQG LEWMGWIRTYNGNTNYAQKLQGRVTMTTDTSTSTAYMELRSLR SDDTAVYYCARDEARIVVAGTTPYYYGMDVWGQGTTVTVSS (SEQ ID NO: 175) |
| REGN-VH-67 | QVQLVESGGGLVQPGGSLRLSCAVSGFTISDHYMSWIRQAPGKGL EWISYISSSGSKIYYADSVKGRFTISRDNAKNSLFLQMNSLRAEDT AVYYCARTRQLVGDYWGQGTLVTVSS (SEQ ID NO: 176) |
| REGN-VH-83 | EVQLVESGGGLVQPGRSLRLSCAASGFTFDNYAMHWVRQAPGK GLEWVSGIRWNSGSIGYADSVKGRFTISRDNAKNSLYLQMNSLRA EDTALYYCAKEGGYSGYRPGPFFDYWGQGTLVTVSS (SEQ ID NO: 177) |
| REGN-VH-99 | QVQLVQSGAEVKKPGASVKVSCKASGYTFTNYGISWVRQAPGQG LEWMGWISVYNGHTNYAQKLQGRVTMTTDTSTSTAYMELRSLR SDDTAVYYCARGSGYDFDSWGQGTLVTVSS (SEQ ID NO: 178) |
| REGN-VH-115 | QVQLVQSGAEVKKPGASVKVSCKASRYTFTSYDINWVRQATGQG LEWMGWMNPNSGNTGYAQKFQGRVTMTRNTSTSTAYMELSSLR SEDTAVYYCARVRRFFDYWGQGTLVTVSS (SEQ ID NO: 179) |
| REGN-VH-147 | QVQLVQSGPEVKKPGASVKVSCKASGYTFTNYGISWVRQAPGQG LEWMGWISVYNGNINYAQKLQGRVTMTTDTSTSTAYMDLRSLRS DDTAVYYCARGSGYDFDYWGQGTLVTVSS (SEQ ID NO: 180) |
| REGN-VH-163 | QVQLVQSGAEVKKPGASVKVSCKDSAYTFNRYGISWVRQAPGQG LEWMGWISAYTGNTVYAQKLQGRVTMTTDNSTSTAYMELRSLR SDDTAVYYCARDKSIFGVVRGFDYWGQGTLVTVSS (SEQ ID NO: 181) |
| REGN-VL-11 | AIQMTQSPSSLSASVGDRVTITCRASQGIRNALGWYQQKPGKAPK LLIYAASSLQSGVPSRFSGSGSGTDFTLTFSSLQPEDFATYYCLQDF NYPYTFGQGTKLEIK (SEQ ID NO: 182) |
| REGN-VL-27 | DIQMTQSPSSVSASVGDRVTISCRASQGVSSWLAWYQQKPGNAP KLLISAASSIQSGVPSRFSGSGSGTDFTLTISSLQPEDFATYYCQQA NSFPLTFGGGTKVEIK (SEQ ID NO: 183) |
| REGN-VL-43 | DIQMTQSPSSVSASVGDRVTITCRASQGISSWLAWYQQKPGKAPK LLIYAASSFQSGVPSRFSGSGSGTDFTLTISSLQPEDFATYFCQQAN SFPLTFGGGTTVEIK (SEQ ID NO: 184) |
| REGN-VL-59 | DIQMTQSPSSVSASVGDRVTITCRASQDISIWLAWYQQSPGKAPKL LINVASRLQSGVPSRFSGSGSGTDFTLTINSLQPEDFVTYYCQQAN SFPITFGQGTRLATK (SEQ ID NO: 185) |
| REGN-VL-75 | DIQLTQSPSFLSASVGDRVTITCWASQGISSYLAWYQQKPGKAPKL LIFAASTLQSGVPSRFSGSGSGTEFTLTISSLQPEDFATYYCQQLNS YPLTFGGGTKVEIR (SEQ ID NO: 186) |
| REGN-VL-91 | EIVMTQSPATLSVSPGERATLSCRASQSVNYNLAWYQHKPGQAPR LLIYGASTRATGIPARFSGSGSGTEFTLTISSLQSEDFAVYYCQQYN NWPLTFGGGTKVEIK (SEQ ID NO: 187) |
| REGN-VL-107 | AIQMTQSSSSLSASVGDRVTITCRASQAIRNALGWYQQKPGKAPK VLIYAASSLQSGIPSRFSGSGSGTDFTLTISSLQPEDFATYYCLQDY DYPYTFGQGTKLEIK (SEQ ID NO: 188) |
| REGN-VL-123 | DIQLTQSPSFLSASVGDRVTITCWASQGIISYLAWYQQKPGKAPKL LIYAASTLHSGVPSRFSGSGSGTEFTLTISSLQPEDFATYYCHQLKS YPITFGQGTRLEIK (SEQ ID NO: 189) |
| REGN-VL-155 | AIQMTQSPSSLSASVGDRVTITCRASQDIRNALGWYQQKPGKAPK LLIYAASSLQSGVPSRFSGSASGTDFTLTISSLQPEDFAAYYCLQDY NYPYTFGQGTKLEIK (SEQ ID NO: 190) |
| REGN-VL-171 | EIVMTQSPVTLSLSPGERATLPCRASQSVSSSLAWYQQKAGQSPRL LIYGASTRATGIPARFSGSGSGTEFTLTISNLQSEDFAVYYCQQYN NWPLTFGGGTKVEIK (SEQ ID NO: 191) |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington’s Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat CSU in a patient. Aerosolized antibodies can be prepared as described in, for example, US 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition’s target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. 8,945,559.

### Dosage

The amount of IL-4R antagonist (e.g., anti-IL-4R antibody) administered to a subject according to the methods described herein is, generally, a therapeutically effective amount. As used herein, the phrase “therapeutically effective amount” means an amount of IL-4R antagonist that results in improvement in one or more CSU-associated PRO measures (as defined elsewhere herein). A “therapeutically effective amount” also includes an amount of IL-4R antagonist that inhibits, prevents, lessens, or delays the progression of CSU in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, 300 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1x, about 1.2x, about 1.3x, about 1.4x, about 1.5x, about 1.6x, about 1.7x, about 1.8x, about 1.9x, about 2.0x, about 2.5x, about 3.0x, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg. In certain embodiments, the methods comprise an initial dose or loading dose of about 400 mg or about 600 mg of an IL-4R antagonist. In certain embodiments, the methods comprise one or more secondary doses or maintenance doses of about 200 mg or about 300 mg of the IL-4R antagonist.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of more than 30 kg, and the IL-4R antagonist is administered at an initial dose of about 400 mg and one or more secondary doses of about 400 mg, and the secondary doses are administered every other week (q2w). In certain exemplary embodiments, a subject is a pediatric subject having a body weight of more than 30 kg, and the IL-4R antagonist is administered at an initial dose of about 300 mg and one or more secondary doses of about 300 mg, and the secondary doses are administered every other week (q2w). In certain exemplary embodiments, a subject is a pediatric subject having a body weight of more than 30 kg, and the IL-4R antagonist is administered at an initial dose of about 200 mg and one or more secondary doses of about 400 mg, and the secondary doses are administered every other week (q2w). In particularly exemplary embodiments, a subject is a pediatric subject having a body weight of more than 30 kg, and the IL-4R antagonist is administered at an initial dose or loading dose of about 400 mg and one or more secondary doses or maintenance doses of about 200 mg, and the secondary doses are administered every other week (q2w).

In certain exemplary embodiments, a subject is a pediatric subject having a body weight of 30 kg or less and a body weight of at least 15 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of 30 kg or less and a body weight of at least 15 kg, and the IL-4R antagonist is administered at an initial dose of about 600 mg and one or more secondary doses of about 600 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 500 mg and one or more secondary doses of about 500 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 400 mg and one or more secondary doses of about 400 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 300 mg and one or more secondary doses of about 300 mg, and the secondary doses are administered every four weeks (q4w). In particularly exemplary embodiments, a subject is a pediatric subject having a body weight of 30 kg or less and a body weight of at least 15 kg, and the IL-4R antagonist is administered at an initial dose of about 600 mg and one or more secondary doses or maintenance doses of about 300 mg, and the secondary doses are administered every four weeks (q4w).

In certain exemplary embodiments, a subject is a pediatric subject having a body weight of less than 15 kg and at least 5 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of less than 15 kg and at least 5 kg, and the IL-4R antagonist is administered at an initial dose of about 300 mg and one or more secondary doses of about 300 mg, and the secondary doses are administered every four weeks (q4w). In certain exemplary embodiments, a subject is a pediatric subject having a body weight of less than 15 kg and at least 5 kg, and the IL-4R antagonist is administered at an initial dose of about 250 mg and one or more secondary doses of about 250 mg, and the secondary doses are administered every four weeks (q4w). In particularly exemplary embodiments, a subject is a pediatric subj ect having a body weight of less than 15 kg and at least 5 kg, and the IL-4R antagonist is administered at an initial dose of about 200 mg and one or more secondary doses of about 200 mg, and the secondary doses are administered every four weeks (q4w).

In certain exemplary embodiments, a subject is an adolescent subject having a body weight of less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some exemplary embodiments, a subject is an adolescent subject having a body weight of less than 60 kg, and the IL-4R antagonist is administered at an initial dose of about 400 mg and one or more secondary doses or maintenance doses of about 200 mg, and the secondary doses are administered every other week (q2w). In certain exemplary embodiments, a subject is an adolescent subject having a body weight that is greater than or equal to 30 kg and less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some exemplary embodiments, a subject is an adolescent subject having a body weight that is greater than or equal to 30 kg and less than 60 kg, and the IL-4R antagonist is administered at an initial dose of about 400 mg and one or more secondary doses or maintenance doses of about 200 mg, and the secondary doses are administered every other week (q2w).

In certain exemplary embodiments, a subject is an adolescent subject having a body weight of at least 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In particularly exemplary embodiments, a subject is an adolescent subject having a body weight of at least 60 kg, and the IL-4R antagonist is administered at an initial dose of about 600 mg and one or more secondary doses or maintenance doses of about 300 mg, and the secondary doses are administered every other week (q2w).

In certain exemplary embodiments, a subject is an adult, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In particularly exemplary embodiments, a subject is an adult, and the IL-4R antagonist is administered at an initial dose of about 600 mg and one or more secondary doses or maintenance doses of about 300 mg, and the secondary doses are administered every other week (q2w).

In certain exemplary embodiments, an IL-4R antagonist is administered at a concentration of 150 mg/mL using a prefilled device. In some embodiments, a 150 mg/mL IL-4R antagonist solution in a pre-filled device is used to deliver 300 mg IL-4R antagonist in a 2 mL injection. In certain exemplary embodiments, an IL-4R antagonist is administered at a concentration of 175 mg/mL using a prefilled device. In some embodiments, a 175 mg/mL IL-4R antagonist solution in a pre-filled device is used to deliver 200 mg IL-4R antagonist in a 1.14 mL injection.

### Combination Therapies

Certain embodiments of the methods described herein comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat CSU or an associated condition or complication comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

In exemplary embodiments, an additional therapeutic agent administered in combination with the IL-4R antagonist is a background therapy. In exemplary embodiments, a background therapy includes one or both of an antihistamine and an anti-IgE antibody. In certain embodiments, the method leads to reduced need of the background therapy. For example, in certain embodiments, the method leads to reduced dose and/or reduced frequency of the background therapy.

The additional therapeutic agent may be, e.g., another IL-4R antagonist (e.g., one or more suitable IL-4R antagonists listed in Tables 1-4), an IgE antagonist, an antihistamine, an IL-1 antagonist (including, e.g., an IL-1 antagonist as set forth in US Pat. No. 6,927,044), an IL-5 antagonist, an IL-5R antagonist, an IL-6 antagonist, an IL-6R antagonist (including, e.g., an anti-IL-6R antibody as set forth in US Pat. No. 7,582,298), or an IL-17 antagonist.

In an exemplary embodiment, the additional therapeutic is an H1 antihistamine. In some embodiments, the H1 antihistamine is selected from the group consisting of cetirizine, levocetirizine, fexofenadine, loratadine, desloratadine, bilastine, and rupatadine.

In a further exemplary embodiment, the additional therapeutic is an anti-IgE antibody. In some embodiments, the anti-IgE antibody is omalizumab. In some embodiments, the anti-IgE antibody is ligelizumab.

In some embodiments, an additional therapeutic agent administered in combination with the IL-4R antagonist is a vaccine. In certain exemplary embodiments, the vaccine is a viral vaccine or a bacterial vaccine. In certain exemplary embodiments, the vaccine is a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB,PedvaxHIB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PPSV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RV1 (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html). In some embodiments, the vaccine is for tetanus, diphtheria, pertussis and/or seasonal trivalent/quadrivalent influenza vaccine.

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine is concurrently treated for CSU with an IL-4R antagonist.

In certain embodiments, treatment with an IL-4R antagonist is suspended or terminated prior to treatment with the vaccine. In certain embodiments, treatment with the IL-4R antagonist is suspended about 1 to about 9 (e.g., about 1, about 1½, about 2, about 2½, about 3, about 3½, about 4, about 4½, about 5, about 5½, about 6, about 6½, about 7, about 7½, about 8, about 8½, about 9, or more) weeks prior to administration of the vaccine. In some embodiments, treatment with the IL-4R antagonist is suspended about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, treatment with the IL-4R antagonist is resumed subsequent to treatment with the vaccine. In certain embodiments, treatment with the IL-4R antagonist is resumed about 1 to about 14 (e.g., about 1, about 1½, about 2, about 2½, about 3, about 3½, about 4, about 4½, about 5, about 5½, about 6, about 6½, about 7, about 7½, about 8, about 8½, about 9, about 9½, about 10, about 10½, about 11, about 11½, about 12, about 12½, about 13, about 13½, about 14, about 14½, or more) weeks subsequent to administration of the vaccine. In some embodiments, treatment with the IL-4R antagonist is resumed about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

In certain embodiments, the effectiveness of the IL-4R antagonist is not decreased by administration in combination with the vaccine, or by subsequent administration of the vaccine.

In some embodiments, the effectiveness of the vaccine is not decreased by administration in combination with the IL-4R antagonist, or by previous and/or subsequent administration of the IL-4R antagonist. In some embodiments, the subject develops seroprotective neutralization titers to the vaccine when the vaccine is co-administered with the IL-4R antagonist.

In certain exemplary embodiments, a subject is administered a vaccine described herein, wherein before, during, or after administration of the vaccine, the subject is administered at least one dose of IL-4R antagonist.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w),once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n x 7 days) ±3 days, e.g., (n x 7 days) ±2 days, (n x 7 days) ±1 day, or (n x 7 days), wherein “n” designates the number of weeks, e.g., 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist, or may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 600 mg or 400 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 300 mg or 200 mg of IL-4R antagonist may be administered followed by secondary doses/maintenance doses of about 300 mg or about 200 mg, respectively. In one embodiment, a loading dose may be split, e.g., two or more doses administered at different time points, e.g., two loading doses wherein a second loading dose is administered two weeks after a first loading dose.

In certain embodiments, the initial dose is about 50 mg to about 600 mg of the IL-4R antagonist. In one embodiment, the initial dose is 600 mg of the IL-4R antagonist. In another embodiment, the initial dose is 400 mg of the IL-4R antagonist.

In certain embodiments, the secondary dose(s) are about 50 mg to about 600 mg of the IL-4R antagonist. In one embodiment, the maintenance dose is 300 mg of the IL-4R antagonist. In one embodiment, the maintenance dose is 200 mg of the IL-4R antagonist.

In certain embodiments, an initial dose is three times a maintenance dose. In certain embodiments, an initial dose is two times a maintenance dose. In certain embodiments, an initial dose is equal to a maintenance dose.

In some embodiments, the subject is a child and has a body weight of less than 15 kg and at least 5 kg, the initial dose comprises 200 mg of the antibody or antigen-binding fragment thereof, and the one or more secondary doses comprises 200 mg of the antibody or antigen-binding fragment thereof administered every four weeks (q4w).

In some embodiments, the subject is a child and has a body weight of 30 kg or less and at least 15 kg, the initial dose comprises 600 mg of the antibody or antigen-binding fragment thereof, and the one or more secondary doses comprises 300 mg of the antibody or antigen-binding fragment thereof administered every four weeks (q4w).

In some embodiments, the subject is a child and has a body weight of 30 kg or less and at least 15 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject is a child and has a body weight of greater than 30 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In exemplary embodiments, the subject is an adolescent and has a body weight that is greater than or equal to 30 kg and less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of more than 60 kg, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In some embodiments, the subject is an adult, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-4R antagonist and a second therapeutic agent, to a patient to treat CSU or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 600 mg of the IL-4R antagonist may be administered after which one or more doses (e.g., 2, 3, 4, 5, 6, 7, 8, or more) of a second therapeutic agent (e.g., an H1 antihistamine or an anti-IgE antibody, as described elsewhere herein) may be administered to treat, alleviate, reduce or ameliorate one or more symptoms of CSU. In some embodiments, the IL-4R antagonist is administered at one or more doses (e.g., 2, 3, 4, 5, 6, 7, 8, or more) resulting in an improvement in one or more CSU-associated parameters followed by the administration of a second therapeutic agent to prevent recurrence of at least one symptom of CSU. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, the IL-4R antagonist is administered for at least 24 weeks.

In certain embodiments, a kit comprising a dosage form of an antibody, or an antigen-binding fragment thereof, that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, for the treatment of CSU is provided. In certain embodiments, the antibody or antigen-binding fragment thereof comprises a heavy chain variable region (HCVR) sequence of SEQ ID NO: 1 and a light chain variable region (LCVR) sequence of SEQ ID NO: 2. In certain embodiments, the antibody is dupilumab.

The kit can comprise a label or package insert, wherein the label or package insert comprises instructions for administering the dosage form for the treatment of CSU. The instructions can recite a dosing regimen described further herein for the treatment of CSU.

### Treatment Populations

The methods provided herein include administering to a subject in need thereof a therapeutic composition comprising an IL-4R antagonist. The expression “a subject in need thereof” means a human or non-human animal that exhibits one or more symptoms or indicia of CSU, or who has been diagnosed with CSU.

In a related embodiment, a “subject in need thereof” may be a subject who, prior to receiving an IL-4R antagonist, has been prescribed or is currently taking an antihistamine. In some embodiments, the subject is currently taking an H1 antihistamine. In exemplary embodiments a subject is currently taking an H1 antihistamine selected from the group consisting of cetirizine, levocetirizine, fexofenadine, loratadine, desloratadine, bilastine, and rupatadine. For example, methods that comprise administering an IL-4R antagonist to a patient who has been taking a regular course an H1 antihistamine for six or more weeks immediately preceding the administration of the IL-4R antagonist (such prior treatments are referred to herein as “background treatments”) are provided.

In other exemplary embodiments, a “subject in need thereof” may be a subject who, prior to receiving an IL-4R antagonist, has been prescribed or is currently taking an IgE antagonist. Therapeutic methods in which background treatments are continued in combination with administration of the IL-4R antagonist are provided. For example, methods that comprise administering an IL-4R antagonist to a patient who has been taking a regular course of an IgE antagonist immediately preceding the administration of the IL-4R antagonist (such prior treatments are referred to herein as “background treatments”) are provided.

In yet other embodiments, the amount of the H1 antihistamine, the IgE antagonist, or both, is gradually decreased prior to or after the start of IL-4R antagonist administration.

In another exemplary embodiment, a “subject in need thereof” has a diagnosis of CSU refractory to H1 antihistamines prior to receiving the IL-4R antagonist. In some embodiments, the CSU symptoms of the subject persist despite treatment with an H1 antihistamine (i.e., the subject is resistant to treatment with an H1 antihistamine.)

In a further exemplary embodiment, a “subject in need thereof” is naive to IgE antagonists such as omalizumab (i.e., the subject has not been previously treated with an IgE antagonist). In another embodiment, a “subject in need thereof” is intolerant to IgE antagonists such as omalizumab (i.e., the subject experiences adverse effects associated with IgE antagonist treatment.) In another embodiment, a “subject in need thereof” is an incomplete responder to IgE antagonists including omalizumab (i.e., the subject continues to experience CSU symptoms despite treatment with the IgE antagonist.)

In some embodiments, a “subject in need thereof” is selected from the group consisting of: a subject age 18 years old or older, a subject 12 years or older, a subject age 12 to 17 years old (12 to < 18 years old), a subject age 6 to 11 years old (6 to <12 years old), a subject aged 2 to 11 years old (2 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months. In particularly exemplary embodiments, a subject is a child 6 to <12 years old (also referred to herein as a “pediatric” subject). In certain embodiments, a subject in need thereof is a child 2 to <6 years old having a body weight of at least 5 kg and less than 15 kg. In certain embodiments, a subject in need thereof is a child 6 to <12 years old having a body weight of more than 30 kg. In certain embodiments, a subject in need thereof is a child 6 to <12 years old having a body weight of 30 kg or less and at least 15 kg. In certain embodiments, a subject in need thereof is an adolescent 12 to <18 years old having a body weight of at least 60 kg. In exemplary embodiments, a subject in need thereof is an adolescent 12 to <18 years old having a body weight of less than 60 kg. In other exemplary embodiments, a subject in need thereof is an adolescent 12 to <18 years old having a body weight that is greater or equal to 30 kg and less than 60 kg.

In certain embodiments, methods for treating CSU are provided comprising: (a) selecting a subject that exhibits a blood eosinophil level of at least 300 cells per microliter; and (b) administering to the subject a pharmaceutical composition comprising an IL-4R antagonist.

In certain embodiments, methods for treating CSU are provided comprising: (a) selecting a patient that exhibits a blood eosinophil level of 200-299 cells per microliter; and (b) administering to the patient a pharmaceutical composition comprising an IL-4R antagonist.

In certain embodiments, methods for treating CSU are provided comprising: (a) selecting a patient that exhibits a blood eosinophil level of less than 200 cells per microliter; and (b) administering to the patient a pharmaceutical composition comprising an IL-4R antagonist.

In certain embodiments, methods for treating CSU are provided comprising: (a) selecting a patient that exhibits a blood eosinophil level of at least 150 cells per microliter; and (b) administering to the patient a pharmaceutical composition comprising an IL-4R antagonist.

In certain embodiments, methods for treating CSU are provided comprising: (a) selecting a patient that exhibits a blood eosinophil level of at least 100 cells per microliter; and (b) administering to the patient a pharmaceutical composition comprising an IL-4R antagonist.

In certain embodiments, methods for treating CSU are provided comprising: (a) selecting a patient that exhibits a blood eosinophil level of less than 100 cells per microliter; and (b) administering to the patient a pharmaceutical composition comprising an IL-4R antagonist.

In some embodiments, a “subject in need thereof” is a subject who is treated with a vaccine, e.g., viral vaccine or a bacterial vaccine. In some embodiments, the vaccine is a live vaccine, e.g., a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHIB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PPSV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RV1 (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html).

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine concurrently is treated for CSU with an IL-4R antagonist. In some embodiments, the subject suspends treatment with an IL-4R antagonist prior to administration of the vaccine.

In certain embodiments the subject suspends treatment with the IL-4R antagonist about 1 to about 9 (e.g., about 1, about 1½, about 2, about 2½, about 3, about 3½, about 4, about 4½, about 5, about 5½, about 6, about 6½, about 7, about 7½, about 8, about 8½, about 9, or more) weeks prior to administration of the vaccine. In certain embodiments, the subject suspends treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, the subject resumes treatment with the IL-4R antagonist subsequent to treatment with the vaccine. In certain embodiments, the subject resumes treatment with the IL-4R antagonist 1 to 14 (e.g., about 1, about 1½, about 2, about 2½, about 3, about 3½, about 4, about 4½, about 5, about 5½, about 6, about 6½, about 7, about 7½, about 8, about 8½, about 9, about 9½, about 10, about 10½, about 11, about 11½, about 12, about 12½, about 13, about 13½, about 14, about 14½, or more) weeks subsequent to administration of the vaccine. In certain embodiments, the subject resumes treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

### Methods for Assessing Pharmacodynamic CSU-Associated Parameters

Methods for assessing one or more pharmacodynamic CSU-associated parameters in a subject in need thereof, caused by administration of a pharmaceutical composition comprising an IL-4R antagonist, are provided. A reduction in the incidence of CSU symptoms or an improvement in a CSU-associated PRO measure may correlate with an improvement in one or more pharmacodynamic CSU-associated parameters; however, such a correlation is not necessarily observed in all cases.

Examples of “pharmacodynamic CSU-associated parameters” include, for example, the following: (a) biomarker expression levels and (b) serum protein and RNA analysis. An “improvement in a pharmacodynamic CSU-associated parameter” means, for example, a decrease from baseline of one or more biomarkers, such as IgE, eosinophil level, c-reactive protein (CRP), IL-6, D-dimer, medium platelet volume (MPV), IL-17, IL-18, IL-31, IL-33, and metalloproteinase-9. As used herein, the term “baseline,” with regard to a pharmacodynamic CSU-associated parameter, means the numerical value of the pharmacodynamic CSU-associated parameter for a patient prior to or at the time of administration of a pharmaceutical composition described herein.

To assess a pharmacodynamic CSU-associated parameter, the parameter is quantified at baseline and at a time point after administration of the pharmaceutical composition. For example, a pharmacodynamic CSU-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic CSU-associated parameter (e.g., an increase or decrease, as the case may be, depending on the specific parameter being measured).

In certain embodiments, administration of an IL-4R antagonist to a patient causes a change, such as a decrease or increase, in expression of a particular biomarker. CSU-associated biomarkers include, but are not limited to total IgE, c-reactive protein (CRP), IL-6, D-dimer, medium platelet volume (MPV), IL-17, IL-18, IL-31, IL-33, and metalloproteinase-9. For example, administration of an IL-4R antagonist to a CSU patient can cause a decrease in total serum IgE levels. The decrease can be detected at about week 1, about week 2, about week 3, about week 4, about week 5, or longer following administration of the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist or IL-4/IL-13 signaling (e.g., by measuring soluble IL,-4Rα, IL-4, IL-13, etc.). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in US 8,945,559, which is incorporated herein by reference in its entirety for all purposes.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D.N. Glover ed. 1985); Oligonucleotide Synthesis (M.J. Gait ed. 1984); Nucleic Acid Hybridization [B.D. Hames & S.J. Higgins eds. (1985)]; Transcription And Translation [B.D. Hames & S.J. Higgins, eds. (1984)]; Animal Cell Culture [R.I. Freshney, ed. (1986)]; Immobilized Cells And Enzymes [IRL Press, (1986)]; B. Perbal, A Practical Guide To Molecular Cloning (1984); F.M. Ausubel et al. (eds.), Current Protocols in Molecular Biology, John Wiley & Sons, Inc. (1994).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions featured in the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Examples is the human anti-IL-4R antibody named dupilumab (also referred to herein as “mAb1” or DUPIXENT®).

### Example 1. Three Randomized, Double-Blind, Placebo-Controlled, Multi-Center, Parallel-Group Studies of Dupilumab in Patients With Chronic Spontaneous Urticaria (CSU) Who Remain Symptomatic Despite the Use of H1 Antihistamine Treatment in Patients Naïve to Omalizumab and in Patients Who are Intolerant or Incomplete Responders to Omalizumab

### Study Rationale

Chronic spontaneous urticaria (CSU), also referred to as chronic idiopathic urticaria (CIU), is a common condition characterized by the spontaneous appearance of itchy wheals (hives) with or without angioedema persisting for more than 6 weeks without a specific known cause. Chronic spontaneous urticaria patients with and without angioedema experience debilitating hives and pruritus secondary to mast cell and basophil dysregulation. Degranulation of these cell types by Fc gamma receptor (FcεRI) activation, through agonistic autoantibodies or cell surface-bound immunoglobulin E (IgE) cross-linked by antigen, release histamine and other pro-inflammatory mediators leading to local tissue edema and pruritus. Many symptoms of urticaria are mediated primarily by the actions of histamine (a mast cell mediator) on the H1-receptors, and treatment with H1-antihistamines (H1—AH) is a mainstay of therapy (See Zuberbier T, et al. The EAACI/GA2LEN/EDF/WAO guideline for the definition, classification, diagnosis and management of urticaria. Allergy. 2018;73(7): 1393-414.) Approximately 50% of patients achieve symptomatic control with conventional H1-AH therapy (See Kaplan AP. Chronic spontaneous urticaria: pathogenesis and treatment considerations. Allergy Asthma Immunol Res. 2017;9(6):477-82.) Even with up-titration of antihistamines, approximately 40% to 50% of patients remain symptomatic.

The mechanism by which omalizumab exerts its therapeutic effects is likely constrained to reduction in serum IgE and consequent down-regulation of IgE receptors. Targeting IgE by omalizumab has been successful in treating CSU patients but not all patients are equally responsive to this therapy (See Maurer M, et al. Omalizumab for the treatment of chronic idiopathic or spontaneous urticaria. N Engl J Med. 2013;368(10):924-35.) Therefore, there remains an unmet need.

One possible way to meet this need is through novel therapies that target signaling pathways important for mast cell and basophil survival and function. Blockade of IL-4/IL-13 by dupilumab represents a novel therapeutic approach for CSU patients. As this is a novel therapy that acts further upstream than IgE-targeted therapies, the clinical trials described here show efficacy of dupilumab in patients who have failed antihistamines alone, or who have failed both antihistamines and omalizumab or who were intolerant to omalizumab. Each of the 2 studies is equally important to begin to address to what extent dupilumab inhibits hives and/or angioedema through IgE-dependent and independent mechanisms.

Dupilumab is a fully human monoclonal antibody (mAb) directed against the interleukin-4 receptor alpha subunit (IL-4Rα), which is a component of interleukin (IL)-4 receptors Type I and Type II, the latter being also a receptor for IL-13. The binding of dupilumab to IL-4Rα results in blockade of both IL-4 and IL-13 signaling.

### Study Overview

The protocol included 3 studies in CSU patients who remain symptomatic despite the use of H1-AH treatment - 1 study includes patients who are omalizumab naive (Study A) and the other study includes patients who are intolerant or incomplete responders to omalizumab (Study B). Study C will be conducted in the same study population with a similar design as Study A to meet Health Authority requirements to provide data from two adequate and well-controlled clinical trials. The 3 studies are of similar design, 2 studies in participants who are omalizumab naive (Study A and Study C) and 1 study in participants who are intolerant or incomplete responders to omalizumab (Study B). Study A and Study C include adults, adolescents (≥12 to <18 years) and children (≥6 to <12 years in some selected countries). Study B includes adults and adolescents. The selected dosing regimen is dupilumab 300 mg every 2 weeks (q2w) with a loading dose of 600 mg for adults; 300 mg q2w with a loading dose of 600 mg for adolescents >60 kg at screening OR 200 mg q2w with a loading dose of 400 mg for adolescents <60 kg at screening; and 200 mg q2w with a loading dose of 400 mg for children ≥6 to<12 years of age with weight ≥30 kg at screening OR 300 mg q4w with a loading dose of 600 mg for children ≥6 to <12 years of age with weight <30 kg and ≥15 kg at screening.

In all three studies, the target population consists of CSU patients who remain symptomatic despite treatment with H1-AH alone as these patients have a significant unmet medical need. The updated international guideline on the definition, classification, diagnosis and management of urticaria (Zuberbier T, et al. The EAACI/GA2LEN/EDF/WAO guideline for the definition, classification, diagnosis and management of urticaria. Allergy, 2018;73(7): 1393-414.) provides evidence-based recommendations and a treatment algorithm. Steps 1 and 2 of this algorithm is the use of non-sedating H1-AHs at approved, or increased doses (up to 4-fold), respectively. Step 3 treatment options are omalizumab, ciclosporin A, or montelukast (LTRA). This protocol allows the use of H1-AH at up to 4-fold the approved doses as background medication (Steps 1 and 2).

The total anticipated number of participants across the 3 studies is approximately 384 randomized participants.

For Study A, conducted in the omalizumab naïve population, approximately 130 participants will be randomized. This corresponds to approximately 65 participants who are randomly assigned to each intervention arm. Approximately 5% of participants enrolled will be adolescents and up to approximately 5% of participants enrolled will be children ≥6 to <12 years of age (both children and adolescents recruited in some selected countries). The actual number of participants randomized in Study A was 138.

For Study B, conducted in the omalizumab intolerant or incomplete responder population, approximately 104 participants were to be randomized. Approximately 5% of participants enrolled would be adolescents (recruited in some selected countries). The study recruitment ended, and the final number of participants randomized in Study B was 108. An interim analysis was performed when the first 83 randomized participants had completed their 24-week treatment period, by the interim analysis cut-off date, and met futility criteria. The study treatment is being stopped for the participants still on study treatment and all participants should complete their follow up period.

Omalizumab incomplete responders are defined as participants treated with at least 300 mg omalizumab every 4 weeks (q4w) for at least 3 months (minimum of 3 injections) and who have had an inadequate response resulting in omalizumab discontinuation, as confirmed by Investigator assessment.

### Objectives

Primary Objective: To demonstrate the efficacy of dupilumab in study participants with CSU who remain symptomatic despite the use of H1-AH (Study A: omalizumab naive; Study B: omalizumab intolerant or incomplete responders).

Secondary Objectives:
To demonstrate the efficacy of dupilumab on urticaria activity composite endpoint and itch or hives, separately, at various time points.
To demonstrate the efficacy of dupilumab on angioedema.
To demonstrate the efficacy of dupilumab on urticaria control.
To demonstrate improvement in health-related quality-of-life and overall disease status and severity.
To evaluate the ability of dupilumab in reducing the proportion of patients who require treatment with oral corticosteroids (OCS).
To evaluate safety outcome measures.
To evaluate immunogenicity of dupilumab.

Other Objectives:
To demonstrate outcome measures in the urticaria composite score and or its components
To demonstrate health-related quality-of-life and health status measures

### Endpoints

Primary Endpoints:
Change from baseline in weekly itch severity score (ISS7) at Week 24 (except EU and EU reference countries).
For EU and EU reference countries only: Change from baseline in weekly urticaria activity score (UAS7, composite patient reported itch and hive score) at Week 24.

Secondary Endpoints:
Change from baseline in weekly urticaria activity score (UAS7) at Week 12* and Week 24 (except EU and EU reference countries).
Change from baseline in ISS7 at Week 12* and at Week 24 (in EU and EU reference countries).
Change from baseline in weekly hives severity score (HSS7) at Week 12 and Week 24.
Time to ISS7 minimally important (MID) (ISS7 ≥5) response.
Proportion of ISS7 MID (≥5 points) responders at Week 12* and Week 24*.
Change from baseline in ISS7 at all time points (onset of action is assessed by the first p<0.05 that remains significant at subsequent measures until Week 24).
Proportion of patients with UAS7≤6 at Week 12* and Week 24*.
Proportion of patients with UAS7 = 0 at Week 12* and Week 24*.
Change from baseline in angioedema activity score over 7 days (AAS7) at Week 12 and Week 24.
Change from baseline in urticaria control test (UCT) at Week 12 and Week 24.
Proportion of well-controlled patients (UCT ≥12) at Week 12 and Week 24.
Change from baseline in health-related quality-of-life (HRQoL) as measured by Dermatology Life Quality Index (DLQI) in patients ≥16 years old, and in Children’s Dermatology Life Quality Index (CDLQI) in patients ≥6 to <16 years old at Week 12 and Week 24.
Patient Global Impression of Change (PGIC) of CSU at Week 12 and Week 24.
Change from baseline in Patient Global Impression of Severity (PGIS) of CSU at Week 12 and Week 24.
Time-to-event and proportion of patients receiving OCS for CSU during the planned treatment period.
Percentages of participants experiencing treatment-emergent adverse events (TEAEs) or serious adverse events (SAEs).
Incidence of treatment-emergent anti-drug antibodies (ADA) against dupilumab over time.

### Appropriateness of Measurements

The assessments used in this study are standard for the evaluation of therapy in participants with CSU. CSU is characterized by the recurrent formation of itchy hives, angioedema, or both for longer than 6 weeks (Zuberbier T, et al. Epidemiology of urticaria: a representative cross-sectional population survey. Clin Exp Dermatol. 2010; 35:869-73; Grob JJ, et al. Comparative study of the impact of chronic urticaria, psoriasis and atopic dermatitis on the quality of life. Br J Dermatol. 2005; 152:289-95.)

The primary endpoint is the change from baseline in weekly itch severity score (ISS7) at Week 24 (except EU and EU reference countries) and change from baseline in weekly urticaria activity score (UAS7, composite patient reported itch and hive score) at Week 24 for EU and EU reference countries. Itch is one of the most important patient relevant symptoms affecting quality of life in CSU and highly linked to how patients perceive their disease. ISS7 is one of the 2 components of urticaria activity score UAS7 (a composite score assessing both itch and hives) and an established and widely accepted patient-reported outcome tool to prospectively measure CSU activity (Mlynek A, et al. How to assess disease activity in patients with chronic urticaria? Allergy. 2008; 63:777-80.) that has been used in most clinical trials in CSU in the recent years as a main outcome parameter. (Maurer M, et al. Omalizumab for the treatment of chronic idiopathic or spontaneous urticaria. N Engl J Med. 2013;368(10):924-35; Casale TB, et al. Similar efficacy with omalizumab in chronic idiopathic/spontaneous urticaria despite different background therapy. J Allergy Clin Immunol Pract. 2015;3(5):743-50.)

Angioedema has been described as a highly prevalent clinical feature in CSU. Up to 40% of CSU patients can present with a mixed phenotype of urticarial and angioedema and 10% with angioedema alone. (Moolani Y, Lynde C, Sussman G. Advances in understanding and managing chronic urticaria [version 1; peer review: 2 approved]. F1000Res. 2016;5. pii: F1000 Faculty Rev-177. Available from: URL:https://doi.org/10.12688/f1000research.7246.1.) Angioedema Activity Score (AAS) a well-developed and adequately validated instrument to measure angioedema activity in CSU patients (Weller K, et al. Development, validation, and initial results of the Angioedema Activity Score. Allergy. 2013;68(9):1185-92.) was assessed to explore angioedema activity.

In addition to the UAS and angioedema, which provides a summary of signs and symptoms, it is important to get insights into patient self-assessment of disease control. To obtain a complete picture of the disease and assess its control over the course of treatment, a well-developed and validated instrument in CSU patients, the Urticaria Control Test, was used. (Weller K, et al. Development and validation of the urticaria control test: A patient-reported outcome instrument for assessing urticaria control. J Allergy Clin Immunol. 2014; 133:1365-72.)

Lastly, patients with CSU experience substantial HRQoL impairment. Therefore, the Dermatology Life quality Index (DLQI) or the children’s Dermatology Life Quality Index (CDLQI), 2 instruments developed to measure dermatology-specific quality of life in adult and in pediatric patients, respectively (Finlay AY, Khan GK. Dermatology life quality index (DLQI): a simple practical measure for routine clinical use. Clin Exp Dermatol. 1994; 19:210-6; Lewis-Jones MS, Finlay AY. The children’s dermatology life quality index(CDLQI): initial validation and practical use. Br J Dermatol. 1995; 132(6):942-9.), was assessed.

### Study Design

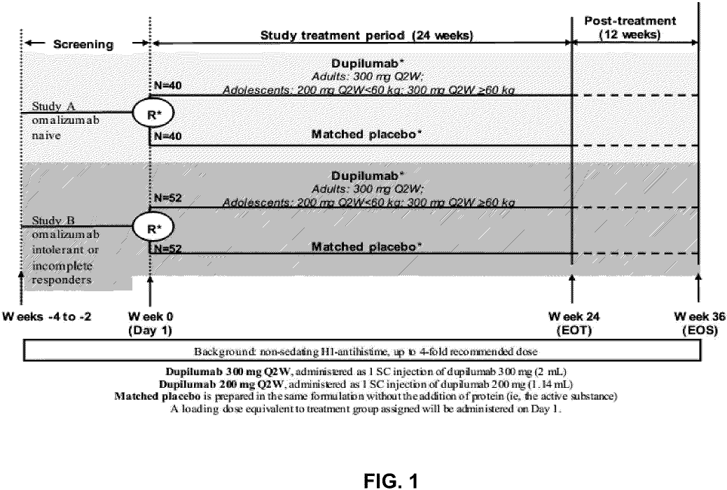

The overview of the study design is depicted in FIG. 1. The protocol comprises 2 studies of identical design, 1 in participants who are omalizumab naive (Study A) and 1 in participants who are intolerant or incomplete responders to omalizumab (Study B). Study A included adults, adolescents (≥12 to <18 years) and children (≥6 to <12 years in some selected countries). Study B included adults and adolescents. Both studies are 24-week, double-blind, randomized, placebo-controlled studies to evaluate the use of dupilumab in participants with CSU who remain symptomatic despite the use of H1-AH. The studies assessed the effect of dupilumab on the itch and hives scored separately once daily and averaged over 7 days and on the itch and hives frequency/severity through the urticaria activity score (composite) averaged over 7 days, on angioedema activity, urticaria control, and on patients' health-related quality-of-life (HRQoL), and health status.

Omalizumab incomplete responders are defined as patients treated with at least 300 mg omalizumab subcutaneous (SC) every 4 weeks (q4w) for at least 3 months (minimum of 3 injections) and who have had an inadequate response resulting in omalizumab discontinuation, as confirmed by Investigator assessment. Information about intolerance or incomplete response to omalizumab should be well documented in the patient’s medical records.

Both Studies A and B assessed the effect of dupilumab on the itch and hives frequency/severity scored individually and through the urticaria activity score (composite), on angioedema activity, urticaria control, and on participants' HRQoL and health status.

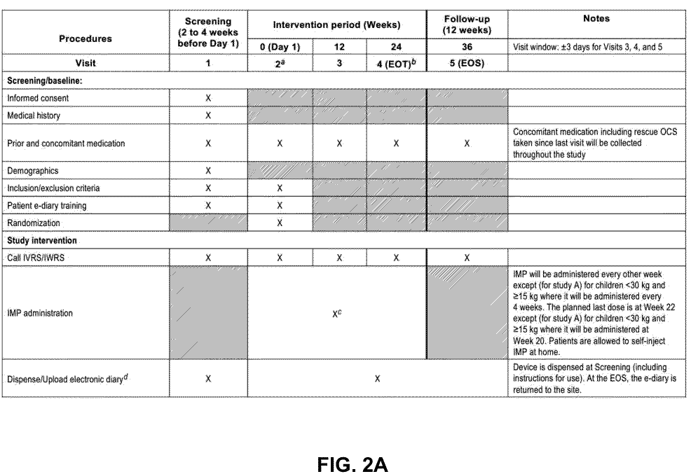

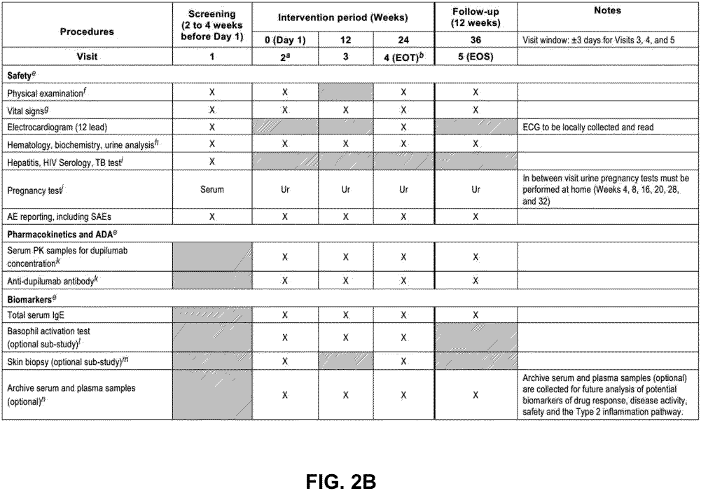

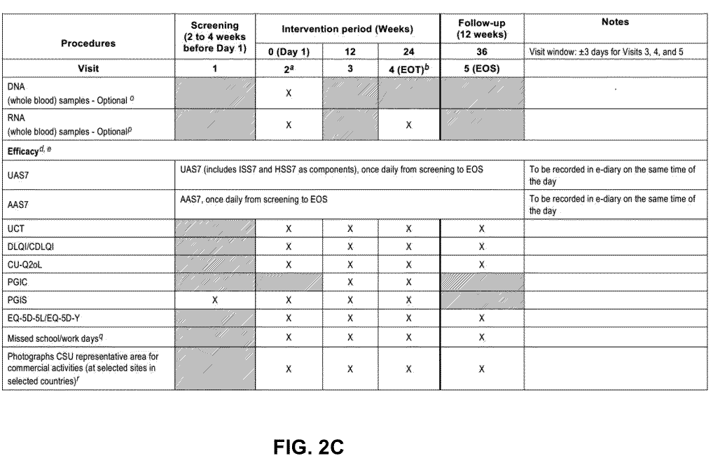

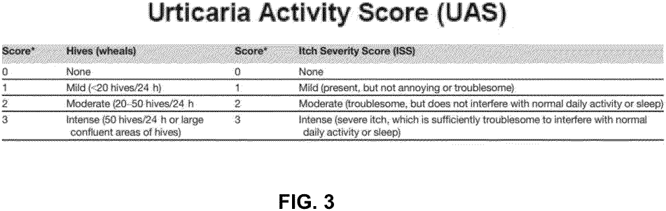

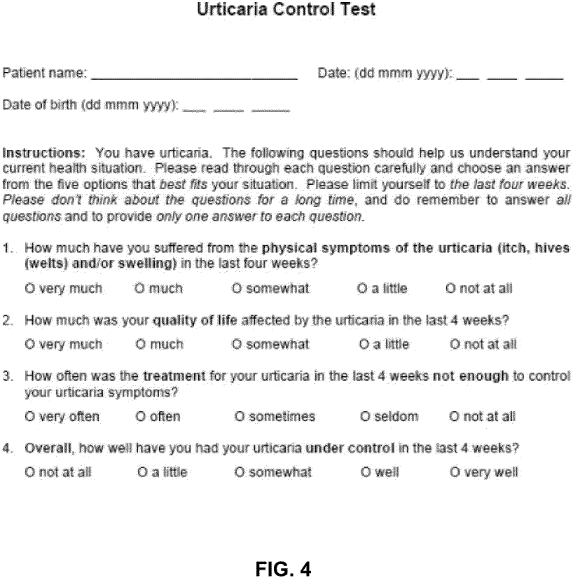

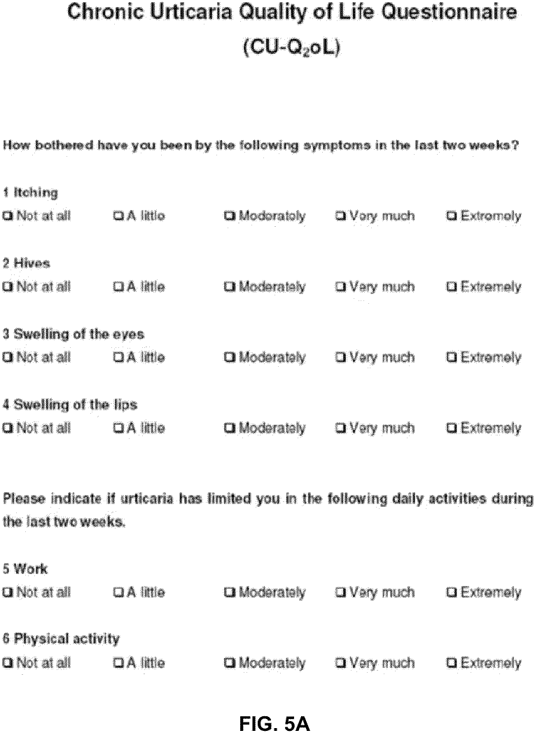

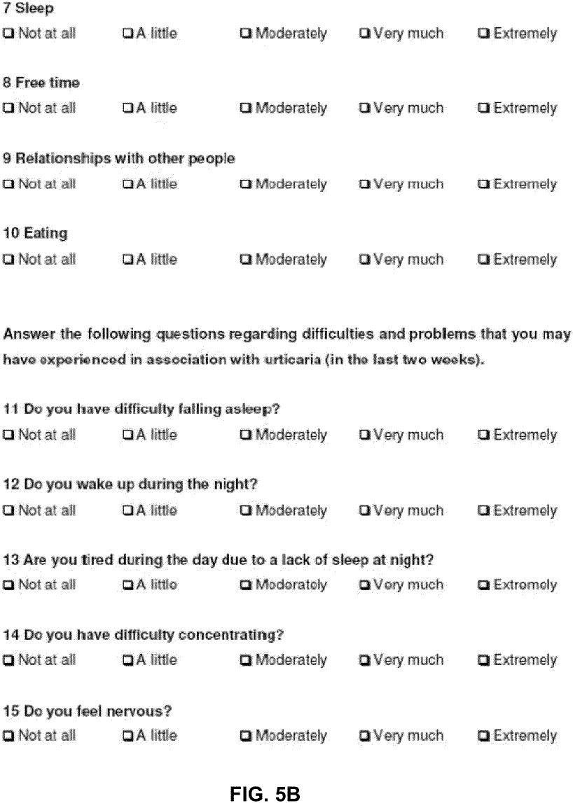

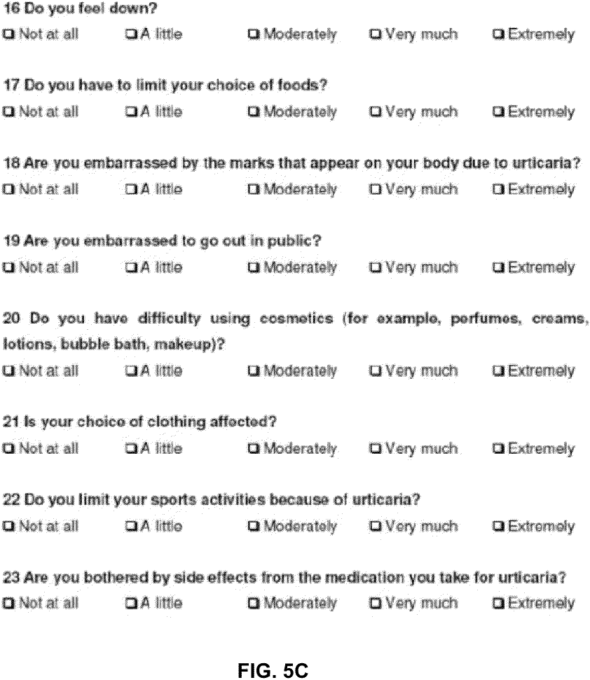

Each Study, A and B, is a parallel treatment study with 2 arms for each that is blinded/masked for participants and Investigators. While these are double blind trials with regard to the treatment with either dupilumab or placebo, they are not blinded to weight-based dose levels, due to the different volume size (2 mL versus 1.14 mL) of the dose level of dupilumab (300 mg/matching placebo or 200 mg/matching placebo) used for the different weight categories for adolescents and children ≥6 to <12 years of age. In addition, in children, the study is not blinded to dose regimen due to the different frequency of IMP administration (q4w versus q2w). The schedule of activities is depicted in FIGS. 2.

Each of the 2 (Studies A and B) consists of 3 periods:
Screening period (2 to 4 weeks).
IMP treatment period (24 weeks ±3 days): approximately 234 participants (130 participants in Study A and 104 participants in Study B) are randomized (1:1) to one of the following treatments:
   Dupilumab:
   Adults: 300 mg every 2 weeks (q2w);
   Adolescents: 200 mg q2w for adolescents <60 kg at screening or 300 mg q2w for ad adolescents ≥60 kg at screening;
   Study A: Children ≥6 to <12 years of age: 200 mg q2w for children ≥30 kg at screening and 300 mg q4w for children <30 kg and ≥15 kg at screening;
   Matched placebo.

A loading dose equivalent to treatment group assigned is administered on Day 1. Patients assigned to 300 mg q2w/q4w in dupilumab or matched placebo arm receive 2 injections of 2 mL on Day 1. Patients assigned to 200 mg q2w in dupilumab or matched placebo arm receive 2 injections of 1.14 mL on Day 1. Post-IMP treatment period (12 weeks ±3 days).

In each of the 2 studies, participants continued their established standard of care background therapy with a long-acting non-sedating H1-AH, at up to 4-fold the recommended dose. If patients were on a dose higher than 4-fold the recommended dose at screening visit (Visit 1), the Investigator could adjust the patient dose within the stipulated range at the screening visit (Visit 1). Patients continued to take the same daily dose throughout the study unless they experienced a flare for which rescue therapy could be initiated. All participants on 1- to 3-fold the approved H1-AH dose (maintenance dose used at screening) were allowed to take additional doses of their H1-AH medications as rescue therapy as long as they did not exceed 4-fold the recommended dose during the screening, treatment, and follow-up periods. If symptoms were still uncontrolled after increase of H1-AH to the maximum allowed dose, participants could take a short course of OCS as rescue therapy during the treatment and follow-up periods. The participants who already took 4-fold an approved H1-AH dose were allowed to take a short course of oral corticosteroids (OCS) as rescue therapy during the treatment and follow-up periods. However, for the purpose of the primary analysis, data collected after OCS use was set to missing and the worst postbaseline value before OCS was used.

### Scientific Rationale for Study Design

In each of the Studies A and B, a randomized, placebo-controlled study design where the effect of the IMP was assessed in CSU patients with moderate to severe symptoms on top of optimized background therapy was considered to be the most appropriate design to explore the efficacy and safety of dupilumab in participants with CSU who remain symptomatic despite the use of H1-AH and who are omalizumab naive or intolerant or incomplete responders.

Study A targeted omalizumab naive patients. More than 50% of CSU patients do not respond to H1 antihistamine treatment. (Zuberbier T, et al. The EAACI/GA2LEN/EDF/WAO guideline for the definition, classification, diagnosis and management of urticaria. Allergy. 2018;73(7): 1393-414; Zuberbier T, et al. The EAACI/GA(2) LEN/EDF/WAO guideline for the definition, classification, diagnosis, and management of urticaria: the 2013 revision and update. Allergy. 2014;69(7):868-87.)

Study B targeted omalizumab treated CSU patients. Approximately 20% to 40% patients do not respond to omalizumab and remain without an effective third-line treatment; these patients have the highest unmet medical need. (Zuberbier T, et al. The EAACI/GA2LEN/EDF/WAO guideline for the definition, classification, diagnosis and management of urticaria. Allergy. 2018;73(7): 1393-414.) The study also targeted omalizumab intolerant patients.

Including approximately 5% of adolescent patients in Study A and approximately 5% of adolescent patients in Study B is consistent with the omalizumab clinical development program and approximates the prevalence of adolescent patients with CSU. (Zuberbier T, Aberer W, Asero R, Bindslev-Jensen C, Brzoza Z, Canonica GW, et al. The EAACI/GA(2) LEN/EDF/WAO guideline for the definition, classification, diagnosis, and management of urticaria: the 2013 revision and update. Allergy. 2014;69(7):868-87.)

### End of Study Definition

A participant is considered to have completed the study if he/she has completed all phases of the study including the last end of study (EOS) Visit. If a participant discontinued a treatment period prematurely but completed follow-up to the planned EOS Visit, he/she was considered a completer. The overall EOS is defined as the date of the last visit of the last participant in the study.

### Study Population

### Inclusion Criteria

For each of the 2 Studies A and B, participants were eligible to be included in the study only if all of the following criteria apply:
Age
   I1. Study A and Study C: Participant must be ≥6 years to 80 years of age at the time of signing the informed consent. Study B: Participant must be ≥ 12 years (or the minimum legal age for adolescents in the country of the investigational site) to 80 years of age at the time of signing the informed consent
   Note: For those countries where local regulations do not permit enrollment of children aged ≥6 to <12 years, the recruitment is restricted to those who are ≥12 years of age (or the minimum legal age for adolescents in the country of the investigational site). For those countries where local regulations do not permit enrollment of children aged ≥6 to <12 years of age and adolescents, the recruitment is restricted to those who are ≥18 years of age.

### Type of Participant and Disease Characteristics

Participants who have a diagnosis of CSU refractory to H1-AH at the time of randomization, as defined by all of the following:
I 2. Diagnosis of CSU >6 months prior to screening visit (Visit 1).
I 3. The presence of itch and hives for >6 consecutive weeks at any time prior to screening visit (Visit 1) despite the use of H1-AH during this time period.
14. Participants using a study defined H1-AH for CSU treatment. Note: Participants should remain on their prescreening non-sedating H1-AH dose. Only up to 4-fold the recommended dose is allowed. If participants are on dose higher than 4-fold the recommended dose at screening, the Investigator can adjust the participant dose to the stipulated range at the screening visit (Visit 1). The H1-AH dose should be stable for at least 3 consecutive days prior to the screening visit (Visit 1).
I5. During the 7 days before randomization:
   UAS7 >16
   ISS7 >8
   Note: To be eligible for the study, participants must have no missing electronic diary (e-diary) (UAS7 and ISS7) in the 7 days before randomization.
I6. Study A (omalizumab naive): Participants who are omalizumab naive. Study B (omalizumab intolerant or incomplete responders): Omalizumab incomplete responders are defined as participants treated with at least 300 mg (q4w) omalizumab for at least 3 months (minimum of 3 injections) and who have had an inadequate response resulting in omalizumab discontinuation, as confirmed by Investigator assessment. Note: Information about intolerance or incomplete response to omalizumab should be well documented in the patient’s medical records.
I7. Participants must be willing and able to complete a daily symptom e-diary for the duration of the study.

### Study Overview

### Number of Participants

The total anticipated number of participants across the 2 studies was approximately 234 randomized participants.

For Study A, conducted in the omalizumab naïve population, approximately 130 participants were randomized. This corresponds to approximately 65 participants who were randomly assigned to each intervention arm. It was planned that approximately 5% of participants enrolled be adolescents and up to approximately 5% of participants enrolled be children ≥6 to <12 years of age (both children and adolescents recruited in some selected countries).

For Study B, conducted in the omalizumab intolerant or incomplete responder population, approximately 104 participants were randomized. This corresponds to approximately 52 participants who were randomly assigned to each intervention arm. It was planned that approximately 5% of participants enrolled be adolescents (recruited in some selected countries). An interim analysis was performed when the first 80 randomized patients completed their 24-Week treatment period, by the interim analysis cut-off date.

It was anticipated that approximately 30%-40% of enrolled participants have angioedema.

Omalizumab incomplete responders are defined as participants treated with at least 300 mg omalizumab every 4 weeks (q4w) for at least 3 months (minimum of 3 injections) and who have had an inadequate response resulting in omalizumab discontinuation, as confirmed by Investigator assessment.

### Intervention Groups and Duration

Patients who satisfied the inclusion and exclusion criteria were randomized (1:1) to 1 of the following investigational medicinal product (IMP) treatment groups:
Dupilumab:
   Adults: 300 mg every 2 weeks (q2w)
   Adolescents: 200 mg q2w for adolescents <60 kg at screening or 300 mg q2w for adolescents ≥60 kg at screening
   Study A (only): Children ≥6 to <12 years of age: 200 mg q2w for children ≥30 kg at screening and 300 mg q4 w for children <30 kg and ≥15 kg at screening
Matched placebo

Duration of study period (per participant)
Screening period (2 to 4 weeks)
Randomized IMP treatment period (24 weeks)
Post IMP treatment period (12 weeks)

Study Interventions Investigational medicinal product:
Dupilumab 300 mg and placebo matching dupilumab 300 mg supplied in prefilled syringes that are visually indistinguishable
Dupilumab 200 mg and placebo matching dupilumab 200 mg supplied in prefilled syringes that are visually indistinguishable

### Dupilumab

Formulation:
Dupilumab 300 mg: a 150 mg/mL dupilumab solution in a pre-filled syringe to deliver 300 mg in a 2 mL injection or
Dupilumab 200 mg: a 175 mg/mL dupilumab solution in a pre-filled syringe to deliver 200 mg in a 1.14 mL injection

### Route of Administration: Subcutaneous (SC) Injection

Dose regimen: 1 injection q2w/q4w after an initial loading dose (2 injections) on Day 1
Placebo:
   Formulation:

Placebo matching dupilumab 300 mg: identical formulation to the active 300 mg formulation without dupilumab, in a pre-filled syringe to deliver placebo in a 2 mL injection or
Placebo matching dupilumab 200 mg: identical formulation to the active 200 mg formulation without dupilumab, in a pre-filled syringe to deliver placebo in a 1.14 mL injection

### Route of Administration: SC Injection

Dose regimen:
1 injection q2w/q4w after an initial loading dose (2 injections) on Day 1

Participants continued their established standard of care background therapy with a long-acting non-sedating H1-AH, at up to 4-fold the recommended dose. If participants were on a dose higher than 4-fold the recommended dose at the screening visit (Visit 1), the Investigator could adjust the participant dose within the stipulated range at the screening visit (Visit 1). Participants continued to take the same daily dose throughout the study unless they experienced a flare for which rescue therapy could be initiated. The following list of H1-AH is allowed and noted with their recommended dose:
Cetirizine 10 mg once per day (qd).
Levocetirizine dihydrochloride 5 mg qd
Fexofenadine 60 mg twice per day or 180 mg qd
Loratadine 10 mg qd
Desloratadine 5 mg qd
Bilastine 20 mg qd
Rupatadine 10 mg qd
Other H1-AH after discussion with the Sponsor

### Study Intervention

Study intervention is defined as any investigational intervention(s), marketed product(s), placebo, or medical device(s) intended to be administered to a participant in Study A, Study B or Study C according to the study protocol. An overview of the study interventions administered is presented in Table 5 below.

TABLE 5

Overview of study interventions administered

| ARM name | Dupilumab | Placebo |
|---|---|---|
| Intervention name | For adults and adolescents ≥60 kg: Dupilumat 300 mg (q2w) | For adults and adolescents ≥60 kg: Placebo matching dupilumab 300 mg (q2w) |
| | For adolescents <60 kg and children ≥6 to <12 years of age ≥30 kg: Dupilumab 200 mg (q2w) | For adolescents <60 kg and children ≥6 to <12 years of age ≥30 kg: Placebo matching dupilumab 200 mg (q2w) |
| | For children ≥8 to < 12 years of age <30 kg and ≥15 kg: Dupilumab 300 mg (q4w)For adults and adolescents ≥60 kg: Dupilumab 300 mg (q2w) | For children ≥8 to <12 years of age <30 kg and ≥15 kg: Placebo matching dupilumab 300 mg (q4w)For adults and adolescents ≥60 kg: Placebo matching dupilumab 300 mg (q2w) |
| | For adolescents <60 kg and children ≥6 to <12 years of age ≥30 kg: Dupilumab 200 mg (q2w) | For adolescents <60 kg and children ≥6 to <12 years of age ≥30 kg: Placebo matching dupilumab 200 mg (q2w) |
| | For children ≥6 to <12 years of age <30 kg and ≥15 kg: Dupilumab 300 mg (q4w) | For children ≥6 to <12 years of age <30 kg and ≥15 kg: Placebo matching dupilumab 300 mg (q4w) |
| Type | Biological/Vaccine | Other |
| Dose formulation | Dupilumab 300 mg: a 150 mg/mL dupilumab solution in a pre-filled syringe to deliver 300 mg in 2 mL | Placebo matching dupilumab 300 mg will be supplied as an identical formulation to the active 300 mg formulation without dupilumab in a pre-filled syringe to deliver placebo in 2 mL |
| | or | or |
| Dupilumab 200 mg: a 175 mg/mL dupilumab solution in pre-filled syringe to deliver 200 mg in 1.14 mL | Placebo matching dupilumab 200 be supplied as an identical formul the active 200 mg formulation without dupilumab in a pre-filled syringe to deliver placebo in 114 mL mg will ation to | |
| Unit dose strength(s) | 300 mg or 200 mg | 0 mg |

| | | |
|---|---|---|
| ARM name | Dupilumab | Placebo |
| Dosage level(s) | 300 mg every 14 ±3 days after an initial loading dose of 600 mg | 0 mg every 14 ±3 days or 28 ±3 days after an initial loading dose of 0 mg |
| | or | |
| | 200 mg every 14 ±3 days after an initial loading dose of 400 mg | |
| | or | |
| | 300 mg every 28 ±3 days after an initial loading dose of 600 mg | |
| Route of administration | Subcutaneous | Subcutaneous |
| IMP and NIMP | IMP | IMP |
| Packaging and labeling | Each dose of dupilumab will be supplied as 1 glass pre-filled syringe packed in a patient kit box. Both glass pre-filled syringe and box will be labeled as required per country requirement | Each dose of placebo will be supplied as 1 glass pre-filled syringe packed in a patient kit box. Both glass pre-filled syringe and box will be labeled as required per country requirement |

During the 24-week treatment period, the investigational medicinal product (IMP) is administered every 14 ±3 days (q2w) or 28 ±3 days (q4W) for children <30 kg and ≥15 kg.

The Investigator or delegate trained the participant (or parent(s)/legally authorized representative/caregiver) how to prepare and inject IMP at Visit 2. The site staff injected the first dose of the 2 injections. The participant (or parent/legally authorized representative/caregiver) performed the second injection under the supervision of the Investigator or delegate.

When the participant has a study visit, the IMP is administered following clinical procedures and blood collection. Patients should be monitored for at least 30 minutes. The monitoring period may be extended as per country specific or local site-specific requirements.

If the participant (or parent/legally authorized representative/caregiver) is unable or unwilling to administer IMP, injections are performed at the site by way of unscheduled visits; or arrangements can be made for qualified site personnel and/or health care professionals (e.g., visiting nurse service) to administer IMP for the doses that are not scheduled to be given at the study site.

Subcutaneous injection sites should alternate between the upper thighs, 4 quadrants of the abdomen or the upper arms, so that the same site is not injected twice during consecutive administrations. Injection in the upper arms can only be done by a trained person (parent/legally authorized representative/caregiver trained by Investigator or Delegate) or health care professional but not the participants themselves. The IMP injection should be avoided in areas where patients have urticaria or angioedema.

Participant/parent/legally authorized representative/caregiver should be trained by the site staff to recognize potential signs and symptoms of hypersensitivity reaction in order to self-monitor/monitor at home for at least 30 minutes (or longer per country specific or local site-specific requirements) following injection. In case of hypersensitivity symptoms, the patient should contact healthcare provider/emergency.

For doses not given at the study site, paper diaries are provided to record information related to the injections. The paper diary is kept as source data in the patient’s study file.

### Non-Investigational Medicinal Product(s)

Participants should continue their established standard of care background therapy with a long-acting non-sedating H1-AH, at up to 4-fold the recommended dose. If participants are on a dose higher than 4-fold the recommended dose at the screening visit (Visit 1), the Investigator can adjust the participant dose within the stipulated range at the screening visit (Visit 1). Participants should continue to take the same daily dose throughout the study unless they experience a flare for which rescue therapy may be initiated. The following list of H1-AH is allowed and noted with their recommended dose:
Cetirizine 10 mg once per day (qd).
Levocetirizine dihydrochloride 5 mg qd
Fexofenadine 60 mg twice per day or 180 mg qd
Loratadine 10 mg qd
Desloratadine 5 mg qd
Bilastine 20 mg qd
Rupatadine 10 mg qd
Other H1-AH after discussion with the Sponsor

For other information related to H1-AH including safety precautions, please refer to the National Product labeling.

### Methods of Assigning Patients to Treatment Group

Patients were randomized in a 1:1 ratio treatment arm. The randomization was stratified first by age (adults versus adolescents versus children in Study A and adults versus adolescents in Study B; up to approximately 5% of total sample size for children in Study A and approximately 5% of total sample size for adolescents in Studies A and B, separately). In adults, randomization was stratified further by country. In adolescents/children ≥6 to <12 years of age, randomization was not stratified further.

It was anticipated that approximately 30%-40% of enrolled participants would have angioedema.

A randomized participant is defined as a participant who has been allocated to a randomized intervention regardless of whether the treatment was administered or not (i.e., participant registered by the IRT). A participant cannot be randomized more than once in the study.

### Methods of Blinding

Dupilumab 300 mg/200 mg and placebo matching dupilumab 300 mg/200 mg was provided in identically matched 2 mL/1.14 mL pre-filled syringes that are visually indistinguishable for each dose. Syringes and box are labeled with a treatment kit number. While these are double-blind trials with regard to the treatment with either dupilumab or placebo, they are not blinded to weight based dose levels, due to the different volume size (2 mL versus 1.14 mL) of the dose level of dupilumab (300 mg/matching placebo or 200 mg/matching placebo) that was used for the different weight categories for adolescents and children ≥6 to <12 years of age. In addition, in children, the study is not blinded to dose regimen due to the different frequency of IMP administration (q4w versus q2w).

### Concomitant Therapy

Any medication or vaccine (including over-the-counter or prescription medicines, vitamins, and/or herbal supplements) that the participant is receiving at the time of enrollment or receives during the study must be recorded along with:
Reason for use.
Dates of administration including start and end dates.
Dosage information including dose and frequency.

Long-acting non-sedating H1-AH, at up to 4-fold the recommended dose, are allowed as background medication and on demand as recue medication.

The concomitant use of the following therapies is prohibited during the entire study. Study treatment was to be discontinued in participants receiving these treatments:
Systemic immunosuppressants (immunosuppressive/immunomodulating drugs) e.g., systemic corticosteroids (oral or parenteral [intravenous, intramuscular, SC]), cyclosporine, mycophenolate-mofetil, interferon gamma, Janus kinase inhibitors, azathioprine, methotrexate, hydroxychloroquine, dapsone, sulfasalazine, colchicine, etc. Note: a short course of OCS is allowed as rescue therapy Antifibrinolytic tranexamic acid and epsilon-aminocaproic acid
Other monoclonal antibodies (which are biological response modifiers)
Phototherapy, including tanning beds
IVIG
Plasmapheresis
Other investigational drugs.

The concomitant use of following therapies is prohibited during the entire study, but study treatment did not need to be discontinued in participants receiving these treatments in violation of the protocol:
Topical corticosteroids
Topical calcineurin inhibitors
Topical and oral antihistamines (other than those allowed as background therapy)
Routine doses of doxepin (daily or every other day during 5 or more consecutive days)
LTRAs and H2 receptor antagonists, unless stable and taken for diseases other than CSU.

### Rescue Medicine

All participants on 1- to 3-fold the approved non-sedating H1-AH dose (maintenance dose used at screening) were allowed to take additional doses of their H1-AH medications as rescue therapy as long as they do not exceed 4-fold the recommended dose during the screening, treatment, and follow-up periods. If symptoms are still uncontrolled after increase of H1-AH to the maximum allowed dose, participants could take a short course of OCS as rescue therapy during the treatment and follow-up periods. The participants on stable dose of 4-fold the approved H1-AH dose were allowed to take a short course of OCS as rescue therapy during the treatment and follow-up periods. In order to ensure consistency, when possible, it is recommended to use OCS for 5 to 7 days with a starting dose of oral prednisone 40 mg (or clinically comparable OCS) followed by taper per the Investigator’s judgment.

The initial maintenance antihistamine dose should remain stable throughout the study, and participants should continue their maintenance dose once rescue treatment is no longer required.

The use of permitted rescue medications should be delayed, if possible, for at least 8 weeks following the initiation of the investigational treatment. The date and time of rescue medication administration as well as the name and dosage regimen of the rescue medication must be recorded.

For other information related to H1-AH and OCS including safety precautions please refer to the National Product labeling.

### Discontinuation of Study Intervention

In rare instances, it may be necessary for a participant to permanently discontinue study intervention. If study intervention is permanently discontinued, the participant should complete early treatment discontinuation visit with all assessments planned for the end of treatment (EOT) Visit.

The participants may withdraw from treatment with the IMP if he or she decides to do so, at any time and irrespective of the reason, or this may be the Investigator’s decision. All efforts should be made to document the reason(s) for treatment discontinuation and this should be documented in the eCRF.

Patients must be permanently withdrawn from the study treatment for the following reasons:
At their own request or at the request of their legally authorized representative (legally authorized representative means an individual or judicial or other body authorized under applicable law to consent on behalf of a prospective participant to the patient’s participation in the procedure(s) involved in the research).
If, in the Investigator’s opinion, continuation in the study would be detrimental to the participant’s well-being.
At the specific request of the Sponsor.
In the event of a protocol deviation, at the discretion of the Investigator or the Sponsor.
Any code broken requested by the Investigator will lead to permanent discontinuation of study intervention.
Pregnancy.
Anaphylactic reactions or systemic allergic reactions that are related to IMP and require treatment.
Diagnosis of a malignancy during study, excluding carcinoma in situ of the cervix, or squamous or basal cell carcinoma of the skin.
Any opportunistic infection or other infections whose nature or course may suggest an immunocompromised status.
Serum alanine aminotransferase (ALT) >3 × Upper Limit of Normal (ULN) and total bilirubin >2 × ULN.
Serum ALT >5 × ULN if baseline ALT ≤2 × ULN or ALT >8 × ULN if baseline ALT >2 × ULN.
If the participant develops a medical condition that requires use of prohibited medication.

### Efficacy Assessments

Efficacy data was collected via electronic devices. The e-diary is used for daily recording of PRO such as the UAS7 and AAS7 questionnaires, and use of H1-AH medication. This device was dispensed at screening visit (Visit 1), including instructions for use and participant(s)/parent(s)/caregiver(s)/legally authorized representative(s) were instructed on the use of the device. Recorded information was downloaded from this device daily. At the EOS visit, the e-diary was downloaded and returned to the site. On regular basis, the site staff should review on vendor’s website the information downloaded from participants’ e-diary. They should particularly check status of the disease reviewing UAS7 and AAS7, as well as compliance to background therapy and overall e-diary compliance. The site should follow-up with the subject as appropriate. The same questionnaires as for adolescents aged <16 years were used for children aged 6 to <12 years. For UCT, DLQI (≥16 yearsold)/CDLQI (≥6 to <16 years old), CU-Q2oL, 5-level EuroQol 5-dimensional questionnaire (EQ-5D-5L) (16 years old)/EuroQol 5-dimensional questionnaire youth (EQ-5D-Y) (6 to <16 years old), PGIC, PGIS, and missed school/work days questionnaires, participant(s) filled in the questionnaires during their site visit on a tablet that was provided to the site. This device is kept at the site during the study.

### Urticaria Activity Score

The Urticaria Activity Score (UAS) is a validated patient-recorded outcome (PRO) measure. The daily UAS is the sum of the daily Hive Severity Score (HSS, ranging from 0 = None to 3 = more than 50 hives) and the daily Itch Severity Score (ISS, ranging from 0 = None to 3 = intense), the 2 key urticaria signs and symptoms which are wheals and itch. The daily UAS scores range from 0 to 6 point/day. Daily UAS scores are summed over 7-day period to create the UAS7, ranging from 0 to 42, and is composed of the HSS7 and ISS7 components. The UAS7 is an established and widely accepted PRO tool to prospectively measure CSU activity. (Mlynek A, et al. How to assess disease activity in patients with chronic urticaria? Allergy. 2008;63(6):777-80.) It has been used in most clinical trials in CSU in the recent years as a main outcome parameter and medical practice (Maurer M, et al. Omalizumab for the treatment of chronic idiopathic or spontaneous urticaria. N Engl J Med. 2013;368(10):924-35; Casale TB, et al. Similar efficacy with omalizumab in chronic idiopathic/spontaneous urticaria despite different background therapy. J Allergy Clin Immunol Pract. 2015;3(5):743-50). A minimal important difference (MID) value ranging from 9.5 to 10.5 has been defined to help interpretation of the change in score in CSU participants. (Hollis K, et al. Comparison of urticaria activity score over 7 days (UAS7) values obtained from once-daily and twice-daily versions: Results from the ASSURE-CSU study. Am J Clin Dermatol. 2018;19(2):267-74; Hawro T, et al. The urticaria activity score-validity, reliability, and responsiveness. J Allergy Clin Immunol Pract. 2018;6(4):1185-90; Mathias SD, et al. Evaluating the minimally important difference of the urticaria activity score and other measures of disease activity in patients with chronic idiopathic urticaria. Ann Allergy Asthma Immunol. 2012;108(1):20-4.)

### Angioedema Activity Score

The Angioedema Activity Score (AAS) is a validated PRO measure that assesses angioedema activity. (Weller K, et al. Development, validation, and initial results of the Angioedema Activity Score. Allergy. 2013;68(9):1185-92.) The AAS is a diary in which participants document on a daily basis the presence or absence of angioedema during the past 24 hours. If angioedema is present, participants answer 5 additional questions about the time of the day the swelling episode occurred, and the severity and impact on daily functioning and appearance this swelling episode has had. Each AAS item is scored between 0 and 3 points, that is, the minimum and maximum daily AASs are 0 and 15 points. The daily AASs are summed up to 7-day scores (AAS7), with 7-day scores ranging from 0 to 105 (Id). A MID of the AAS7 of around 8 points has been established (Id).

### Urticaria Control Test

The Urticaria Control Test (UCT) is a validated PRO measure for assessing urticaria control (Weller K, et al. Development, validation, and initial results of the Angioedema Activity Score. Allergy. 2013;68(9):1185-92.) based on 4 items: severity of pruritus and wheals urticaria symptoms; frequency of treatment being not sufficient; QoL impairment; overall urticarial control). Each item is rated on a 5-point Likert-type scale (scored with 0 to 4 points). Low scores indicate high disease activity and low disease control. The UCT total score is calculated by adding all 4 individual item scores. Accordingly, the minimum and maximum UCT scores are 0 and 16, with a score of 16 points indicating complete disease control. (Weller K, et al. Development, validation, and initial results of the Angioedema Activity Score. Allergy. 2013;68(9):1185-92.)

### Dermatology Life Quality Index and Children’s Dermatology Quality Life Quality Index

The Dermatology Life Quality Index (DLQI) is a PRO developed to measure dermatology-specific HRQoL in adult participants. (Finlay AY, Khan GK. Dermatology life quality index (DLQI): a simple practical measure for routine clinical use. Clin Exp Dermatol.1994;19:210-6.) The instrument comprises 10 items assessing the impact of skin disease on participants' HRQoL over the previous week. The items cover symptoms, leisure activities, work/school or holiday time, personal relationships including intimate, the side effects of treatment, and emotional reactions to having a skin disease. It is a validated questionnaire used in clinical practice and clinical trials> (Chernyshov PV. The evolution of quality of life assessment and use in dermatology. Dermatology. 2019;235(3):167-74.) Response scale is a 4-point Likert scale (0 = “not at all” and 3 = “very much”) for 9 items. The remaining 1 item about work/studying asks whether work/study has been prevented and then (if “No”) to what degree the skin condition has been a problem at work/study; the item is rated on a 3-point Likert scale (‘Not at all’ to ‘A lot’). Overall scoring ranges from 0 to 30, with a high score indicative of a poor HRQoL. Using an integrated analysis of distribution and anchor-based approaches using the change in DLQI total score and participant-assessed itch severity scores, the MID for the DLQI in participants with chronic idiopathic urticaria was reported to be in the range of 2.24 to 3.10 points. (Shikiar R, et al. Minimal important difference (MID) of the dermatology life quality index (DLQI): results from patients with chronic idiopathic urticaria. Health Qual. Life Outcomes. 2005; 3:36.)

The Children’s Dermatology Quality Life Quality Index (CDLQI) is a validated questionnaire designed to measure the impact of skin disease on children’s HRQoL. (Lewis-Jones MS, Finlay AY. The children’s dermatology life quality index(CDLQI): initial validation and practical use. Br J Dermatol.1995;132(6):942-9.) Patients provide responses to 10 questions (symptoms feelings associated with disease, the impact of the disease on leisure, school or holidays, personal relationships, sleep, and side effects of treatment for the skin disease). The instrument has a recall period of 7 days. Nine of the 10 questions are scored on a 4-point Likert scale ranging from 0 = not at all/question unanswered to 3 = very much. Question 7 has an additional possible response (prevented school), which is assigned a score of 3. The CDLQI total score is the sum of the score of each question with a maximum of 30 and a minimum of 0. The higher the score, the greater the impact is on the child’s HRQoL. Patients complete the DLQI (≥16 years old) or CDLQI (≥12 - <16).

### Chronic Urticaria Quality of Life Questionnaire

The CU-Q2oL is a disease-specific instrument used to assess the QoL in adult participants with CSU. (Baiardini I, et al. A new tool to evaluate the impact of chronic urticaria on quality of life: chronic urticaria quality of life questionnaire (CU-QoL). Allergy. 2005;60(8): 1073-8.) The CU-Q2oL is a 23-item, self-administered questionnaire that includes 6 QoL dimensions: pruritus, swelling, impact on life activities, sleep problems, limits, and looks. Each item is scored on a 5-point Likert scale (1 = not at all, 5 = extremely) where participants indicate how troubled they are within each dimension. The individual items are summed to generate the overall CU-Q2oL score, which is then converted to a 0 to 100 scale; higher scores indicate greater QoL impairment.

### Patient Global Impression of Change of CSU Disease and Patient Global Impression of Severity of CSU Disease

The Patient Global Impression of Change (PGIC) is a 1-item questionnaire that asks the participant to provide the overall self-assessment of change in their CSU on a 7-point scale, compared to just before participant started taking the study treatment. Response choices are: 0 = “very much better,” 1 = “moderately better,” 2 = “a little better,” 3 = “no change,” 4 = “a little worse,” 5 = “moderately worse,” 6 = “very much worse.”

The Patient Global Impression of Severity (PGIS) is a 1-item questionnaire that asks participants to provide the overall self-assessment of their participant’s disease severity on a 4-point scale for the past week. Response choices are: 1 = “none,” 2 = “mild,” 3 = “moderate,” 4 = “severe.” Patients complete the PGIC and PGIS.

### EuroQol 5 Dimensions Questionnaire

The Euroqol-5 dimensions (EQ-5D) is a standardized PRO measure of health status developed by the EuroQol Group in order to provide a simple, generic measure of health for clinical and economic appraisal. The adult version of the questionnaire is adapted to patients aged 16 and older. The EQ-5D consists of 2 parts: the descriptive system and the EQ visual analogue scale (EQ VAS). The EQ-5D 5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “inability to do the activity.” (Herdman M, et al. Development and preliminary testing of the new five-level version of EQ-5D (EQ-5D-5L). Qual. Life Res. 2011;20(10):1727-36.) The respondent is asked to indicate his/her health state by ticking (or placing a cross) in the box against the most appropriate statement in each of the 5 dimensions. This results in a 1-digit number expressing the level for that dimension. The digits for 5 dimensions can be combined in a 5-digit number describing the respondent’s health state. The EQ VAS records the respondent’s self-rated health on a vertical, VAS where the endpoints are labeled “best imaginable health state (100)” and “worst imaginable health state (0).” This information can be used as a quantitative measure of health outcome as judged by the individual respondents.

The EQ-5D Youth version (EQ-5D Y) is administered to children ≥6 to <12 years old and adolescents 12 to 15 years old. (Wille N, et al. Qual. Life Res. 2010;19(6):875-86.) The EQ-5D-Y is based on the EQ-5D-3L and essentially consists of 2 pages: the EQ-5D descriptive system and the EQ VAS. The EQ-5D-Y descriptive system comprises the following 5 dimensions: mobility, looking after myself, doing usual activities, having pain or discomfort and feeling worried, sad or unhappy. Each dimension has 3 levels: no problems, some problems and a lot of problems. The EQ VAS records the younger patient’s self-rated health on a vertical VAS where the endpoints are labelled “the best health you can imagine” and “the worst health you can imagine.” Patients complete the EQ-5D Y or EQ-5D questionnaire.

### Missed School/Work Days

Patients who are employed or enrolled in school were asked to report the number of sick leave/missed school days since the last study assessment.

### Safety Assessments

Physical examinations
A complete physical examination includes skin, nasal cavities, eyes, ears, respiratory, cardiovascular, gastrointestinal, neurological, lymphatic, and musculoskeletal systems.
Investigators should pay special attention to clinical signs related to previous serious illnesses.
Any new finding or worsening of previous finding should be reported as a new adverse event.

Vital signs
Vital signs are measured in a semi-supine or sitting position after 5 minutes rest and include axillary or oral temperature (same method of temperature measurement should be used during the course of the study), systolic and diastolic blood pressure, and pulse and respiratory rate. Blood pressure and pulse measurements should be assessed using the same arm with a completely automated device. Manual techniques are used only if an automated device is not available.
Body weight (kg) is measured at screening (Visit 1) and at EOT/EOS visits. Height is measured at screening visit (Visit 1). Height and weight are measured with indoor clothing but without shoes.

Electrocardiograms
Single standard 12-lead ECG are obtained using an ECG machine that automatically calculates the heart rate and measures PR, QRS, QT, and QTc intervals. The ECG should be recorded after 10 minutes of rest in the supine position.

Clinical safety laboratory assessments
The Investigator must review the laboratory report, document this review, and record any clinically relevant changes occurring during the study in the AE section of the CRF. The laboratory reports must be filed with the source documents. Clinically significant abnormal laboratory findings are those which are not associated with the underlying disease, unless judged by the Investigator to be more severe than expected for the participant’s condition.
All laboratory tests with values considered clinically significantly abnormal during participation in the study should be repeated until the values return to normal or baseline or are no longer considered clinically significant by the Investigator or Medical Monitor.
If such values do not return to normal/baseline within a period of time judged reasonable by the Investigator, the etiology should be identified, and the Sponsor notified.
If laboratory values from non-protocol-specified laboratory assessments performed at the institution’s local laboratory require a change in participant management or are considered clinically significant by the Investigator (e.g., SAE or AE or dose modification), then the results must be recorded in the CRF.

### ADVERSE EVENTS AND SERIOUS ADVERSE EVENTS

### Adverse Event of Special Interest

An adverse event of special interest (AESI) is an AE (serious or non-serious) of scientific and medical concern specific to the Sponsor’s product or program, for which ongoing monitoring and immediate notification by the Investigator to the Sponsor is required. Such events may require further investigation in order to characterize and understand them. Adverse events of special interest may be added, modified or removed during a study by protocol amendment.

For these AESIs, the Sponsor is informed immediately (i.e., within 24 hours), per SAE notification, even if not fulfilling a seriousness criterion, using the corresponding pages in the CRF (to be sent) or screens in the e-CRF.
Anaphylactic reactions
Systemic hypersensitivity reactions
Helminthic infections
Any severe type of conjunctivitis or blepharitis
Keratitis
Clinically symptomatic eosinophilia (or eosinophilia associated with clinical symptoms)
Significant ALT elevation
   ALT >5 × the ULN in participants with baseline ALT ≤2 × ULN; or
   ALT >8 × ULN if baseline ALT >2 × ULN.
Pregnancy of a female subject entered in a study as well as pregnancy occurring in a female partner of a male subject entered in a study with IMP/non-investigational medicinal product (NIMP)
   Pregnancy occurring in a female participant entered in the clinical trial or in a female partner of a male participant entered in the clinical trial. It is qualified as an SAE only if it fulfills 1 of the seriousness criteria.
   In the event of pregnancy in a female participant, IMP should be discontinued.
   Follow-up of the pregnancy in a female participant or in a female partner of a male participant is mandatory until the outcome has been determined.
   Abnormal pregnancy outcomes (e.g., spontaneous abortion, fetal death, stillbirth, congenital anomalies, ectopic pregnancy) are considered SAEs.
Symptomatic overdose (serious or non-serious) with IMP/NIMP
   An overdose (accidental or intentional) with the IMP is an event suspected by the Investigator or spontaneously notified by the participant and defined as at least twice the intended dose during an interval of less than 11 days. The circumstances (i.e., accidental or intentional) should be clearly specified in in the overdose form.
   An overdose (accidental or intentional) with any NIMP is an event suspected by the Investigator or spontaneously notified by the participant and defined as at least twice the maximum prescribed daily dose, within the intended therapeutic interval. “The circumstances (i.e., accidental or intentional) should be clearly specified in the overdose form.”

Adverse events are by the participant (or, when appropriate, by a caregiver, parent, surrogate, or the participant’s legally authorized representative). The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

### Pharmacodynamics

IgE is the only pharmacodynamic parameter that was evaluated in this study.

### Sample Size Determination

The total anticipated number of participants across the 3 studies is approximately 384 randomized participants.

### Populations for Analyses

For purposes of analysis which are conducted separately for each study (A, B and C), the following populations were defined separately for each study (A, B and C) (Table 6):

TABLE 6

Populations for Analyses

| Population | Description |
|---|---|
| Screened | All participants who sign the ICF. |
| Randomized | The randomized population includes all patients with a treatment kit number allocated and recorded in the IRT datebase, and regardless of whether the treatment kit was used or not. Patients treated without being randomized will not be considered randomized and will not be included in any efficacy population. |
| Intent-to-treat (ITT) Intent-to-treat24 (ITT24) (For Study B interim analysis) | All randomized patients analyzed according to the treatment group allocated by randomization. All participants who were randomized at least 24 weeks before the interim analysis cut-off date and would have completed the 24 visit by the cut-off interim analysis. Week date of The exact interim analysis cut-off date will be specified in the SAP , which will be finalized before the interim analysis. |
| Efficacy Safety | The ITT population (ITT24 for Study 8 interim analysis). All participants randomly assigned to study intervention and who take at least 1 dose of study intervention. Participants will be analyzed according to the intervention they actually received. Randomized participants for whom it is unclear whether they took the study medication will be included in the safety population as randomized. For participants who accidentally receive different treatment from the planned, the actual intervention allocation for as-treated analysis will be the dupilumab group. The pharmacodynamic (PD) analyses will be performed on the safety population. |
| Population | Description |
| Pharmacokinetic (PK) | The PK population includes all participants in the safety population with at least one non-missing result for functional dupilumab concentration in serum after first dose of the study treatment. Patients will be analyzed according to the intervention actually received. |
| Antidrug antibody (ADA) | ADA population includes all participants in the safety population who have at least one non-missing ADA result after first dose of the study treatment. Patients will be analyzed according to the intervention actually received. |

Abbreviations: ADA = antidrug anti ibody; ICF = Informed consent form, IRT = interactive response technology; PD = Pharmacodynamic

### Statistical Analyses

This section is a summary of the statistical analyses of the primary and secondary endpoints. Table 7 below depicts efficacy analyses.

TABLE 7

Efficacy Analyses

| Endpoint | Statistical analysis methods |
|---|---|
| Primary Primary endpoint Change from baseline in IS87 at Week 24 (except EU and EU reference countries) Primary endpoint for EU and EU reference countries: Change from baseline in UA87 at Week 24 | The primary efficacy endpoint will be analyzed using a hybrid method of the worst-observation carried forward (WOCF) and multiple imputation. For patients taking selected prohibited medications and/or rescue medications (details of selection will be specified in the statistical analysis plan), their data after the medication usage will be set to missing, and the worst postbaseline value on or before the time of the medication usage will be used to impute missing Week 24 value (for patients whose postbaseline values are all missing, the baseline will be used to impute). Patients who discontinue the treatment prematurely are encouraged to follow the planned clinical visits and in these patients who did not take the selected prohibited medications and/or rescue medications, all data collected after treatment discontinuation will be used in the analysis. For these participants, been tried to missing data may still happen despite all efforts have collect the data after treatment discontinuation. For participants who discontinue due to lack of efficacy, all data collected after discontinuation will be used in this analysis, and a WOCF approach will be used to impute missing Week 24 value if needed. For participants who discontinue not due to lack of efficacy, a multiple imputation approach will be used to impute missing Week 24 value, and this multiple imputation will use all participants excluding participants who have taken the selected prohibited medications and/or rescue medications on or before Week 24 and excluding patients who discontinue due to lack of efficacy on or before Week 24. Each of the imputed complete data will be analyzed by fitting an analysis of covariance model with the baseline value of the primary endpoint, treatment group, presence of angioederna at baseline, and region (combined countries) as |
| Endpoint | Statistical analysis methods |
| | covariates. Statistical inference obtained from all imputed data will be combined using Rubin’s rule. Descriptive statistics including number of subjects, mean, standard error, and least squares (LS) mean changes (and standard error) score will be provided. In addition, difference of the dupilumab group against placebo in LS means and the corresponding 95% confidence intervals (CI) will be provided along with the p-values. Sensitivity analysis Tipping point analyses and other sensitivity analyses will be performed to confirm robustness of the results with respect to the missing data handling. Details of the sensitivity analyses will be provided in the SAP. Subgroup analysis To assess the consistency in treatment effects across different subgroup levels, subgroup analyses will be performed for the primary efficacy endpoint with respect to age group, gender, region, and other factors that will be specified in the SAP. |
| Secondary Change from baseline in UAS7 at Week 12 and Week 24 (except EU and EU reference countries) Change from baseline in HSS7 at Week 12 and Week 24 Change from baseline in ISS7 at Week 12 and Week 24 (in EU and EU reference countries) Change from baseline in AAS7 at Week 12 and Week 24 Change from baseline in UCT at Week 12 and Week 24 Change from baseline in DLQI/CDLQI at Week 12 and Week 24 PGIC at Week 12 and Week 24 Change from baseline in PGIS, at Week 12 and Week 24 Change from baseline in ISS at all time points | These endpoints will be analyzed using the same approach as for the primary endpoint of change from baseline in ISS7 at Week 24. Detailed analyses will be described in the SAP finalized before database lock. |
| Secondary Proportion of ISS7 MID responders at Week 12 and Week 24 Proportion of patients with UAS7 ≤6 at Week 12 and Week 24 Proportion of patients with UAS7 = 0 at Week 12 and Week 24 Proportion of well-controlled patients (UCT ≥12 at Week 12 and Week 24 | These responder endpoints will be analyzed using the Cochran-Mantel-Haenszel test adjusted by baseline disease severity, presence of angioedema at baseline, and region. The baseline disease severity will be defined according to UAS7 <28 or ≥28. Comparisons of the response rates between dupilumab dose and placebo will be derived. Patients who receive selected prohibited medications and/or rescue medications will be considered as non-responders for time points after the medication usage. For other participants, all available data including those collected during the off-treatment period will be used to determine the responder/non-responder status Missing data will be considered as non-responders. |
| Secondary Time to ISS7 MID response Proportion and time to event of patients receiving OCS for CSU during the planned treatment period | These time-to-event endpoints will be analyzed using the Cox proportional hazards model, including treatment, the corresponding baseline value, presence of angioedema at baseline, and region as covariates. The estimates of the hazard ratio between dupilumab dose and placebo will be derived |
| Exploratory | Will be described in the SAP finalized before database lock. |

### Safety Analyses

All safety analyses were performed on the safety population. The summary of safety results are presented by treatment group. The baseline value is defined generally as the last available value before randomization. The safety analyses are summarized in Table 8 below.

TABLE 8

Safety Analyses

| | |
|---|---|
| Endpoint | Statistical Analysis Methods |
| AE SAE, AE leading to death, AE leading to permanent treatment discontinuation | Adverse event incident tables will present by system organ class (SOC) (sorted by internationally agreed order), high-level group term (HLGT), high level term (HLT) and preferred term (PT) sorted in alphabetical order for each treatment group, the number (n) and percentage (%) of participants experiencing an AE. Multiple occurrences of the same event in the same participant will be counted only once in the tables within a treatment phase. The denominator for computation of percentages is the safety population within each treatment group. Proportion of participants with at least 1 treatment-emergent adverse event (TEAE), treatment-emergent SAE. TEAE leading to death, and TEAE leading to permanent treatment discontinuation tabulated by treatment group. In addition, TEAEs will be descibed according to maximum intensity and relation to the study intervention. Serious AEs and AEs leading to study discontinuation that occur outside the treatment-emergent period will be summarized separately. |
| AESI and other AE groupings | Incidence of each type of AESI and other AE groupings will be tabulated by treatment group. For each type of AESI, the following analysis will be generated. • A summary of the number (%) of participants with - Any TEAE - Any SAE (regardless of treatment-emergent status) - Any treatment-emergent SAE - Any AE leading to death - Any TEAE leading permanent treatment discontinuation - Any TEAE related to study inervention reported by the investigator - Any TEAE by maximum intensity, corrective treatment, and final outcome • Kaplan-Meier (K-M) estimates of probability of having at least one TEAE at specific time points, and K-M curve to depict the course of event onset ever time. The method to identity AESIs and other AE groupings will be specified in the SAP. |
| Endpoint | Statistical Analysis Methods |
| Death | The following deaths, summaries will be generated: • Number (%) of participants who died by study period (TEAE on-study) summarized on the safety population by treatment received. • Death in nonrandomized participants or randomized and not treated participants. • TEAE leading to death (death as an outcome on the AE eCRF page as reported by the Investigator) by primary SOC, HLGT, HLT, and PT showing number (%) of participants sorted by internationally agreed order of SOC and alphabetic order of HLGT, HLT, and PT. |
| Laboratory parameters | Results and change from baseline for the parameters will be summarized by treatment group for baseline and each post baseline time point, endpoint, minimum and maximum value. Summary statistics will include, number of participants, mean, standard deviation, median, Q1, Q3, minimum, and maximum. The following definitions will be applied to laboratory parameters • The potentially clinically significant abnormality (PCSA) values are defined as abnormal values considered medically important by the Sponsor according to predefined criteria/thresholds based on literature review and defined by the Sponsor far clinical taboratory tests. • PCSA criteria will determine which participants had at least 1 PCSA during the on-treatment period, taking into account all evaluations performed during the on-treatment period, including unscheduled or repeated evaluations. The number of all such participants will be the numerator for the on-treatment PCSA percentage. The proportion of participants who had at least one incidence of PCSA at any time during the treatment emergent period will be summarized by treatment group. Shift tables showing changes with respect to the baseline status will be provided. |

### Summary of Study A Methods, Results, and Conclusion

Methods: LIBERTY-CSU CUPID Study A (NCT04180488), a randomized, placebo -controlled, 24-week, phase 3 trial evaluated dupilumab efficacy and safety in patients aged ≥6 years with CSU who remained symptomatic despite treatment with antihistamines. Patients on a standard or ≤4-fold dose of antihistamines were randomized to receive add-on dupilumab (n=70) 300 mg (adults/adolescents ≥60 kg) or 200 mg (adolescents <60 kg/children ≥30 kg), or matching placebo (n=68) subcutaneously every 2 weeks. Primary and key secondary endpoints included change from baseline at Week 24 in Itch Severity Score over 7 days (ISS7) and Urticaria Activity Score over 7 days (UAS7). Other secondary endpoints included change from baseline at Week 24 in Hive Severity Score over 7 days (HSS7).

### Results Study A

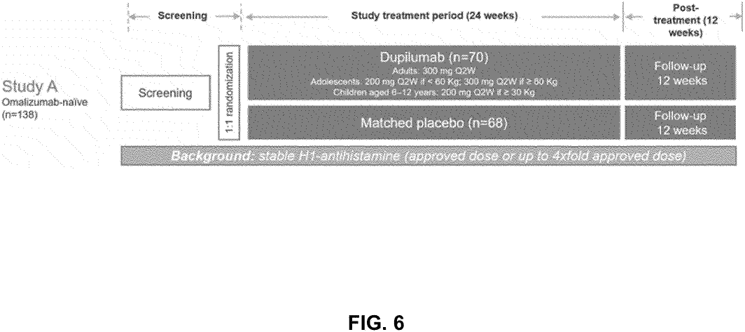

As depicted in FIG. 6, Study A included participants who were omalizumab naive who were treated with dupilumab for 24 weeks.

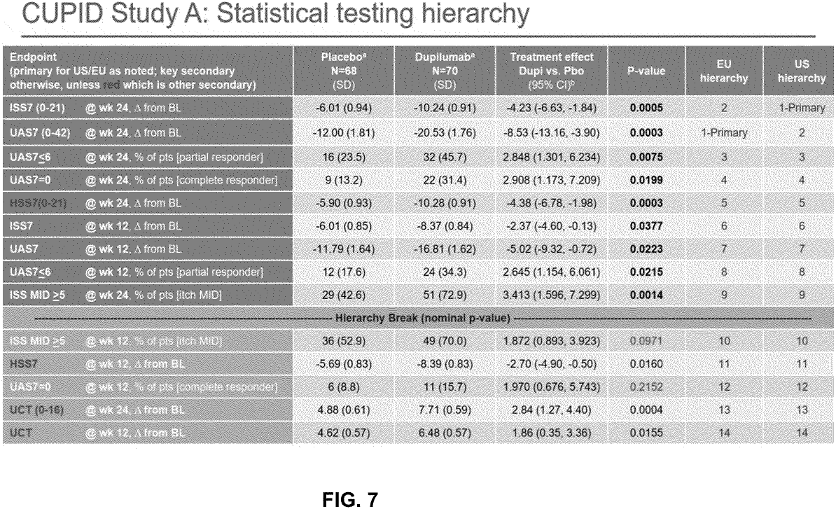

The statistical testing hierarchy for Study A is summarized in FIG. 7. The p values for the primary endpoints at 12 and 24 weeks are presented in this figure.

Baseline characteristics were generally balanced across treatment groups. Mean ISS7, UAS7, and HSS7 (dupilumab/placebo) at baseline were: 15.7/16.1, 30.8/31.9, and 15.0/15.8, respectively. At week-24, least squares (LS) mean change from baseline in ISS7 (range: 0-21) was -10.2/-6.0 (dupilumab/placebo, respectively) (LS mean difference -4.2; P=0.0005) and for UAS7 (range: 0-42) was -20.5/-12.0 (difference -8.5; P=0.0003).

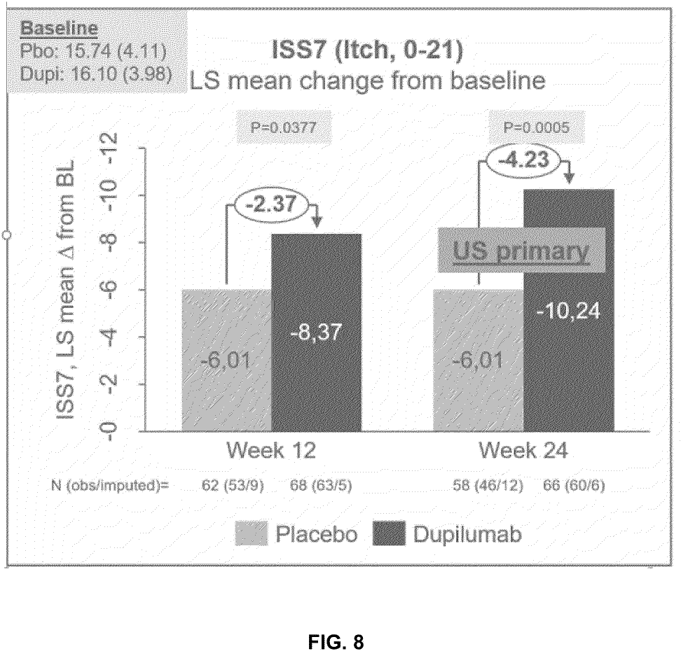

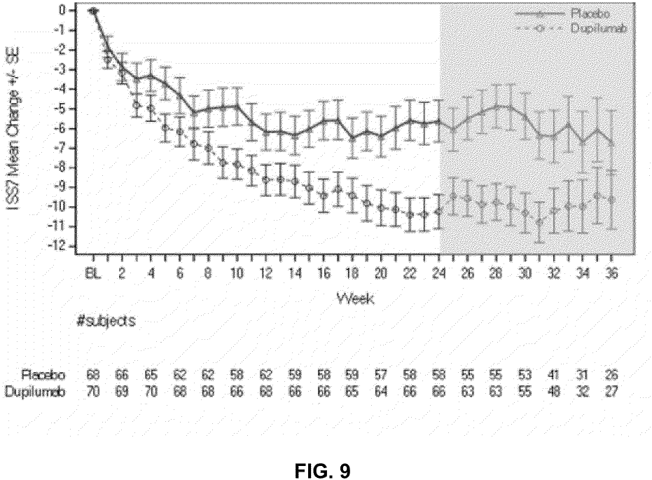

The ISS7 primary endpoint was met with clinical and statistical significance. The results for ISS7 in least square mean (LS mean) change from baseline is graphically depicted in FIG. 8. At 12 weeks, the ISS7 (range 0-21) (LS mean from baseline) in the dupilumab group was -8.37 and -6.01 in the placebo group (difference of -2.37, p=0.0377.) At 24 weeks, the ISS7 (LS mean from baseline) in the dupilumab group was -10.24 and -6.01 in the placebo group (difference of -4.23, p=0.0005.) FIG. 9 depicts a plot of mean change in ISS7 over time in both the placebo and dupilumab treatment groups from baseline to week 36. The range of the ISS7 score is 0-21 with a minimally important difference (MID) range of 4.5-5.

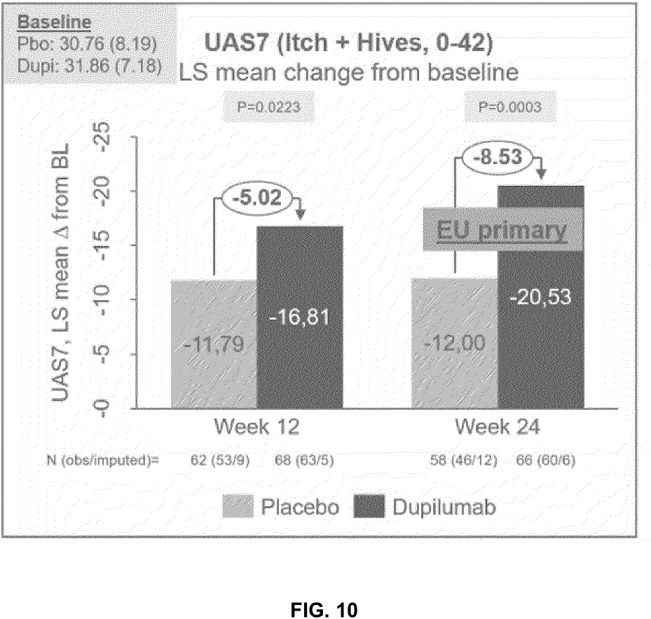

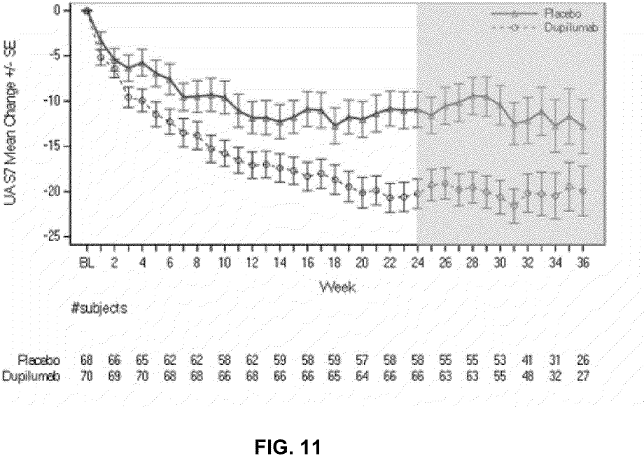

The UAS7 primary endpoint was met with clinical and statistical significance. The results for UAS7 in least square mean (LS mean) change from baseline is graphically depicted in FIG. 10. At 12 weeks, the UAS7 (range: 0-42) (LS mean from baseline) in the dupilumab group was -16.81 and -11.79 in the placebo group (difference of -5.02, p=0.0223.) At 24 weeks, the UAS7 (LS mean from baseline) in the dupilumab group was -20.53 and -12 in the placebo group (difference of -8.53, p=0.0003.) FIG. 11 depicts a plot of mean change in UAS7 over time in both the placebo and dupilumab treatment groups from baseline to week 36. The range of the UAS7 score is 0-42 with a minimally important difference (MID) range of 9.5-10.5.

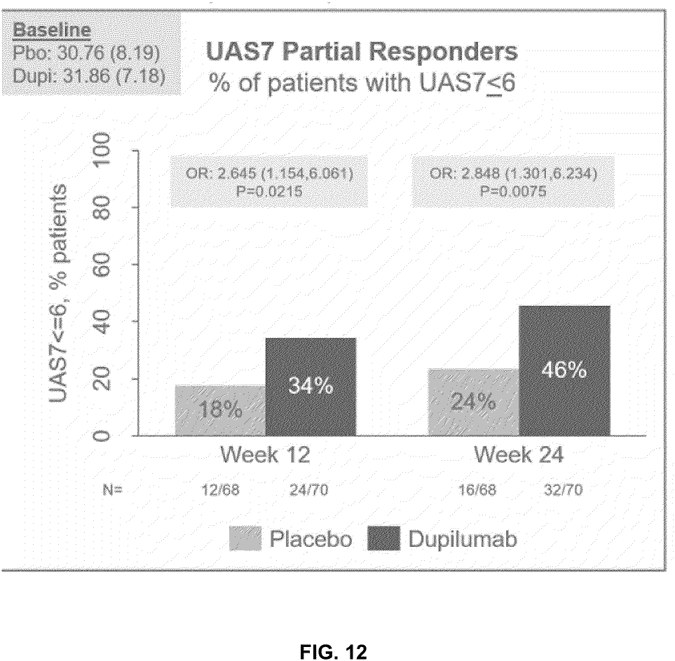

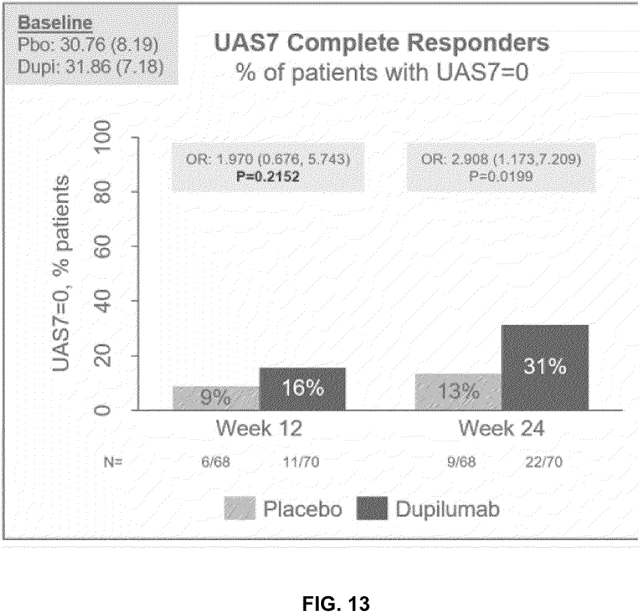

The percentage of UAS7 partial and complete responders in the dupilumab treatment versus placebo group at 24 weeks was statistically significant. FIG. 12 graphically depicts the percentage of UAS7 partial responders (patients with UAS7 equal to or less than 6) in both the placebo and dupilumab treatment groups at 12 and 24 weeks. At 12 weeks, 18% of the placebo group were partial responders and 34% of the dupilumab treatment group were partial responders (p=0.0215.) At 24 weeks, 24% of the placebo group were partial responders and 46% of the dupilumab treatment group were partial responders (p=0.0075.) FIG. 13 graphically depicts the percentage of UAS7 complete responders (patients with UAS7 equal to zero) in both the placebo and dupilumab treatment groups at 12 and 24 weeks. At 12 weeks, 9% of the placebo group were partial responders and 16% of the dupilumab treatment group were complete responders (p=0.2152.) At 24 weeks, 13% of the placebo group were partial responders and 31% of the dupilumab treatment group were partial responders (p=0.0199.)

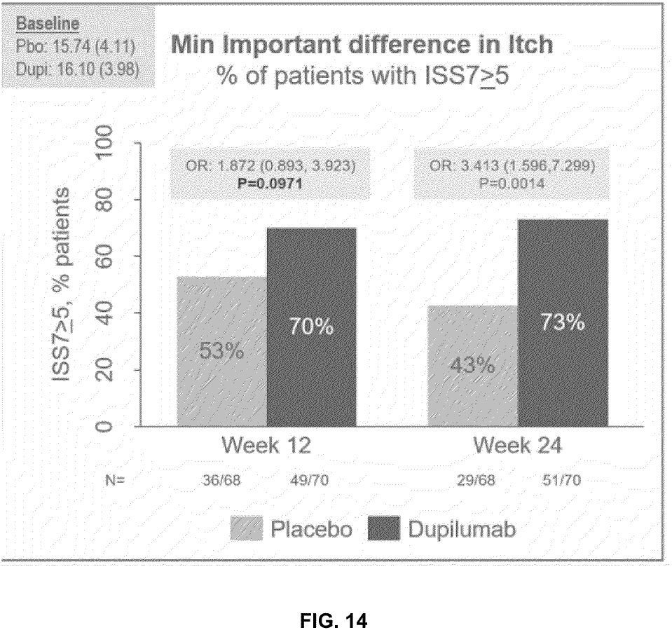

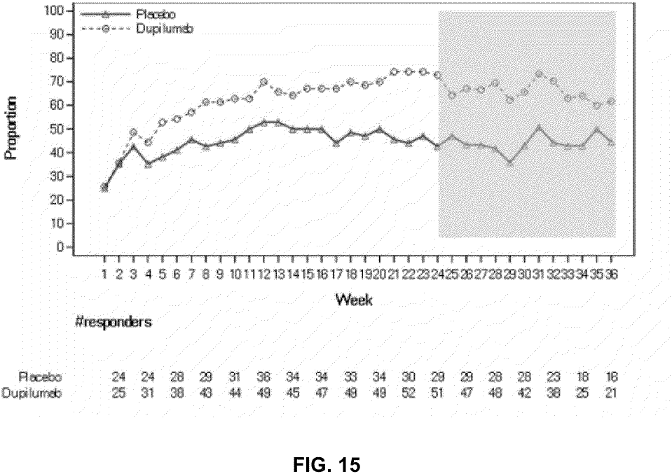

The percentage of patients with an ISS7 minimum important difference (MID) (patients with a decrease in ISS7 of 5 or greater) was statistically significant at week 24 for the dupilumab treatment versus placebo group. FIG. 14 graphically depicts the percentage of patients that reached the ISS7 MID in both the placebo and dupilumab treatment groups at 12 and 24 weeks. At week 12, 53% of placebo and 70% of dupilumab treated patients reached ISS7 MID (p=0.0971.) At week 24, 43% of placebo and 73% of dupilumab treated patients reached ISS7 MID (p=0.0014.) FIG. 15 depicts a plot of the proportion of patients with an ISS7 reduction from baseline of 5 points or more over time in both the placebo and dupilumab treatment groups up to week 36.

At 24 weeks, the LS mean change in HSS7 (range:0-21) from baseline was -10.3 in the dupilumab group and -5.9 in the placebo group (difference of -4.4, P=0.0003).

Dupilumab significantly reduced itch, as measured by ISS7, at week 24 regardless of whether baseline serum total IgE was < 100 IU/mL or ≥ 100 IU/mL (median serum total IgE of the overall population at baseline was 101.0 IU/mL): ISS7 LS mean difference vs placebo (95% confidence interval) -4.24 (-7.86, -0.62) and -4.63 (-8.22, -1.04), respectively. Additionally, dupilumab significantly reduced urticaria activity, as measured by UAS7, at week 24 regardless of baseline serum total IgE level: UAS7 LS mean difference vs placebo (95% confidence interval) -8.17 (-15.04, -1.29) and -10.63 (-17.72, -3.54) for IgE < 100 IU/mL or ≥ 100 IU/mL, respectively; HSS7, -4.2 (-7.60, -0.70) / -6.1 (-9.95, -2.33); UAS7, -8.2 (-15.04, -1.29) / -10.6 (-17.72, -3.54). Occurrence of treatment-emergent adverse events (TEAEs) for dupilumab/placebo were 35 (50.0%) / 40 (58.8%); injection-site reactions, 8 (11.4%) / 9 (13.2%); conjunctivitis, 0/1 (1.5%); serious TEAEs, 2 (2.9%) / 5 (7.4%).

Dupilumab demonstrated clinically meaningful and statistically significant improvements in patients with H1 antihistamine-resistant CSU regardless of baseline IgE level, and was well-tolerated.

### Safety Results Study A

Overall treatment-emergent adverse events (TEAEs) were comparable between dupilumab and placebo: 35 (50.0%)/40 (58.8%); occurrence of injection site reactions was 8 (11.4%)/9 (13.2%), conjunctivitis 0/1 (1.5%), and serious TEAEs 2 (2.9%)/5 (7.4%). TEAE, severe TEAE, TEAE leading to treatment discontinuation, and SAE were observed more frequently on placebo vs. dupilumab (1 death in placebo (suicide), 1 conjunctivitis in placebo, more incidence of skin disorders (18 vs. 9) in the placebo group than dupilumab treatment (including angioedema 5 in placebo vs. 1 in dupilumab treatment.) There was a comparable incidence of site reactions (9 in placebo vs. 8 in dupilumab treatment.) Two AESIs were observed in the dupilumab treatment group (pregnancy and hypersensitivity). The summary of the TEAEs is shown in Table 9 below. A further breakdown of the TEAEs is provided in Table 10. Overall treatment-emergent adverse events reported in at least 5% of patients in any treatment group were comparable for placebo and dupilumab.

TABLE 9

| n (%) | Placebo (N=68) | Dupilumab (N=70) |
|---|---|---|
| Participants with any TEAE | 40 (58.8) | 35 (50.0) |
| Participants with any severe TEAE | 4 (5.9) | 2 (2.9) |
| Participants with any treatment emergent SAE | 5 (7.4) | 2 (2.9) |
| Participants with any TEAE leading to death | 1 (1.5) | 0 |
| Participants with any TEAE leading to permanent study intervention discrimination | 4 (5.9) | 2 (2.9) |
| Participants with any treatment emergent AES1 | 0 | 2 (2.9) |
| Participants with any treatment emergent other selected AE | 10 (14.7) | 9 (12.9) |
| Participants with any TEAE related to IMP | 16 (23.5) | 10 (14.3) |

TABLE 10

| n (%) | Placebo (n = 68) | Dupilumab (n = 70) |
|---|---|---|
| Any event Skin and subcutaneous tissue disorders CSU Angioedema General disorders and administration site conditions injection-site reaction^{a} injection-site erythema | 40 (58.8) 18 (26.5) 6 (8.8) 5 (7.4) 10 (14.7) 2 (2.9) 4 (5.9) | 35 (50.0) 9 (12.9) 3 (4.3) 1 (1.4) 9 (12.9) 4 (5.7) 3 (4.3) |

^{a} Injection-site reactions by MedDRA High Level Term, n (%): placebo 9 (13.2); dupilumab 8 (11.4). Includes injection-site erythema, injection-site induration, injection-site pain, injection-site pruritus, and injection-site reactions.
Conjunctivitis, n (%): placebo 1 (1.5); dupilumab 0. Conjunctivitis cluster includes conjunctivitis, allergic conjunctivitis, bacterial conjunctivitis, viral conjunctivitis, giant papillary conjunctivitis, eye irritation, and eye inflammation. MedDRA, Medical Dictionary for Regulatory Activities.

Overall, dupilumab was well tolerated and demonstrated an acceptable safety profile in CSU. The safety profile was consistent with the known safety profile of dupilumab observed in the approved populations and indications. No new safety signal was reported in CSU patients. There were no suspected unexpected serious adverse events reported (SUSARs), no serious cardiovascular events, and no malignancy.

Patient Disposition: Patient Disposition for Study A is shown in Table 11 below. Three times more drop-outs were observed in the placebo (27%) versus dupilumab treatment (9%) groups. The treatment policy was to use available data after drop-out. MI stands for missing data. Worst-observation carried forward (WOCF) was used for missing data if the reason was lack of efficacy per investigator.

TABLE 11

Patient Disposition

| n (%) | Placebo (N-68) | Dupilumab (N-70) | All (N-138) |
|---|---|---|---|
| Expired and not Randomized | 0 | 0 | 0 |
| Randomized and not expired | 0 | 0 | 0 |
| Randomized and exposed | 68 (100) | 70 (100) | 138 (100) |
| Completed the study intervention period Did not complete the study intervention period | 50 (71.3) 18 (26.5) | 64 (91.4) 6 (8.6) | 114 (82.6) 24 (17.4) |
| Reason for permanent study intervention withdrawal Adverse evens | 4 (2.9) | 2 (0 | 6 (4.3) |
| Related to COVID-19 | 0 | 2.9) | 0 |
| Not related to COVID-19 | 4 (2.9) | 2 (2.9) | 6 (4.3) |
| Lack of efficiency | 4 (2.9) | 1 (1.4) | 5 (3.6) |
| Peer compliance to protocol | 0 | 0 | 0 |
| Withdrawal by subject | 8 (11.8) | 2 (2.9) | 10 (7.2) |
| Other Related to COVID-19 | 2 (2.9) 0 | 1 (1.4) 0 | 3 (2.2) 0 |
| Not related to COVID-19 | 2 (2.9) | 1 (1.4) | 3 (2.2) |
| Reason for study intervention withdrawal by subject Adverse event | 1 (1.2) | 0 | 1(0.7) |
| Related to COVID-19 | 0 | 0 | 0 |
| Not related to COVID-19 | 1 (1.5) | 0 | 1 (0.7) |
| Study procedure | 0 | 0 | 0 |
| Lack of efficiency | 3 (4.4) | 2 (2.9) | 2 (3.6) |
| Other | 4 (5.9) | 0 | 4 (2.9) |
| Related to COVID-19 | 0 | 00 0 | 0 |
| Not related to COVID-19 | 4 (5.9) | 0 | 4 (2.9) |
| Completed the study period | 29 (42.8) | 32 (42.7) | 61 (44.2) |
| Did not complete the study period | 12 (17.6) | 4 (8.6) | 18 (13.02) |
| Ongoing in study follow-up | 27 (99.3) | 32 (42.7) | 32 (42.5) |
| Reason for study discontinuation | | | |
| Adverse event | 3 (4.4) | 1 (1.4) | 4 (2.9) |
| Peer compliance in protocol | 0 | 0 | 0 |
| Withdrawal by subject | 7 (10.3) | 3 (4.3) | 10 (3.2) |
| Site terminated by expenses | 0 | 0 | 0 |
| Study terminated by expenses | 0 | 0 | 0 |
| Other | 2 (2.9) | 2 (2.9) | 4 (2.9) |
| Related to COVID-19 | | 0 | 0 |
| Not related to COVID-19 | 0 2 (2.9) | 2 (2.9) | 4 (2.9) |
| Status at test experiment | | | |
| Alive Dead | 39 (37.4) 1 (1.2) | 38 (34.3) 0 | 77 (55.8) 1 (0.3) |

Demographics: Patient demographics from Study A are shown in Table 12, below. A total of 6 pediatric patients were included, 2 children 6-11 years old in the dupilumab group and 2 adolescents from 12-17 years old in each of the placebo and dupilumab groups. A total of 12 elderly patients, older than 65, were included in Study A. More female patients (66%) were included.

TABLE 12

Demographics

| | Placebo (N-68) | Dupilumab (N-70) | All (N-138) |
|---|---|---|---|
| Age (years) Number | 68 | 70 | 138 |
| Mass(SD) Median Q1 : Q3 Min : Max | 41.9 (14.8) 41.0 28.5; 52.5 12; 77 | 40.7 (16.2) 41.5 27.0; 52.0 8; 7.5 | 41.3 (15.5) 41.0 28.0; 52.0 8; 77 |
| Age group (years) [n (%)] Number | 68 | 70 | 138 |
| 6-11 12-17 | 0 2 (2.9) | 2 (2.9) 2 (2.9) | 2 (1.4) 4 (2.9) |
| 18-39 40-64 | 30 (44.1) 32 (47.1) | 28 (40.0) 30 (42.9) | 58 (42.0) 62 (44.9) 10 (7.2) |
| 65-74 >75 | 3 (4.4) 1(1.5) | 7 (10.0) 1 (1.4) | 2 (1.004) |
| Region [n (%)] Number | 68 | 70 | 138 |
| Asia LatinAmerican East Europe Western Countries | 16 (23.5) 10 (14.7) 12 (17.6) 30 (44.1) | 17 (24.3) 9 (12.9) 11 (15.007) 33 (47.1) | 33 (23.9) 19 (13.008) 23 (16.7) 63 (45.7) |
| Tertiary [n (%)] Numbers | 68 | 70 | 138 |
| North America European Union & United Kingdom Rest of World | 27 (39.7) 4 (5.9) 37 (54.4) | 29 (41.4) 4(5.7) 37 (52.9) | 56 (40.6) 8 (5.8) 74 (53.6) |
| Sex [n(%)] Number | 68 | 70 | 138 |
| Male Female | 18 (26.5) 50 (73.5) | 29 (41.4) 41 (58.6) | 47 (34.1) 91 (65.9) |
| Race [n(%)] Number | 68 | 70 | 138 |
| White Black or African American Asian Japanese | 48 (70.6) 2 (2.9) 16 (23.5) 6 (8.8) | 47 (67.1) 1 (1.4) 19 (27.001) 6 (8.6) | 95 (68.8) 3 (2.2) 35 (23.4) 12 (8.7) |
| Native Hawaiian or Other Pacific Islander | 0 | 0 | 0 |
| American Indian or Alaska Native Multiple Not reported Unknown | 0 1 (1.5) 1 (1.5) 0 | 1 (1.4) 1 (1.4) 1 (1.4) 0 | 1 (0.7) 2 (1.4) 2 (1.4) 0 |
| Ethnicity [n (%)] Number Hispanic or Latino Not Hispanic or Latino | 65 12 (17.6) 54 (82.4) | 70 13 (18.6) 57 (81.4) | 138 25 (18.1) 113 (81.9) |
| Weight (kg) Number Mass (SD) Medium Q1 : Q3 Min; Max | 67 75.79 (18.36) 72.00 64.50; 87.00 43.0; 119.9 | 70 77.15 (21.49) 74.00 60.00; 92.90 32.5; 135.9 | 137 76.48 (19.96) 73.00 63.00; 91.99 32.5; 135.9 |
| Weight group (kg) [n (%)] Number <50 50 to <100 100 | 67 4 (6.0) 53 (82.1) 8 (11.9) | 70 3 (4.3) 58 (82.9) 9 (12.9) | 137 7(5.1) 113 (82.5) 17 (12.4) |
| BMI (kg/m*) Number Mass (SD) Medium Q1 : Q3 Min : Max | 67 27.99 (6.19) 27.15 23.15 : 30.76 176 : 44.8 | 70 27.45 (6.77) 26.19 22.90 : 31.77 17.3 : 53.1 | 137 27.07 (6.47) 26.77 22.22 : 30.82 173 : 53.1 |
| BMI group (kg/m*)[n (%)] Number <30 >30 | 67 47 (70.1) 30 (29.9) | 70 51 (72.9) 19 (27.1) | 137 98 (78.5) 39 (28.5) |

Disease Characteristics: The disease characteristics of the patients in Study A are summarized in Table 13, below. Baseline ISS7 was about 16 in both dupilumab treatment and placebo groups. Baseline UAS7 was about 31 in both dupilumab treatment and placebo groups. Baseline HSS7 was about 15 in both dupilumab treatment and placebo groups. About 45% of the patients in Study A had angioedema. Baseline total IgE was about 50% below and 50% above 100 in both dupilumab treatment and placebo groups. Baseline H1-antihistamines was about 50% standard dose and 50% a higher than standard dose in both dupilumab treatment and placebo groups.

TABLE 13

Disease Characteristics

| | Placebo (N-68) | Dupilamax (N-70) | All (N-138) |
|---|---|---|---|
| Age & ease of CSU (year) Number | 68 | 70 | 138 |
| Mean (SD) Median Q1:Q3 Min:Max | 36.7(16.0) 36.3 24.0,30.0 3:77 | 38.3 (16.8) 20.065.0 20.066.0 6.33 | 38.1 (16.2) 37.0 24.0 : 50.0 5 : 77 |
| Time score first diagnosis of CSU (years) Number | 68 | 70 | 138 |
| Mass (SD) Median Q1 : Q3 Min : Max | 585 (7.70) 282 1.00,3.33 06 ; 33.0 | 3.77 (0.32) 2.04 0.92 ; 5.67 05 ; 60.0 | 5.71 (8.53) 2.04 0.92 : 6.00 0.5 : 60.0 |
| Baseline 1597 score Number | 68 | 70 | 138 |
| Mean (SD) | 134(4.1) | 16.1 (4.0) | 15.9 (4.0) |
| Median Q1:Q3 Min:Max | 13.5 13.0; 20.0 8;21 | 16.0 14.0, 20.0 8;21 | 16.0 13.0 : 20.0 8 : 21 |
| Baseline 1557 score [n(%)] Number | 68 | 70 | 138 |
| <33 ≥13 | 16 (23.5) 52 (76.5) | 12 (17.1) 38 (82.9) | 28 (20.3) 110 (79.7) |
| Baseline UAS7 score Number | 68 | 70 | 138 |
| Mean (SD) | 30.8 (8.2) | .31.9 (7.2) | 33.3 (7.7) |
| Median | 31.9 | 32.5 | 32.0 |
| Q1 : Q3 Min:Max | 24.0 ; 37.5 16;42 | 28.0 ; 32.0 16.42 | 260 : 37.0 16 : 42 |
| Baseline UAS7 more [n(%)] Number | 68 | 70 | 138 |
| <22 ≥28 | 24 (33.8) 44 (64.7) | 17 (24.3) 53 (25.7) | 41 (29.7) 97 (70.3) |
| Baseline HSS7 score Number | 68 | 70 | 138 |
| Mean (SD) (SD) | 15.0 (4.8) | 15.8 (3.8) | 15.4 (4.3) |
| Median Q1 : Q3 Min : Max | 15.5 10.5 : 19.5 2 : 21 | 16.0 14.0 : 19.0 8 : 21 | 16.0 12.0 : 19.0 2 : 21 |
| Presence of angioedema baseline (b (%)] | 68 | 70 | 138 |
| Number Yes No | 34 (50.0) 34 (50.000) | 28 (40.0) 42 (60.0) | 63(44.9) 76(55.1) |
| Baseline AAS? score for participants with angioedema | | | |
| Number Mean (SD) Median Q1 : Q3 Min : Max | 34 15.3 (21.4) 28.0 14.0 : 33.0 4 : 93 | 28 32.1 (23.2) 33.5 25 : 43.0 4 : 92 | 62 33.9(25.4) 31.5 13.0 :48.0 4: 95 |
| Baseline UCT score | | | |
| Number Mean (SD) Median Q1 : Q3 Min : Max | 68 3.7 (2.3) 3.5 2.0 : 3.0 0 : 0 | 68 1.8(2.3) 4.0 2.0 : 5.0 0: 10 | 137 3.1(2.3) 4.0 2.0 : 5.0 0 : 10 |
| Baseline DLQ3/CDLQ1 score | | | |
| Number Mean (SD) Medim Q1 : Q3 Min : Max | 68 15.3 (6.7) 16.0 10.0 : 20.0 2 : 28 | 70 13.6 (6.0) 13.0 100 : 18.0 2 : 27 | 138 14.4(6.4) 14.0 10.0 :20.0 2:28 |
| Baseline PGIS score | | | |
| Number Mean (SD) Median Q1 : Q3 Min : Max | 68 3.5 (0.6) 3.0 3.0 : 4.0 2 : 4 | 70 3.4(0.6) 3.0 3.0 : 4.0 1 : 4 | 138 3.4(0.6) 3.0 3.0 : 4.0 1 : 4 |
| Baseline Total 1 gE (IU/mL) | | | |
| Number Mean (SD) Median Q1 : Q3 Min : Mass | 65 442.327(1851.821) 96.560 22.900 : 221.000 1.00 : 11827.00 | 66 636.777(2886.350) 109.350 49.200 : 283.000 5.21 : 23388.00 | 131 540.294(2421.420) 101.000 38.000: 274.000 1.00: 23388.00 |
| Baseline Total 1 gE [a (%)] | | | |
| Number <100 ≥100 | 65 34 (52.1) 11 (47.1) | 66 31 (47.0) 35(38.0) | 131 61(49.6) 66 (50.4) |
| Baseline H1-antihistamines | | | |
| Number standard dose 2-3-fold standard dose 4-fold standard dose | 68 41(60.3) 17(25.0) 19(14.7) | 70 32(45.7) 26(37.1) 13(12.1) | 138 2y 71(52.9) 43(31.2) 22(15.9) |

Pre-Defined Medical/Surgical History: The medical/surgical history of participants in Study A is shown in Table 14, below. The medical/surgery history of these patients is in line with the published literature on CSU and co-morbid atopic disease. Active atopic dermatitis was an exclusion criteria.

TABLE 14

Pre-Defined Medical/Surgical History

| n(%) | Placebo (N-68) | Dupilumab (N-70) | All (N-138) |
|---|---|---|---|
| Any medical or surgical history* | 54 (79.4) | 48 (68.6) | 102 (73.9) |
| Angioedma ongoing | 40 (58.8) 38 (55.9) | 32 (45.7) 29 (41.4) | 72 (52.2) 07 (48.6) |
| Allergic rhinitis ongoing | 22 (32.4) 22 (32.4) | 18 (25.7) 16 (22.9) | 40 (29.0) 38 (27.5) |
| Asthma ongoing | 12 (17.6) 11 (16.2) | 9 (12.9) 8 (11.4) | 21 (15.2) 19 (13.8) |
| Allergic conjunctivitis ongoing | 10 (14.7) 10 (14.7) | 4 (5.7) 4 (5.7) | 14 (10.1) 14 (10.1) |
| Food allergy ongoing | 5 (7.4) 5 (7.4) | 6 (8.6) 6 (8.6) | 11 (8.0) 11 (8.0) |
| Atopic dermatitis ongoing | 5 (7.4) 0 | 3 (4.3) 0 | 8 (5.8) 0 |
| Chronic rhinosinusitis ongoing | 4 (3.9) 3 (4.004) | 2 (2.9) 2 (2.9) | 6 (4.3) 5 (3.6) |
| Nasal polyps ongoing | 2 (2.9) 0 | 3 (4.3) 2 (2.9) | 5 (3.6) 2 (1.4) |
| Eosinophilic esophagitis ongoing | 0 0 | 0 0 | 0 0 |

Rescue Medication: A summary of rescue medications as recorded by the investigator during the 24-week treatment period is presented in Table 15, below.

TABLE 15

Summary of Rescue Medications

| | Placebo (N-69) | Dupilumab (N-70) |
|---|---|---|
| Any rescue medication Increased dose of H1-antihistamaines BILASTINE CETIRIZINE HYDROCHLORIDE EBASTINE RUPATADINE FUMARATE Short course OCS DEXAMETHASONE METHYLPREDNISOLONE PREDNISOLONE PREDNISONE | 6 (8.8) 4 (5.9) 2 (2.9) 1 (1.5) 1 (1.5) 2 (2.9) 5 (7.4) 1 (1.5) 1 (1.5) 3 (4.4) 1 (1.5) | 4 (5.7) 1 (1.4) 0 0 0 1 (1.4) 3 (4.3) 0 0 0 3 (4.3) |

OCS: oralcorticostroids

Study A Pediatric Patients Data Summary: A total of 6 pediatric patients were randomized. 4 adolescents aged 12-17 years old were included. 2 adolescent patients received dupilumab and both patients showed a reduction in CSU signs and symptoms (one adolescent patient had a complete response.) For the 2 adolescent patients in the placebo group, 1 stopped treatment at the patient’s decision for lack of efficacy and 1 patient reported a reduction in signs and symptoms of CSU. Two children aged 6-11 years both of which were on dupilumab treatment, prematurely stopped treatment as the patient’s decision for lack of efficacy (one stopped at week 4, the other at week 10). Overall dupilumab showed good safety in pediatric CSU patients. No TEAEs were reported in any pediatric patients.

### Study A Conclusions

Overall, dupilumab demonstrated clinically meaningful and statistically significant efficacy in patients with CSU who remain symptomatic despite the use of H1 antihistamine treatment, and was well tolerated.

Furthermore, these results were unexpected due to the fact that the role of IL-4 and IL-13 in the pathogenesis of CSU was considered uncertain. (See Gimenez-Arnau AM, et al. The Pathogenesis of Chronic Spontaneous Urticaria: The Role of Infiltrating Cells J Allergy Clin Immunol Pract 2021 Jun;9(6):2195-2208.) In particular, “the pathogenesis of chronic spontaneous urticaria (CSU) typically focus[ed] on mechanisms by which cutaneous mast cells (MCs) may be activated to initiate the process.” (Id. at page 2195.) Additionally, “the basophil is thought to have an important role in the pathogenesis of CSU given its similarities to the MC as a major source of histamine and expression of the high-affinity receptor for IgE.” (Id. at page 2199.) Further, it was known that “circulating IL-4, as well as IL-4 produced by PBMCs, does not seem elevated in the majority of patients with CSU” and “no correlation with disease activity or ASST [autologous serum skin test] could be made.” (Id. citing Degirmenci et al. Analysis of the Association of Chronic Spontaneous Urticaria with Interlekin-4, -10, Transforming Growth Factor-β1, Interferon-y, Interleukin-17A and -23 by Autologous Serum Skin Test. Postepy Dermatol Alergol. 2017 Feb;34(1):70-76 and Confino-Cohen R, et al. Low Stimulated IL-4 Secretion in PBMC from Patients with Chronic Idiopathic Urticaria. Cytokine. 2004 Jul 21-Aug 7;27(2-3):74-80.) It was also known that “IL-13 serum levels in patients with CSU do not correlate with disease activity.” (Giménez-Arnau AM, et al. The Pathogenesis of Chronic Spontaneous Urticaria: The Role of Infiltrating Cells J Allergy Clin Immunol Pract 2021 Jun;9(6):2195-2208 citing Chen et al. Different Expression Patterns of Plasma Th1-, Th2-, Th17- and Th22-related Cytokines Correlate with Serum Autoreactivity and Allergen Sensitivity in Chronic Spontaneous Urticaria. JEur Acad Dermatol Venereol. 2018 Mar;32(3):441-448.) Thus, the results provided in this study which demonstrate that an antibody that inhibits IL-4 and IL-13 was efficacious in treating CSU were unexpected.

The phase 3 CSU clinical trial met its primary endpoints and all key secondary endpoints. At 24 weeks, dupilumab nearly doubled reduction in itch and urticaria activity score (itch and hives) regardless of baseline IgE level. Adding dupilumab to standard-of-care antihistamines significantly reduced itch and hives for biologic-naive patients, compared to those treated with antihistamines alone (placebo) in Study A.

In the trial (n=138), adding dupilumab to standard-of-care antihistamines nearly doubled the reduction in itch and urticaria activity (itch and hives) compared to standard-of-care alone at 24 weeks, with sustained effect observed beyond active treatment period. The trial results showed a 63% reduction in itch severity with dupilumab vs 35% with standard-of-care (antihistamines) as measured by a 22-point itch severity scale (10.24-point improvement with dupilumab vs 6.01-point improvement with standard-of-care, p=0.0005), the primary endpoint in the US (secondary endpoint in the EU).

The trial results showed a 65% reduction in urticaria activity (itch and hive) severity with dupilumab vs 37% with standard-of-care, as measured by a 43-point urticaria activity scale (20.53-point improvement with dupilumab vs 12.00-point improvement with standard-of-care, p=0.0003), the primary endpoint in EU (secondary endpoint in the US). Additionally, among dupilumab-treated patients 73% experienced a clinically meaningful difference on itch and 31% experienced complete disease control, compared to 43% and 13% in standard of care patients, respectively (p<0.02).

Other endpoints, including responder analysis for UAS7 and ISS7 were met at 24 weeks. ISS7 and UAS7 at week 12 were also statistically significant.

Notably, treatment with dupilumab did not plateau by week 24, was sustained during the 12 week follow up period, and was consistent regardless of baseline IgE levels.

The trial demonstrated safety results similar to the known safety profile of dupilumab in its approved dermatological indications. For the 24-week treatment period, overall rates of treatment emergent adverse events were generally similar between the dupilumab and placebo groups (50% for dupilumab and 59% for standard of care). The most common adverse events were injection site reactions (11% dupilumab, 13% standard of care). Conjunctivitis was reported in 0 dupilumab patients and 1 standard of care patient. Overall TEAE, severe TEAE, TEAE leading to treatment discontinuation, and SAE were observed more frequently in the placebo versus dupilumab treatment group.

### Study B Conclusions

Study B of the CSU clinical program evaluated dupilumab in patients with chronic spontaneous urticaria (CSU), who were refractory to omalizumab, and was recently stopped due to futility based on a pre-specified interim analysis, although numeric improvements were observed across key endpoints. The safety data were generally consistent with Study A and the known safety profile of dupilumab in its approved dermatological indications.

### Example 2. A Multi-Center, Single-Arm Study to Investigate the Pharmacokinetics and Safety of Dupilumab in Male and Female Participants ≥2 Years to <12 Years of Age With Uncontrolled Chronic Spontaneous Urticaria (CSU)

### Study Rationale

Chronic urticaria is defined by the appearance of itchy wheals (hives) with or without angioedema for more than 6 weeks. Chronic spontaneous urticaria (CSU) is urticaria for which no triggering factor is identified. Blockade of IL-4/IL-13 by dupilumab represents a potential novel therapeutic approach for CSU patients.

While antihistamines are the mainstay of therapy, up to 50% of patients may remain uncontrolled with antihistamines alone. Targeting IgE with omalizumab has been successful in treating CSU patients but not all patients are responsive to this therapy, nor is it approved for patients under 12 years of age. Treatment of pediatric patients with CSU remains challenging; the pathophysiology of these conditions is thought to be the same across all age groups, thus antihistamines are first-line therapy. However, there remains a significant unmet need for novel therapies for these indications, particularly in the pediatric population.

### Study Design

This is a Phase 3, multicenter, single-arm, 24-week treatment study assessing the PK and safety of dupilumab in participants ≥2 years to <12 years of age with uncontrolled chronic spontaneous CSU.

The primary objective of this study is to characterize the PK profile, and the secondary objective is to assess the safety profile of dupilumab in children aged ≥2 years to <12 years with uncontrolled CSU. This study will additionally collect clinical information regarding the response to treatment in this age group, however all efficacy analyses will be descriptive.

The study consists of 3 periods:
Screening period (2 to 4 weeks)
Study intervention period (24 weeks)
Follow up period (12 weeks)
The study duration will be 38 to 40 weeks (including screening and follow-up)
The number of study visits will be 8.

### Screening Period

Prior to screening, participants must be receiving treatment for CSU with a non-sedating H1-antihistamine.

The duration of the screening period will be 2 to 4 weeks.

### Treatment Period

After successful completion of the screening period, participants will begin the treatment period. All participants will be administered dupilumab subcutaneously (SC) every 4 weeks (Q4W) or every 2 weeks (Q2W) with or without an initial loading dose based on weight and age. Post-Treatment Period

All participants will complete a 12-week post-treatment follow-up period without study intervention after completing their treatment period. Investigational Medicinal Product
Dupilumab (SAR231893; DUPIXENT).

Formulation Injection solution.

Route of Administration subcutaneous (SC).

### Objectives

### Primary Objectives

To characterize the serum concentration of dupilumab over time.

### Secondary Objectives

To assess the safety of dupilumab.
To assess the immunogenicity of dupilumab.
To evaluate the improvement in health-related QoL in participants receiving dupilumab with CSU who remain symptomatic despite the use of H1-antihistamine or appropriate preventative measures.
To assess the impact of dupilumab on urticaria activity, itch and hives severity scores in participants with CSU who remain symptomatic despite the use of H1-antihistamine.

Endpoints
Concentration of dupilumab in serum over time including C_{trough} at week 12 and week 24.
Safety and tolerability assessments: Incidence of TEAEs or SAEs.
Incidence of ADA to dupilumab over time.
Change from baseline in C-DLQI in children from 4 years to less than 12 years of age at week 24.
Change from baseline in IDQOL in children from 2 years to less than 4 years of age at week 24.
Change from baseline in the modified UAS7 at week 24.
Change from baseline in the modified ISS7 at week 24.
Change from baseline in the modified HSS7 at week 24.

### Inclusion Criteria

Participants are eligible to be included in the study only if all of the following criteria apply:
Age
   Participant must be ≥ 2 years to < 12 years of age, at the time of signing the informed consent.
Type of participant and disease characteristics
   Participants who have a documented diagnosis of CSU >6 months prior to screening visit.
Participants with CSU
   Participants with CSU (characterized by recurrent itchy wheals with or without angioedema for >6 consecutive weeks) who remain symptomatic at the time of screening despite regular H1-antihistamine treatment.
Weight
   Body weight within ≥5 kg to <60 kg.
Compliance
   Participant/parent(s)/caregiver(s)/participant’s legally authorized representative, as appropriate, willing and able to comply with study visits and related procedures.

### Exclusion Criteria

Participants are excluded from the study if any of the following criteria apply:

### Medical Conditions

Underlying etiology for chronic urticarias other than CSU.

Presence of skin morbidities other than CSU that may interfere with the assessment of the study outcomes.

Participants with a concurrent diagnosis of both CSU and CICU.

Participants with active AD.

Severe concomitant illness(es) that would adversely affect the patient’s participation in the study.

Participants with active tuberculosis (TB) or non-tuberculous mycobacterial infection, or a history of incompletely treated TB.

Diagnosed with, suspected of, or at high risk of endoparasitic infection.

Active chronic or acute infection requiring treatment with systemic antibiotics, antivirals, or antifungals within 2 weeks before the screening visit (V1) or during the screening period.

Known or suspected immunodeficiency.

Active malignancy or history of malignancy within 5 years before the baseline visit.

History of systemic hypersensitivity or anaphylaxis to dupilumab including any excipient.

Participation in prior dupilumab clinical study or have been treated with commercially available dupilumab.

1. A method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and
wherein the subject was previously ineffectively treated with H1 antihistamine therapy and anti-IgE antibody therapy.

2. The method of claim 1, wherein:
(a) the subject remains symptomatic despite the use of H1 antihistamine; or
(b) an H1 antihistamine is administered in combination with the antibody or an antigen-binding fragment thereof, optionally wherein the H1 antihistamine is selected from the group consisting of cetirizine, levocetirizine, fexofenadine, loratadine, desloratadine, bilastine, and rupatadine; or
(c) the subject is intolerant to omalizumab or remains symptomatic despite the use of omalizumab.

3-5. (canceled)

6. The method of claim 1, wherein:
(a) the subject is an adult, wherein the antibody or antigen-binding fragment thereof is administered to the subject as an initial dose of about 600 mg followed by one or more secondary doses of about 300 mg, and wherein each secondary dose is administered every 2 weeks; or
(b) the subject is 12 years old to less than 18 years old and has a body weight greater than or equal to 30 kg and less than 60 kg, wherein the antibody or antigen-binding fragment thereof is administered to the subject as an initial dose of about 400 mg followed by one or more secondary doses of about 200 mg, and wherein each secondary dose is administered every 2 weeks; or
(c) the subject is 12 years old to less than 18 years old and has a body weight of at least 60 kg, wherein the antibody or antigen-binding fragment thereof is administered to the subject as an initial dose of about 600 mg followed by one or more secondary doses of about 300 mg, and wherein each secondary dose is administered every 2 weeks; or
(d) the subject is 6 years old to less than 12 years old and has a body weight of at least 30 kg, wherein the antibody or antigen-binding fragment thereof is administered to the subject as an initial dose of about 400 mg followed by one or more secondary doses of about 200 mg, and wherein each secondary dose is administered every 2 weeks; or
(e) the subject is 6 years old to less than 12 years old and has a body weight of at least 30 kg and less than 60 kg, wherein the antibody or antigen-binding fragment thereof is administered to the subject as an initial dose of about 400 mg followed by one or more secondary doses of about 200 mg, and wherein each secondary dose is administered every 2 weeks; or
(f) the subject is 6 years old to less than 12 years old and has a body weight of less than 30 kg and at least 15 kg, wherein the antibody or antigen-binding fragment thereof is administered to the subject as an initial dose of about 600 mg followed by one or more secondary doses of about 300 mg, and wherein each secondary dose is administered every 4 weeks; or
(g) the subject is 6 years old to less than 12 years old and has a body weight of less than 30 kg and at least 15 kg, wherein the antibody or antigen-binding fragment thereof is administered to the subject as an initial dose of about 300 mg followed by one or more secondary doses of about 300 mg, and wherein each secondary dose is administered every 4 weeks; or
(h) the subject is at least 6 years old and less than 12 years old and has a body weight of less than 15 kg and at least 5 kg, wherein the antibody or antigen-binding fragment thereof is administered to the subject as an initial dose of about 200 mg followed by one or more secondary doses of about 200 mg, and wherein each secondary dose is administered every 4 weeks; or
(i) the subject is at least 2 years old and less than 6 years old and has a body weight of less than 30 kg and at least 15 kg, wherein the antibody or antigen-binding fragment thereof is administered to the subject as an initial dose of about 300 mg followed by one or more secondary doses of about 300 mg, and wherein each secondary dose is administered every 4 weeks; or
(j) the subject is at least 2 years old and less than 6 years old and has a body weight of less than 15 kg and at least 5 kg, wherein the antibody or antigen-binding fragment thereof is administered to the subject as an initial dose of about 200 mg followed by one or more secondary doses of about 200 mg, and wherein each secondary dose is administered every 4 weeks; or
(k) the subject is at least 2 years old and less than 6 years old and has a body weight of between at least 30 kg and less than 60 kg, wherein the antibody or antigen-binding fragment thereof is administered to the subject as an initial dose of about 400 mg followed by one or more secondary doses of about 200 mg, and wherein each secondary dose is administered every two weeks.

7-27. (canceled)

28. A method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is 12 years old to less than 18 years old,
wherein the antibody or antigen-binding fragment thereof is administered to the subject as an initial dose followed by one or more secondary doses, and
wherein the antibody or antigen-binding fragment thereof is administered to the subject in a weight-dependent dosage; or
a method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is 6 years old to less than 12 years old,
wherein the antibody or antigen-binding fragment thereof is administered to the subject as an initial dose followed by one or more secondary doses, and
wherein the antibody or antigen-binding fragment thereof is administered to the subject in a weight-dependent dosage; or
a method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is 2 years old to less than 6 years old,
wherein the antibody or antigen-binding fragment thereof is administered to the subject as an initial dose followed by one or more secondary doses, and
wherein the antibody or antigen-binding fragment thereof is administered to the subject in a weight-dependent dosage.

29. The method of claim 28, wherein:
(a) the subject is 12 years old to less than 18 years old and subject has a body weight greater than or equal to 30 kg and less than 60 kg,wherein the initial dose is about 400 mg and each secondary dose is about 200 mg, and wherein each secondary dose is administered every 2 weeks; or
(b) the subject is 12 years old to less than 18 years old and has a body weight of at least 60 kg, wherein the antibody or antigen-binding fragment thereof is administered to the subject as an initial dose of about 600 mg followed by one or more secondary doses of about 300 mg, and wherein each secondary dose is administered every 2 weeks; or
(c) the subject is 6 years old to less than 12 years old and has a body weight of at least 30 kg, wherein the antibody or antigen-binding fragment thereof is administered to the subject as an initial dose of about 400 mg followed by one or more secondary doses of about 200 mg, and wherein each secondary dose is administered every 2 weeks; or
(d) the subject is 6 years old to less than 12 years old and has a body weight of at least 30 kg and less than 60 kg, wherein the antibody or antigen-binding fragment thereof is administered to the subject as an initial dose of about 400 mg followed by one or more secondary doses of about 200 mg, and wherein each secondary dose is administered every 2 weeks; or
(e) the subject is 6 years old to less than 12 years old and has a body weight of less than 30 kg and at least 15 kg, wherein the antibody or antigen-binding fragment thereof is administered to the subject as an initial dose of about 600 mg followed by one or more secondary doses of about 300 mg, and wherein each secondary dose is administered every 4 weeks; or
(f) the subject is 6 years old to less than 12 years old and has a body weight of less than 30 kg and at least 15 kg, wherein the antibody or antigen-binding fragment thereof is administered to the subject as an initial dose of about 300 mg followed by one or more secondary doses of about 300 mg, and wherein each secondary dose is administered every 4 weeks; or
(g) the subject is at least 6 years old and less than 12 years old and has a body weight of less than 15 kg and at least 5 kg, wherein the antibody or antigen-binding fragment thereof is administered to the subject as an initial dose of about 200 mg followed by one or more secondary doses of about 200 mg, and wherein each secondary dose is administered every 4 weeks; or
(h) the subject is at least 2 years old and less than 6 years old and has a body weight of less than 30 kg and at least 15 kg, wherein the antibody or antigen-binding fragment thereof is administered to the subject as an initial dose of about 300 mg followed by one or more secondary doses of about 300 mg, and wherein each secondary dose is administered every 4 weeks; or
(i) the subject is at least 2 years old and less than 6 years old and has a body weight of less than 15 kg and at least 5 kg, wherein the antibody or antigen-binding fragment thereof is administered to the subject as an initial dose of about 200 mg followed by one or more secondary doses of about 200 mg, and wherein each secondary dose is administered every 4 weeks; or
(j) the subject is at least 2 years old and less than 6 years old and has a body weight of between at least 30 kg and less than 60 kg, wherein the antibody or antigen-binding fragment thereof is administered to the subject as an initial dose of about 400 mg followed by one or more secondary doses of about 200 mg, and wherein each secondary dose is administered every two weeks.

30-36. (canceled)

37. The method of claim 28, wherein:
(a) an H1 antihistamine is administered in combination with the antibody or an antigen-binding fragment thereof, or
(b) the subject remains symptomatic despite the use of H1 antihistamine; or
(c) an H1 antihistamine selected from the group consisting of cetirizine, levocetirizine, fexofenadine, loratadine, desloratadine, bilastine, and rupatadine is administered in combination with the antibody or an antigen-binding fragment thereof.

38. A method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is the subject is 12 years old to less than 18 years old,
wherein the antibody or antigen-binding fragment thereof is administered to the subject as an initial dose followed by one or more secondary doses,
wherein the antibody or antigen-binding fragment thereof is administered to the subject in a weight dependent dosage, and
wherein the subject was previously ineffectively treated with antihistamine therapy or anti-IgE antibody therapy, or
a method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is the subject is 6 years old to less than 12 years old,
wherein the antibody or antigen-binding fragment thereof is administered to the subject as an initial dose followed by one or more secondary doses,
wherein the antibody or antigen-binding fragment thereof is administered to the subject in a weight dependent dosage, and
wherein the subject was previously ineffectively treated with antihistamine therapy or anti-IgE antibody therapy; or
a method for treating a subject having chronic spontaneous urticaria (CSU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is 2 years old to less than 6 years old,
wherein the antibody or antigen-binding fragment thereof is administered to the subject as an initial dose followed by one or more secondary doses,
wherein the antibody or antigen-binding fragment thereof is administered to the subject in a weight dependent dosage, and
wherein the subject was previously ineffectively treated with antihistamine therapy or anti-IgE antibody therapy.

39. The method of claim 38, wherein the subject is 12 years old to less than 18 years old, and wherein:
the subject has a body weight greater than or equal to 30 kg and less than 60 kg, the initial dose is about 400 mg and each secondary dose is about 200 mg, and each secondary dose is administered every 2 weeks; or
the subject has a body weight of at least 60 kg, the initial dose is about 600 mg and each secondary dose is about 300 mg, and each secondary dose is administered every 2 weeks.

40-42. (canceled)

43. The method of claim 38, wherein the subject is 6 years old to less than 12 years old, and wherein:
the subject has a body weight of at least 30 kg, the initial dose is about 400 mg and each secondary dose is about 200 mg, and each secondary dose is administered every 2 weeks; or
the subject has a body weight of less than 30 kg and at least 15 kg, the initial dose is about 600 mg and each secondary dose is about 300 mg, and each secondary dose is administered every 4 weeks; or
the subject has a body weight of less than 60 kg, or
the subject has body weight of between at least 5 kg and less than 15 kg, the initial dose is about 200 mg and each secondary dose is about 200 mg, and each secondary dose is administered every four weeks.

44-46. (canceled)

47. The method of claim 38, wherein:
(a) the subject remains symptomatic despite the use of H1 antihistamine; or
(b) an H1 antihistamine is administered in combination with the antibody or an antigen-binding fragment thereof; or
(c) an H1 antihistamine selected from the group consisting of cetirizine, levocetirizine, fexofenadine, loratadine, desloratadine, bilastine, and rupatadine is administered in combination with the antibody or an antigen-binding fragment thereof.

48-58. (canceled)

59. The method of claim 38, wherein the subject is intolerant to omalizumab or remains symptomatic despite the use of omalizumab.

60-72. (canceled)

73. The method of claim 1, wherein the treatment results in an improvement in one or more patient-reported outcomes (PROs) selected from the group consisting of itch severity score (ISS), hive severity score (HSS), urticaria activity score (UAS), angioedema activity score (AAS), urticaria control test (UCT), dermatology life quality index (DLQI), children’s dermatology quality life quality Index (CDLQI), chronic urticaria quality of life questionnaire (CU-Q2oL), patient global impression of change (PGIC), patient global impression of severity (PGIS), Euroqol-5 dimensions (EQ-5D), and Euroqol-5 dimensions Youth version (EQ-5D Y).

74. The method of claim 73, wherein:
(a) the PRO is itch severity score (ISS) and the subject has a decrease in itch severity score over 7 days (ISS7), optionally wherein the decrease in ISS7 is at least 5; or (b) the PRO is hive severity score (HSS) and the subject has a decrease in hive severity score over 7 days (HSS7), optionally wherein the decrease in HSS7 is at least 10 at 24 weeks of treatment; or
(c) the PRO is urticaria activity score (UAS) and the subject has a decrease in urticaria activity score over 7 days (UAS7), optionally wherein the decrease in UAS7 is at least 10; or
(d) wherein the PRO is urticaria activity score (UAS) and the UAS7 of the subject is 0; or
(e) the PRO is urticaria activity score (UAS) and the UAS of the subject is 6 or less; or
(f) the PRO is angioedema activity score over 7 days (AAS7) and the subject has a decrease in AAS score; or
(g) the PRO is the urticaria control test (UCT) and the subject has an increase in UCT score, optionally wherein the UCT of the subject is 12 or greater; or
(h) the PRO is dermatology life quality index (DLQI) and the subject has a decrease in DLQI score; or
(i) the PRO is children’s dermatology life quality index (CDLQI) and the subject has a decrease in CDLQI score; or
(i) the PRO is chronic urticaria quality of life questionnaire (CU-Q2oL) and the subject has a decrease in CU-Q2oL score; or
(k) the PRO is patient global impression of change (PGIC) and the subject has a decrease in PGIC score; or
(1) the PRO is patient global impression of severity (PGIS) and the subject has a decrease in PGIS score; or
(m) the PRO is Euroqol-5 dimensions (EQ-5D) or Euroqol-5 dimensions Youth version (EQ-5D Y) and the subject has an increase in EQ visual analogue scale (EQ VAS) score.

75-90. (canceled)

91. The method of claim 73, wherein the improvement in the PRO occurs with 12 weeks of treatment with the antibody or antigen-binding fragment thereof or wherein the improvement in the PRO occurs with 24 weeks of treatment with the antibody or antigen-binding fragment thereof.

92. (canceled)

93. The method of claim 1, wherein:
(a) prior to treatment with the antibody or antigen-binding fragment thereof, the subject has an UAS7 score of 16 or more; or
(b) prior to treatment with the antibody or antigen-binding fragment thereof, the subject has an ISS7 score of 8 or more; or
(c) prior to treatment with the antibody or antigen-binding fragment thereof, the subject has angioedema; or
(d) the treatment with the antibody or antigen-binding fragment thereof results in an increase in the number of itch-free days that the subject experiences; or
(e) the treatment with the antibody or antigen-binding fragment thereof results in an increase in the number of hive-free days that the subject experiences.

94-97. (canceled)

98. The method of claim 1, wherein the treatment with the antibody or antigen-binding fragment thereof results in a decrease in the need for treatment of the subject with oral corticosteroids, and wherein the dosage of oral corticosteroids required is decreased or the number of days wherein oral corticosteroid treatment is required are decreased.

99. (canceled)

100. (canceled)

101. The method of claim 1, wherein the treatment with the antibody or antigen-binding fragment thereof results in a decrease in the need for treatment of the subject with antihistamine rescue medication, and wherein the dosage of antihistamine rescue medication required is decreased or the number of days wherein antihistamine rescue medication is required is decreased.

102. (canceled)

103. (canceled)

104. The method of claim 1, wherein:
(a) the antibody or antigen-binding fragment thereof comprises a heavy chain variable region (HCVR) sequence of SEQ ID NO: 1 and a light chain variable region (LCVR) sequence of SEQ ID NO: 2; or
(b) the antibody is dupilumab; or
(c) the antibody or antigen-binding fragment thereof is administered using an autoinjector, a needle and syringe, or a pen.

105. (canceled)

106. (canceled)

107. The method of claim 104,wherein the antibody or antigen-binding fragment thereof is administered using a prefilled device and/or the antibody or antigen-binding fragment thereof is administered subcutaneously.

108-117. (canceled)

118. The method of claim 1, wherein the subject does not have active atopic dermatitis and/or wherein the subject does not have chronic inducible cold urticaria (CICU).

119-131. (canceled)
